(19) United States
(12) Patent Application Publication
(10) Pub. No.: US 2012/0064592 A1
(43) Pub. Date: Mar. 15, 2012

(54) BIOCATALYSTS SYNTHESIZING DEREGULATED CELLULASES

(75) Inventors: Patrick O'Mullan, South Grafton, MA (US); Jase Patel, Brighton, MA (US); Matthias Schmalisch, Marlborough, MA (US); Branden Wolner, Auburn, MA (US); Philippa Reeder, Arlington, MA (US)

(73) Assignee: Qteros, Inc., Marlborough, MA (US)

(21) Appl. No.: 13/270,166

(22) Filed: Oct. 10, 2011

Related U.S. Application Data
(60) Provisional application No. 61/436,575, filed on Jan. 26, 2011.

(51) Int. Cl. C12P 7/10; C12P 7/02; C12M 1/00; C12N 1/20

(52) U.S. Cl. 435/165; 435/252.7; 435/155; 435/289.1

(57) ABSTRACT

Provided are isolated novel Clostridium phytofermentans biocatalysts with deregulated cellulase activity that produce high yields of products. Further provided are methods of using the biocatalysts to degrade organic material and for use in industrial processes.

### CROSS-REFERENCE

This application claims the benefit of U.S. Provisional Application No. 61/436,575, filed Jan. 26, 2011, which application is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Oct. 7, 2011, is named 37836740.txt and is 128,772 bytes in size.

### BACKGROUND

Increasing cost of petroleum-based transportation fuels, dwindling petroleum reserves and concerns over the environmental impact of petroleum-fuel combustion are driving a strong demand for viable alternatives to replace petroleum-based fuels. In particular, recent years have highlighted the promise of producing biofuels through bio-conversion of a variety of pretreated biomass material, such as lignocellulosic material, starch, or agriculture waste/byproducts, in combination with enzymes and yeast/bacterial systems. A particular challenge is developing technology with the potential to economically convert polysaccharide containing materials such as woody or nonwoody plant material, algae, and nonvascular plants as well as waste materials and side products from the processing of plant or algal matter into high value transportation fuels and other energy forms or chemical feedstocks. Various examples of these polysaccharide containing materials include cellulosic, lignocellulosic, and hemicellulosic material; pectin containing material; starch; wood; corn stover; bagasse; switchgrass; distillers' grains, paper; and paper pulp sludge, and the like.

Many options for generating effective and sustainable biofuels and other biochemicals have been studied. Bioenergy sources can include alcohols, diesel, gases, bioelectricity (microbial fuel cells) and specialty chemicals. Ethanol fermentation from biomass including cellulosic, lignocellulosic, pectin, polyglucose and/or polyfructose containing biomass can provide much needed solutions for the world energy problem. A first and economically important step is the conversion of cellulosic and hemicellulosic polymers to oligomers and monomers (saccharification). Incomplete conversions affect the product yields. Slow fermentations run the risk of contamination from unwanted species of bacteria and fungi and result in unwanted products. Methods to increase rates of hydrolysis without the addition of expensive exogenous enzymes would clearly reduce the cost of the products of fermentation through biocatalysts.

Many species of yeast, fungi and bacteria have been reported to be able to convert cellulosic biomass of its monomeric sugars to ethanol. However, products of hydrolysis can adversely affect the rate of cellulase synthesis in these organisms during fermentation. See, e.g., Nataf, Y., Bahari, L., Kahel-Raifer, H., Borovok, I., Lamed, R., Bayer, E. A., Sonenshein, A. L., Shoham, Y. (2010). Clostridium thermocellum cellulosomal genes are regulated by extracytoplasmic polysaccharides via alternative sigma factors. Proc. Natl. Acad. Sci. USA 107: 18646-18651; Abdou, L., Boileau, C., de Philip, P., Pages, S., Fierobe, H.-P., Tardif, C. (2008). Transcriptional Regulation of the Clostridium cellulolyticum cip-cel Operon: a Complex Mechanism Involving a Catabolite-Responsive Element. (2008). J. Bacteria 190: 1499-1506. Cellulase activity in most organisms is regulated and synthesis of cellulase enzymes is subject to feedback inhibition, especially from glucose and other sugar monomers. Control of such feedback inhibition or suppression mechanism present in the organism, through regulation of synthesis or another molecular modification can boost ethanol and chemical productivity.

### SUMMARY

Disclosed herein are isolated microorganisms that produce a fermentation end-product from a biomass, the microorganisms comprising a genetic modification that enables the microorganisms to synthesize more cellulases in the presence of an inhibitor molecule than a microorganism of the same species without the genetic modification. In one embodiment, In one embodiment, the inhibitor molecule is glucose or a glucose analog. In one embodiment, the genetic modification comprises a mutation in one or more genes, wherein at least one of the genes encodes a propionyl-CoA carboxylase, a two component AraC family transcriptional regulator, or a ROK family glucokinase, a homolog of Cphy_{—}3487, a dihydrolipoamide dehydrogenase, a binding-protein-dependent transport systems inner membrane component, an ABC transporter related protein, a homolog of Cphy_{—}0056, a TetR family transcriptional regulator, an AraC-like protein, a glycosyl transferase 36, a diaminopimelate epimerase, an oxidoreductase domain-containing protein, a homolog of Cphy_{—}2965, a desulfoferrodoxin ferrous iron-binding region, a homolog of Cphy_{—}1063, an AraC family transcriptional regulator, a phage tape measure protein, a D-isomer specific 2-hydroxyacid dehydrogenase NAD-binding protein, or an HD superfamily phosphohydrolase-like protein. In one embodiment, the fermentation end-product is an alcohol. In one embodiment, the alcohol is ethanol. In one embodiment, the biomass comprises hemicellulosic or lignocellulosic material. In one embodiment, the microorganism can hydrolyze and ferment hemicellulosic or lignocellulosic material. In one embodiment, the microorganism is a Clostridium species.

Also disclosed herein are methods of producing a fermentation-end product comprising: providing a biomass in a media; contacting the biomass with an isolated microorganism comprising a genetic modification that enables the microorganism to synthesize more cellulases in the presence of an inhibitor molecule than a microorganism of the same species without the genetic modification; and, allowing sufficient time for the microorganism to produce the fermentation end-product from the biomass. In one embodiment, the inhibitor molecule is glucose or a glucose analog. In one embodiment, the fermentation end-product is an alcohol. In one embodiment, the alcohol is ethanol. In one embodiment, the biomass comprises hemicellulosic or lignocellulosic material. In one embodiment, the microorganism can hydrolyze and ferment hemicellulosic or lignocellulosic material. In one embodiment, the microorganism is a Clostridium species.

Also disclosed herein are plants for producing a fermentation end product comprising: a fermenter, wherein the fermenter is configured to house a biomass in a medium; and, an isolated microorganism comprising a genetic modification that enables the microorganism to synthesize more cellulases in the presence of an inhibitor molecule than a microorganism of the same species without the genetic modification. In one embodiment, the inhibitor molecule is glucose or a glucose analog. In one embodiment, the fermentation end-product is an alcohol. In one embodiment, the alcohol is ethanol. In one embodiment, the biomass comprises hemicellulosic or lignocellulosic material and wherein the microorganism can hydrolyze and ferment the hemicellulosic or lignocellulosic material. In one embodiment, the microorganism is a Clostridium species.

Disclosed here are isolated microorganisms that produce a fermentation end-product from a biomass, the microorganisms comprising a genetic modification that enables the microorganism to synthesize more cellulases in the presence of an inhibitor molecule than a microorganism of the same species without the genetic modification. In one embodiment, the increased cellulase synthesis is characterized by a larger clearing zone when the microorganism is grown on a phosphoric acid-swollen cellulose (PASC) agar plate in comparison to a non-genetically modified microorganism of the same species. In one embodiment, the clearing zone is measured after staining with Congo-Red. In one embodiment, the clearing zone is measured according to a radius, diameter, area, or volume of the clearing zone. In one embodiment, the increased cellulase synthesis is characterized in a fluorescent cellulase activity assay. In one embodiment, the fluorescent cellulase activity assay comprises 4-methylumbilliferone (4Mu) substrates. In one embodiment, the 4Mu substrates comprise glucopyranoside, cellobioside, or a combination thereof. In one embodiment, the fluorescent cellulase activity assay shows about a 5 to about a 10 fold increase in cellulase activity for the microorganism in comparison to the microorganism of the same species without the genetic modification. In one embodiment, the increased cellulase synthesis is characterized an increased level of cellulase coding mRNA. In one embodiment, the increased cellulase synthesis is characterized by an increased level of cellulase protein. In one embodiment, the microorganism produces more of the fermentation end-product with the genetic modification than without the genetic modification. In one embodiment, the inhibitor molecule is glucose or a glucose analog. In one embodiment, the genetic modification comprises a mutation in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, or a combination thereof. In one embodiment, the genetic modification comprises a mutation in one or more genes. In one embodiment, the mutation reduces an activity, stability, or expression level of a protein encoded by at least one of the genes. In one embodiment, the mutation increases an activity, stability, or expression level of a protein encoded by at least one of the genes. In one embodiment, at least one of the genes encodes a propionyl-CoA carboxylase, a two component AraC family transcriptional regulator, or a ROK family glucokinase. In one embodiment, at least one of the genes encodes a propionyl-CoA carboxylase, a two component AraC family transcriptional regulator, or a ROK family glucokinase, a homolog of Cphy_{—}3487, a dihydrolipoamide dehydrogenase, a binding-protein-dependent transport systems inner membrane component, an ABC transporter related protein, a homolog of Cphy_{—}0056, a TetR family transcriptional regulator, an AraC-like protein, a glycosyl transferase 36, a diaminopimelate epimerase, an oxidoreductase domain-containing protein, a homolog of Cphy_{—}2965, a desulfoferrodoxin ferrous iron-binding region, a homolog of Cphy_{—}1063, an AraC family transcriptional regulator, a phage tape measure protein, a D-isomer specific 2-hydroxyacid dehydrogenase NAD-binding protein, or an HD superfamily phosphohydrolase-like protein. In one embodiment, at least one of the genes is a homolog of a gene from Table 4. In one embodiment, at least one of the genes is from Table 4. In one embodiment, at least one of the genes is a ROK family glucokinase. In one embodiment, the mutation is in the carbohydrate biding pocket of the ROK family glucokinase. In one embodiment, at least one of the genes is a two component AraC family transcriptional regulator, wherein the two component AraC family transcriptional regulator controls the expression of one or more genes encoding an ABC transporter protein. In one embodiment, at least one of the genes is a propionyl-CoA carboxylase. In one embodiment, the one or more genes comprises a combination of genes as indicated in Table 4. In one embodiment, the microorganism can hydrolyze and ferment hemicellulosic or lignocellulosic material. In one embodiment, the microorganism is a bacteria, a yeast, or another fungus. In one embodiment, the microorganism is a Clostridium species. In one embodiment, the microorganism is Clostridium phytofermentans, Clostridium sp. Q.D., or a variant thereof. In one embodiment, the microorganism is Clostridium phytofermentans Q.17, Q.18, Q.19, or Q.20. In one embodiment, the microorganism is deposited under NRRL Accession Number B-50447, NRRL Accession Number B-50448, NRRL Accession Number B-50449, or NRRL Accession Number B-50450. In one embodiment, the microorganism produces more of the fermentation end-product from the biomass than a microorganism of the same species without the genetic modification. In one embodiment, the microorganism produces between about 10% and about 100% more of the fermentation end-product from the biomass than a microorganism of the same species without the genetic modification. In one embodiment, the fermentation end-product is an alcohol. In one embodiment, the fermentation end-product is ethanol. In one embodiment, the biomass comprises cellulosic, hemicellulosic, or lignocellulosic material. In one embodiment, the microorganism produces a higher saccharification yield from the biomass than a microorganism of the same species without the genetic modification.

Also disclosed herein are isolated bacterium selected from the group consisting of Clostridium phytofermentans Q.17, Clostridium phytofermentans Q.18, Clostridium phytofermentans Q.19, or Clostridium phytofermentans Q.20.

Also disclosed herein are methods of producing a fermentation-end product comprising: providing a biomass in a media; contacting the biomass with an isolated microorganism comprising a genetic modification that enables the microorganism to synthesize more cellulases in the presence of an inhibitor molecule than a microorganism of the same species without the genetic modification; and, allowing sufficient time for the microorganism to produce the fermentation end-product from the biomass. In one embodiment, the microorganism produces more of the fermentation end-product with the genetic modification than without the genetic modification. In one embodiment, the inhibitor molecule is glucose or a glucose analog. In one embodiment, the fermentation end-product is an alcohol. In one embodiment, the fermentation end-product is ethanol. In one embodiment, the biomass comprises cellulose, hemicellulose, lignocellulose, or a combination thereof. In one embodiment, the biomass comprises hemicellulosic or lignocellulosic material. In one embodiment, the microorganism can hydrolyze and ferment hemicellulosic or lignocellulosic material. In one embodiment, the microorganism is a bacteria, yeast, or another fungus. In one embodiment, the microorganism is a Clostridium species. In one embodiment, the microorganism is Clostridium phytofermentans, Clostridium sp Q.D., or a variant thereof. In one embodiment, the microorganism is Clostridium phytofermentans Q.17, Clostridium phytofermentans Q.18, Clostridium phytofermentans Q.19, or Clostridium phytofermentans Q.20. In one embodiment, the microorganism is deposited under NRRL Accession Number B-50447, NRRL Accession Number B-50448, NRRL Accession Number B-50449, or NRRL Accession Number B-50450. In one embodiment, the genetic modification comprises a mutation in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, or a combination thereof. In one embodiment, the genetic modification comprises a mutation in one or more genes. In one embodiment, the mutation reduces an activity, stability, or expression level of a protein encoded by at least one of the genes. In one embodiment, the mutation increases an activity, stability, or expression level of a protein encoded by at least one of the genes In one embodiment, at least one of the genes encodes a propionyl-CoA carboxylase, a two component AraC family transcriptional regulator, or a ROK family glucokinase. In one embodiment, at least one of the genes encodes a propionyl-CoA carboxylase, a two component AraC family transcriptional regulator, or a ROK family glucokinase, a homolog of Cphy_{—}3487, a dihydrolipoamide dehydrogenase, a binding-protein-dependent transport systems inner membrane component, an ABC transporter related protein, a homolog of Cphy_{—}0056, a TetR family transcriptional regulator, an AraC-like protein, a glycosyl transferase 36, a diaminopimelate epimerase, an oxidoreductase domain-containing protein, a homolog of Cphy2965, a desulfoferrodoxin ferrous iron-binding region, a homolog of Cphy_{—}1063, an AraC family transcriptional regulator, a phage tape measure protein, a D-isomer specific 2-hydroxyacid dehydrogenase NAD-binding protein, or an HD superfamily phosphohydrolase-like protein. In one embodiment, at least one of the genes is a homolog of a gene from Table 4. In one embodiment, at least one of the genes is from Table 4. In one embodiment, at least one of the genes is a ROK family glucokinase. In one embodiment, the mutation is in the carbohydrate biding pocket of the ROK family glucokinase. In one embodiment, at least one of the genes is a two component AraC family transcriptional regulator, wherein the two component AraC family transcriptional regulator controls the expression of one or more genes encoding an ABC transporter protein. In one embodiment, at least one of the genes is a propionyl-CoA carboxylase. In one embodiment, the one or more genes comprises a combination of genes as indicated in Table 4. In one embodiment, the microorganism produces wherein the microorganism produces between about 10% and about 100% more of the fermentation end-product from the biomass than a microorganism of the same species without the genetic modification.

Also disclosed herein are plants for producing a fermentation end product comprising: a fermenter, wherein the fermenter is configured to house a biomass in a medium; and, an isolated microorganism comprising a genetic modification that enables the microorganism to synthesize more cellulases in the presence of an inhibitor molecule than a microorganism of the same species without the genetic modification. In one embodiment, the microorganism produces more of the fermentation end-product with the genetic modification that without the genetic modification. In one embodiment, the inhibitor molecule is glucose or a glucose analog. In one embodiment, the fermentation end-product is an alcohol. In one embodiment, the fermentation end-product is ethanol. In one embodiment, the biomass comprises cellulose, hemicellulose, lignocellulose, or a combination thereof. In one embodiment, the biomass comprises hemicellulosic or lignocellulosic material. In one embodiment, the microorganism can hydrolyze and ferment hemicellulosic or lignocellulosic material. In one embodiment, the microorganism is a bacteria, yeast, or another fungus. In one embodiment, the microorganism is a Clostridium species. In one embodiment, the microorganism is Clostridium phytofermentans, Clostridium sp Q.D., or a variant thereof. In one embodiment, the microorganism is Clostridium phytofermentans Q.17, Clostridium phytofermentans Q.18, Clostridium phytofermentans Q.19, or Clostridium phytofermentans Q.20. In one embodiment, the microorganism is deposited under NRRL Accession Number B-50447, NRRL Accession Number B-50448, NRRL Accession Number B-50449, or NRRL Accession Number B-50450. In one embodiment, the genetic modification comprises a mutation in one or more genes. In one embodiment, the genetic modification comprises a mutation in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, or a combination thereof. In one embodiment, the mutation reduces an activity, stability, or expression level of a protein encoded by at least one of the genes. In one embodiment, the mutation increases an activity, stability, or expression level of a protein encoded by at least one of the genes. In one embodiment, at least one of the genes encodes a propionyl-CoA carboxylase, a two component AraC family transcriptional regulator, or a ROK family glucokinase. In one embodiment, at least one of the genes encodes a propionyl-CoA carboxylase, a two component AraC family transcriptional regulator, or a ROK family glucokinase, a homolog of Cphy_{—}3487, a dihydrolipoamide dehydrogenase, a binding-protein-dependent transport systems inner membrane component, an ABC transporter related protein, a homolog of Cphy_{—}0056, a TetR family transcriptional regulator, an AraC-like protein, a glycosyl transferase 36, a diaminopimelate epimerase, an oxidoreductase domain-containing protein, a homolog of Cphy_{—}2965, a desulfoferrodoxin ferrous iron-binding region, a homolog of Cphy_{—}1063, an AraC family transcriptional regulator, a phage tape measure protein, a D-isomer specific 2-hydroxyacid dehydrogenase NAD-binding protein, or an HD superfamily phosphohydrolase-like protein. In one embodiment, at least one of the genes is a homolog of a gene from Table 4. In one embodiment, at least one of the genes is from Table 4. In one embodiment, at least one of the genes is a ROK family glucokinase. In one embodiment, the mutation is in the carbohydrate biding pocket of the ROK family glucokinase. In one embodiment, at least one of the genes is a two component AraC family transcriptional regulator, wherein the two component AraC family transcriptional regulator controls the expression of one or more genes encoding an ABC transporter protein. In one embodiment, at least one of the genes is a propionyl-CoA carboxylase. In one embodiment, the one or more genes comprises a combination of genes as indicated in Table 4. In one embodiment, the microorganism produces wherein the microorganism produces between about 10% and about 100% more of the fermentation end-product from the biomass than a microorganism of the same species without the genetic modification. In one embodiment, the plant further comprises a hydrolysis tank. In one embodiment, the biomass is pretreated. In one embodiment, the pretreatment comprises acid hydrolysis, base hydrolysis, hot water treatment, enzyme hydrolysis, size reduction, or a combination thereof.

Also disclosed herein are isolated bacteria selected from the group consisting of Q.17, Q.18, Q.19 or Q.20. In one embodiment, the bacterium can hydrolyze polysaccharides in the presence of a repressor molecule. In one embodiment, the polysaccharides are comprised of hexose or pentose sugars. In one embodiment, the bacterium is deposited under NRRL Accession Number B-50447, NRRL Accession Number B-50448, NRRL Accession Number B-50449, or NRRL Accession Number B-50450. In one embodiment, the bacterium can utilize cellulose or xylose as its sole carbon source. In one embodiment, the bacterium produces alcohol dehydrogenase. In one embodiment, the alcohol dehydrogenase reduces acetaldehyde into ethanol. In one embodiment, the ethanol is produced at greater than 90% theoretical yield from biomass. In one embodiment, the polysaccharides comprise a portion of a biomass.

Also disclosed herein are high-yielding mutants of Clostridium phytofermentans that produces ethanol at a rate of over 45 g/l from biomass.

Also disclosed herein are methods of fermenting a biomass material, comprising contacting the biomass material with a culture of Q.17, Q.18, Q.19 or Q.20 biocatalyst. In one embodiment, polysaccharides comprise a portion of a biomass. In one embodiment, the biomass is selected from the group consisting of corn stover, bagasse, lignocellulosic, hemicellulosic material, algae, fruit peels, oat hulls, modified crop plants, pectin containing material, starch, wood, algae, distiller's grains, switchgrass, paper, and paper pulp sludge. In one embodiment, the biomass is pretreated. In one embodiment, the biomass is pretreated to make polysaccharides more available to the biocatalyst. In one embodiment, the biomass is pretreated by pretreatments selected from the group consisting of acid, steam explosion, hot water treatment, alkali, catalase, and a detoxifying or chelating agent. In one embodiment, a fermentation end-product is produced. In one embodiment, the fermentation end product is a chemical. In one embodiment, the fermentation end product is a biofuel. In one embodiment, the fermentation end product is an alcohol. In one embodiment, the fermentation end product is ethanol.

Also disclosed herein are methods of fermenting a biomass material, comprising contacting the biomass material with a culture deposited under NRRL Accession Numbers NRRL B-50436 or NRRL B-50437.

Also disclosed herein are methods of hydrolyzing and fermenting a carbonaceous biomass wherein the biomass is contacted by a C. phytofermentans Q.17, Q.18, Q.19 or Q.20 bacterium for a period long enough to produce ethanol at 70-99.99% theoretical yield from the carbonaceous biomass. In one embodiment, the biomass is contacted by a C. phytofermentans Q.17, Q.18, Q.19 or Q.20 bacterium for a period long enough to produce ethanol at greater than 70% theoretical yield from the carbonaceous biomass. In one embodiment, the biomass is contacted by a C. phytofermentans Q.17, Q.18, Q.19 or Q.20 bacterium for a period long enough to produce ethanol at greater than 80% theoretical yield from the carbonaceous biomass. In one embodiment, the biomass is contacted by a C. phytofermentans Q.17, Q.18, Q.19 or Q.20 bacterium for a period long enough to produce ethanol at greater than 90% theoretical yield from the carbonaceous biomass. In one embodiment, the method further comprises maintaining a temperature at about 30° C. to about 40° C. In one embodiment, the method further comprises maintaining a temperature at about 35° C. to about 39° C. In one embodiment, the contacting is in a medium with a pH from about 5.5 to about 7.5. In one embodiment, the bacterium uses biomass as a major carbon source. In one embodiment, the bacterium is deposited under NRRL Accession Numbers B-50447, B-50448, B-50449, or B-50450. In one embodiment, the bacterium is genetically-modified.

Also disclosed herein are methods for producing a fermentation end-product comprising: (a) culturing a medium comprising a non-recombinant or recombinant Q.17, Q.18, Q.19 or Q.20 biocatalyst for a period of time under conditions suitable for production of a fermentation end-product by the Q.17, Q.18, Q.19 or Q.20 biocatalyst; and (b) harvesting a fermentation end-product from the medium. In one embodiment, the Q.17, Q.18, Q.19 or Q.20 biocatalyst is a mesophile. In one embodiment, the fermentation end-product is a chemical. In one embodiment, the fermentation end-product is a biofuel. In one embodiment, the fermentation end-product is an alcohol. In one embodiment, the fermentation end-product is ethanol. In one embodiment, the medium comprises a cellulosic and/or lignocellulosic material. In one embodiment, the cellulosic or lignocellulosic material is not enzymatically treated with a sufficient quantity of enzymes to convert more than 15% of the cellulosic or lignocellulosic material to simple sugars within 24 hours. In one embodiment, the cellulosic or lignocellulosic material is pretreated by pretreatments selected from the group consisting of acid, steam explosion, hot water treatment, alkali, catalase, and a detoxifying or chelating agent. In one embodiment, a second biocatalyst is added to the medium.

Also disclosed herein are fuel plants comprising a fermenter configured to house a medium and a strain of Q.17, Q.18, Q.19 or Q.20 bacteria, wherein the fermenter comprises a biomass. In one embodiment, the biomass comprises cellulosic or lignocellulosic material. In one embodiment, the biomass is selected from the group consisting of corn stover, bagasse, lignocellulosic, hemicellulosic material, algae, fruit peels, oat hulls, modified crop plants, pectin containing material, starch, wood, algae, distiller's grains, switchgrass, paper, and paper pulp sludge. In one embodiment, the cellulosic or lignocellulosic material is pretreated. In one embodiment, the biomass is pretreated to make polysaccharides more available to the biocatalyst. In one embodiment, the biomass is pretreated by pretreatments selected from the group consisting of acid, steam explosion, hot water treatment, alkali, catalase, and a detoxifying or chelating agent. In one embodiment, the fuel plant is capable of producing a fermentation end-product. In one embodiment, the fermentation end-product is a chemical. In one embodiment, the fermentation end-product is a biofuel. In one embodiment, the fermentation end-product is an alcohol. In one embodiment, the fermentation end-product is ethanol. In one embodiment, the strain is deposited under NRRL Accession Number B-50447, NRRL Accession Number B-50448, NRRL Accession Number B-50449, or NRRL Accession Number B-50450.

Also disclosed herein are isolated mutated or genetically modified microorganisms characterized by deregulated cellulase activity. In one embodiment, the strain is capable of hydrolyzing polysaccharides as a sole carbon source. In one embodiment, the strain is capable of reducing glucose into ethanol at rate of over 45 g/L. In one embodiment, the strain is capable of growth under conditions of elevated ethanol concentration. In one embodiment, the strain is capable of growth under conditions of high sugar concentration. In one embodiment, the strain is capable of growth under conditions of low sugar concentration.

Additional advantages disclosed herein will be set forth in part in the description which follows, and in part will be obvious from the description, or may be learned by practice of the methods described herein. The advantages disclosed herein can be realized and attained by means of the elements and combinations particularly pointed out in the appended claims. It is to be understood that both the foregoing general description and the following detailed description are exemplary and are not restrictive the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features disclosed herein are set forth with particularity in the appended claims. A better understanding of the features and advantages will be obtained by reference to the following detailed description that sets forth illustrative embodiments, in which the principles of the disclosure are utilized, and the accompanying drawings of which:

FIG. 1 shows cellulase activity from 12 cellulase mutant strains grown using glucose as a carbon source compared to C. phytofermentans Q8 (Q.8).

FIG. 2 shows increase in saccharification yields by several strains of C. phytofermentans.

FIG. 3 shows increased ethanol yields of cellulase mutant strains compared to the parent strain.

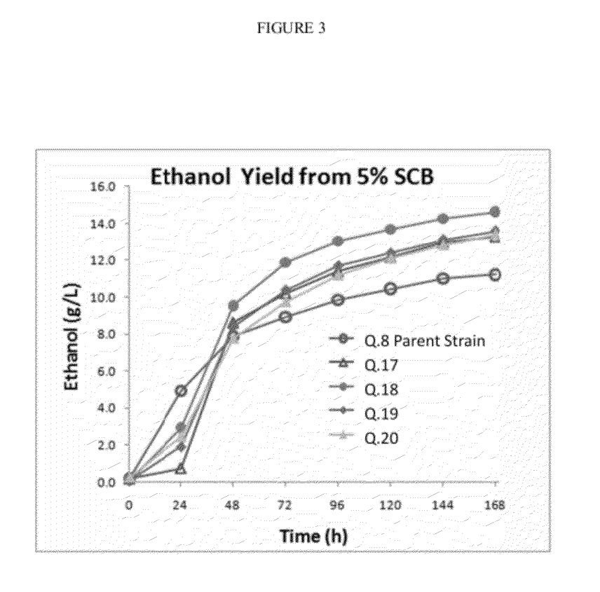

FIG. 4 depicts a method for producing fermentation end products from biomass by first treating biomass with an acid at elevated temperature and pressure in a hydrolysis unit.

FIG. 5 depicts a method for producing fermentation end products from biomass by charging biomass to a fermentation vessel.

FIG. 6 (A-C) discloses pretreatments that produce hexose or pentose saccharides or oligomers that are then unprocessed or processed further and either fermented separately or together.

FIG. 7 illustrates a pathway map for cellulose hydrolysis and fermentation.

FIG. 8 illustrates a plasmid map for pIMP1.

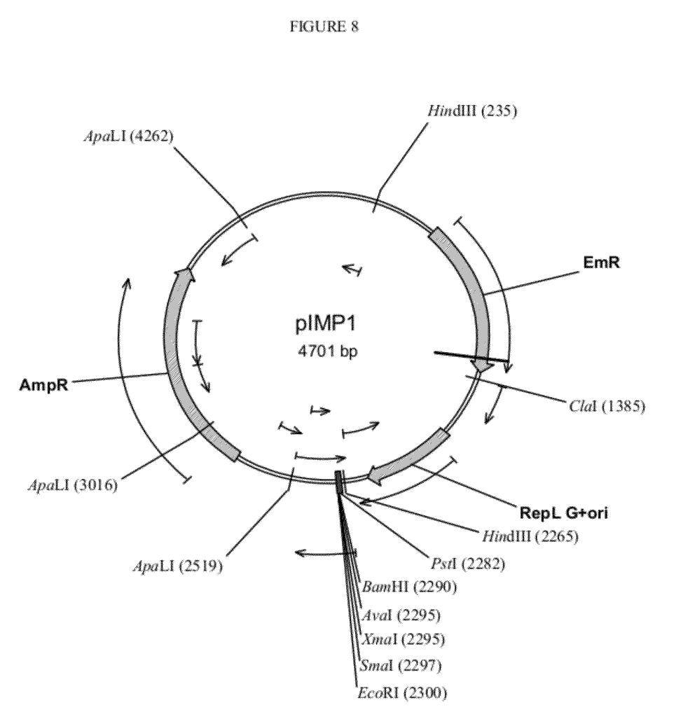

FIG. 9 illustrates a plasmid map for pIMCphy.

FIG. 10 illustrates a plasmid map for pCphyP3510.

FIG. 11 illustrates a plasmid map for pCphyP3510-1163.

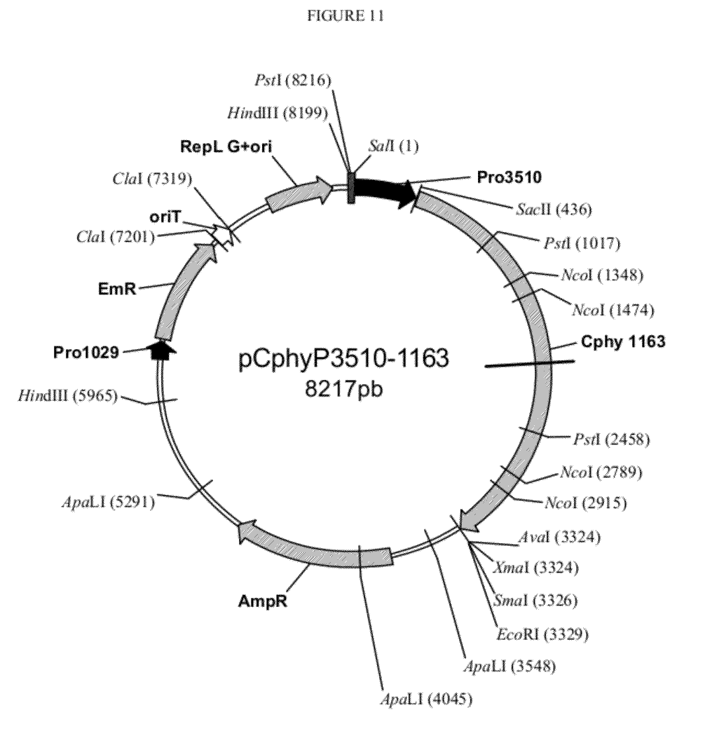

FIG. 12 (A-B) illustrates the protein structure of Streptococcus pneumoniae TIGR4 (PDB:2GUP, in complex with sucrose) which is predicted to have similar structure to Cphy_{—}0329; a mutated residue is pointed to by a white arrow (12A) and a black arrow (12B).

FIG. 13 depicts the plasmid pQInt.

FIG. 14 (A&B) depicts the plasmids pQInt1 and pQInt2.

### DETAILED DESCRIPTION

The present disclosure may be understood more readily by reference to the following detailed description, the Examples included therein and to the Figures and their previous and following description.

Before the present compounds, compositions, articles, devices, and/or methods are disclosed and described, it is to be understood that this disclosure is not limited to specific synthetic methods, specific purified proteins, or to particular nucleic acids, as such may, of course, vary. It is also to be understood that the terminology used herein is for the purpose of describing particular embodiments only and is not intended to be limiting. The following description and examples illustrate some exemplary embodiments of the disclosure in detail. Those of skill in the art will recognize that there are numerous variations and modifications of this disclosure that are encompassed by its scope. Accordingly, the description of a certain exemplary embodiment should not be deemed to limit the scope of the present disclosure.

Deregulated Cellulase Mutants

In microorganisms, synthesis of cellulase enzymes can be subject to feedback inhibition. Control of such feedback inhibition or suppression mechanisms present in the microorganism, through regulation of synthesis or another molecular modification, can boost production of fermentation end-products such as ethanol and chemicals. In one aspect, disclosed herein are microorganism that can produce one or more fermentation end-products from a biomass that comprise a genetic modification to produce higher levels of one or more cellulase enzymes in the presence of an inhibitory molecule, methods of producing a fermentation end-product with the microorganism, and plants for the production of fermentation end products with the microorganism. The cellulase can be any enzyme that hydrolyzes a polysaccharide (e.g., cellulose, hemicellulose, lignocellulose, xylan, glucuranoxylan, arabinoxylan, glucomannan, xyloglucan, callose, chrysolaminarin, mannan, fucoidan, galactomannan, lactose, maltose, sucrose, cellobiose, cellulobiose, etc.). Cellulases can include hemicellulose. Cellulases can include pectinases. The cellulase can be an endocellulase, which can break internal bonds to disrupt the crystalline structure of a matrix polysaccharide (e.g., cellulose, hemicellulose) and can produce individual polysaccharides. The cellulase can be an exocellulase, which can removed two to four subunits form the end of a polysaccharide. The cellulase can be a cellobiose, which can hydrolyze a polysaccharide comprising two to four subunits into monosaccharides. The cellulase can be beta-glucosidase (EC 3.2.1.21); beta-galactosidase (EC 3.2.1.23); beta-mannosidase (EC 3.2.1.25); beta-glucuronidase (EC 3.2.1.31); beta-D-fucosidase (EC 3.2.1.38); phlorizin hydrolase (EC 3.2.1.62); 6-phospho-galactosidase (EC 3.2.1.85); 6-phospho-beta-glucosidase (EC 3.2.1.86); strictosidinebeta-glucosidase (EC 3.2.1.105); lactase (EC 3.2.1.108); amygdalinbeta-glucosidase (EC 1 3.2.1.117); prunasin beta-glucosidase (EC 3.2.1.118); raucaifricine beta-glucosidase (EC 3.2.1.125); thioglucosidase (EC 3.2.1.147); beta-primeverosidase (EC 3.2.1.149); isoflavonod 7-O-beta-apiosyl-glucosidase (EC 3.2.1.161); hydroxyisourate hydrolase (EC_{—}3.-.-.-); beta-glycosidase (EC_{—}3.2.1.-); mannosylglycoprotein 5 endo-beta-mannosidase (EC 3.2.1.152); exo-beta glucosaminidase_(E 3.2.1.-); xylan 1,4-beta-xylosidase (EC 3.2.1.37); beta-N-acetylhexosaminidase (EC 3.2.1.52); glucan 1,3-beta-glucosiclase (EC 3.2.1.58); glucan 1,4-beta-glucosidase (EC 3.2.1.74); exo-1,3-1,4-glucanase (EC 3.2.1.-); alpha-L arabinofuranosidase (EC 3.2.1.55); maltose-6-phosphate glucosidase (EC 3.2.1.122); alpha glucosidase (EC 3.2.1.20); alpha-galactosidase (EC 3.2.1.22); 6-phospho-beta-glucosidase (EC 3.2.1.86); alpha-glucuronidase (EC 3.2.1.139); chitosanase (EC 3.2.1.132); cellulase (EC 3.2.1.4); glucan 1,3-beta-glucosidase (EC 3.2.1.58); licheninase (EC 3.2.1.73); glucan endo-1,6-beta-glucosidase (EC 3.2.1.75); mannan endo-1,4-beta-mannosidase (EC 3.2.1.78); endo-1,4-beta-xylanase (EC 3.2.1.8); cellulose 1,4-beta-cellobiosidase (EC 3.2.1.91); endo-1,6-beta-galactanase (EC 3.2.1.-); beta-1,3-mannanase (EC 3.2.1.-); xyloglucan-specific endo-beta-1,4-glucanase (EC 3.2.1.151); reducing-end-xylose releasing exo-oligoxylanase (EC 3.2.1.156); endoglucanase (EC 3.2.1.4); cellobiohydrolase (EC 3.2.1.91); xylanase (EC 3.2.1.8); endo-1,3-beta-xylanase (EC 3.2.1.32); xyloglucan hydrolase (EC 3.2.1.151); beta-1,3-1,4-glucanase (EC 3.2.1.73); xyloglucan endotransglycosylase (EC 2.4.1.207); apha-amylase (EC 3.2.1.1); pullulanase (EC 3.2.1.41); cyclomaltodextrin glucanotransferase (EC 2.4.1.19); cyclornaltodextrinase (EC 3.2.1.54); trehalose-6-phosphate hydrolase (EC 3.2.1.93); oligo-alpha-glucosiclase (EC 3.2.1.10); maltogenic amylase (EC 3.2.1.133); neopullulanase (EC 3.2.1.135); alpha-glucosidase (EC 3.2.1.20); maltotetraose-forming 3 alpha-amylase (EC 3.2.1.60); isoamylase (EC 3.2.1.68); glucodextranase (EC 12.170); maltohexaose-forming alphaamylase (EC 3.2.1.98); branching enzyme (EC 2.4.1.18); trehalose synthase (EC 5.4.99.16); glucanotransferase (EC 2.4.1.25); maltopentaose-forming-amylase (EC 3.2.1.-); amylosucrase (EC 2.4.1.4): sucrose phosphorylase (EC 2.4.1.7); malto-oligosyltrehalose trehalohydrolase (EC 3.2.1.141); isomaltulose synthase (EC 5.4.99.11); xyloglucan:xyloglucosyltransferase (EC 2.4.1.207); keratan-sulfate endo-1,4-beta-galactosidase (EC 3.2.1.103); Glucan endo-1,3-beta-D-glucosidase (EC 3.2.1.39); endo-1,3(4)-beta-glucanase (EC 3.21.6); Licheninase (EC 3.2.1.73): agarase (EC 3.2.1.81);betacarrageenase (EC 3.2.1.83); xyioglucanase (EC 3.2.1.151); chitinase (EC 3.2.1.14); endo-beta-N-acetylglucosaminidase (EC 3.2.1.96); non-catalytic proteins: xylanase inhibitors; concanavalin B; narbonin; chitinase (EC 3.2.1.14); beta-hexosaminidase (EC 3.2.1.52); lacto-N-biosidase (EC 3.2.1.140); -1,6-N-acetylglucosaminidase) (EC 3.2.1.-); lysozyme (EC 3.2.1.17); beta-mannanase (EC 3.2.1.78);beta-1,3-xylanase (EC 3.2.1.32); polygalacturonase (EC 3.2.1.15); exo-polygalacturonase (EC 3.2.1.67); exo-polygalacturonosidase (EC 3.2.1.82); rhamnogalacturonase (EC 3.2.1.-); endo-xylogalacturonan hydrolase (EC 3.2.1.-); rhamnogalacturonan alpha-L-rhamnopyranohydrolase (EC 3.2.1.40); alpha-L-fucosidase (EC 3.2.1.51); glucosylceramidase (EC 3.2.1.45); beta-1,6-glucanase (EC 3.2.1.75); beta-xylosidase (EC 3.2.1.37); alpha-glucosidase (EC 3.2.1.20): alpha-1,3-glucosidase (EC 3.2.1.84); sucrase-isomaltase (EC 3.2.1.48) (EC 3.2.1.10); alpha-xylosidase (EC 3.2.1.-); alpha-glucan lyase (EC 4.2.2.13); isomaltosyltransferase (EC 2.4.1.-); alpha-N-acetylgalactosaminidase (EC 3.2.1.49); stachyose synthase (EC 2.4.1.67); raffinose synthase (EC 2.4.1.82); alpha-mannosidase (EC 3.2.1.24); alpha-mannosidase (EC 3.2.1.114); beta-1,3-xylosidase (EC 3.2.1.-);alpha-L-arabinofuranosidase (EC 3.2.1.55); arabinanase (EC 3.2.1.99); galactan 1,3-beta-galactosidase (EC 3.2.1.145); chitinase (EC 3.2.1.14); alpha-L-arabinofuranosidase (EC 3.2.1.55); trehalase (EC 3.2.1.28); maltose phosphorylase (EC 2.4.1.8); trehalose phosphorylase (EC 2.4.1.64); kojibiose phosphorylase (EC 2.4.1.230); alpha-glucuronidase (EC 3.2.1.139); xylan alpha-1,2-glucuronosidase (EC_{—}3.2.1.131); amylomaltase (EC 2.4.1.25); endo-beta-N-acetylglucosaminidase (EC 3.2.1.96); mycodextranase (EC 3.2.1.61); alpha-1,3-glucanase (EC 3.2.1.59); d-4,5 unsaturated beta-glucuronyl hydrolase (EC 3.2.1.-); cellobiose phosphorylase (EC 2.4.1.20); cellodextrin phosphorylase (EC 2.4.1.49); chitobiose phosphorylase (EC 2.4.1.-); cyclic beta-1,2-glucan synthase (EC 2.4.1.-); alpha-1,2-L-fucosidase (EC 3.2.1.63); alpha-L-fucosidase (EC 3.2.1.51); unsaturated rhamnogalacturonyl hydrolase (EC 3.2.1.-); alpha-L-rhamnosidase (EC 3.2.1.40); lacto-N-biose phosphorylase or galacto-N-biose phosphorylase (EC 2.4.1.211); or a combination thereof.

Disclosed herein are isolated microorganisms comprising a genetic modification to produce higher levels of one or more cellulase enzymes in the presence of an inhibitory molecule. The inhibitory molecule can be a saccharide (e.g., a monosaccharide or polysaccharide). The inhibitory molecule can be a catabolite produced by one or more cellulases. The inhibitory molecule can be xylose. The inhibitory molecule can be glucose or a glucose analog (e.g., 2-deoxyglucose). In one embodiment, the microorganism constitutively expresses the cellulase enzymes. In one embodiment, cellulase expression in the microorganism is not subject to feedback inhibition. In one embodiment, the genetic modification comprises a mutation in one or more genes. Any number of genes can be mutated in a microorganism to deregulate cellulases; for example, 1, 2, 3, 4, 5, 6, 7, 8, 9, 10, 11, 12, 13, 14, 15, 16, 17, 18, 19, 20, or more genes can be mutated. Any individual mutation can be a point mutation (e.g., a synonymous mutation, a missense mutation, a nonsense mutation), a deletion (e.g., a frame-shift mutation), an insertion (e.g., a splice site mutation, a frame-shift mutation), an amplification (e.g., a duplication of one or more genes), a translocation, a transversion, or a micro deletion. The mutation can be a gain of function mutation. The mutation in a gene can increase an activity, stability, or expression level of an encoded protein. The mutation can be a loss of function mutation. The mutation in a gene can decrease an activity, stability, or expression level of an encoded protein. In one embodiment, the mutated gene(s) encode a propionyl-CoA carboxylase, a two component AraC family transcriptional regulator, a ROK family glucokinase, a homolog of Cphy_{—}3487, a dihydrolipoamide dehydrogenase, a binding-protein-dependent transport systems inner membrane component, an ABC transporter related protein, a homolog of Cphy_{—}0056, a TetR family transcriptional regulator, an AraC-like protein, a glycosyl transferase 36, a diaminopimelate epimerase, an oxidoreductase domain-containing protein, a homolog of Cphy2965, a desulfoferrodoxin ferrous iron-binding region, a homolog of Cphy_{—}1063, an AraC family transcriptional regulator, a phage tape measure protein, a D-isomer specific 2-hydroxyacid dehydrogenase NAD-binding protein, an HD superfamily phosphohydrolase-like protein, or a combination thereof. In one embodiment, the microorganism comprises a mutation in a gene encoding a ROK family glucokinase. In one embodiment, the mutation alters the carbohydrate biding pocket of the ROK family glucokinase. In one embodiment, the altered carbohydrate binding pocket has a lower affinity for a carbohydrate. In one embodiment, the altered carbohydrate binding pocket has a higher affinity for a carbohydrate. In one embodiment, the carbohydrate is a disaccharide. In one embodiment, the carbohydrate is a monosaccharide. The carbohydrate can be a hexose or a pentose. The carbohydrate can be glucose, fructose, xylose, sucrose, or a combination thereof. In one embodiment, the microorganism comprises a mutation in a gene encoding a two component AraC famly transcriptional regulator. The AraC family transcriptional regulator can be a repressor, an activator, or both. In one embodiment, the AraC family transcriptional regulator is self-regulating. In one embodiment, the AraC family transcriptional regulator regulates the expression of an operon. The operon can contain genes that encode proteins involved in carbohydrate metabolism. The operon can contain genes that encode ABC transport proteins. The operon can contain genes that encode for cellulases. The microorganism can comprise a mutated gene that encodes a propionyl-CoA carboxylase. The propionyl-CoA carboxylase can be an activator of a glucokinase. In one embodiment, the mutated propionyl-CoA carboxylase can decrease the activity of the glucokinase. In one embodiment, the microorganism comprises mutations in a ROK family glucokinase, a two component AraC famly transcriptional regulator, and a propionyl-CoA carboxylase. In one embodiment, the microorganism comprises mutations in a ROK family glucokinase, a two component AraC famly transcriptional regulator, a propionyl-CoA carboxylase, a homolog of Cphy_{—}3487, and a dihydrolipoamide dehydrogenase. In one embodiment, the microorganism comprises mutations in a ROK family glucokinase, a two component AraC famly transcriptional regulator, a propionyl-CoA carboxylase, a binding-protein-dependent transport systems inner membrane component, an ABC transporter related protein, and a homolog of Cphy_{—}0056. In one embodiment, the microorganism comprises mutations in a ROK family glucokinase, a two component AraC famly transcriptional regulator, a propionyl-CoA carboxylase, a TetR family transcriptional regulator, and an AraC-like protein. In one embodiment, the microorganism comprises mutations in a ROK family glucokinase, a two component AraC family transcriptional regulator, a propionyl-CoA carboxylase, an HD superfamily phosphohydrolase-like protein, a diaminopimelate epimerase, an oxidoreductase domain-containing protein, a homolog of Cphy_{—}2965, a desulfoferrodoxin ferrous iron-binding region, a homolog of Cphy_{—}1063, an AraC family transcriptional regulator, a phage tape measure protein, a D-isomer specific 2-hydroxyacid dehydrogenase NAD-binding protein, and an HD superfamily phosphohydrolase-like protein.

Glucose-6-phosphate can be a signal molecule that shuts down or represses the expression of genes that encode cellulases. In one embodiment, the microorganism with deregulated cellulases produces less glucose-6-phosphate. In one embodiment, cellulase expression in the genetically modified microorganism is less sensitive or insensitive to glucose-6-phosphate.

Increased Production of Fermentation End-Products

In one aspect, disclosed herein are isolated microorganisms that can produce one or more fermentation end products from a biomass that comprise a genetic modification to produce higher levels of cellulase enzymes in the presence of an inhibitory molecule, methods of producing a fermentation end-product with the microorganism, and plants for the production of fermentation end products with the microorganism wherein the microorganism comprising the genetic modification produces more of the fermentation end-product than a microorganism of the same type without genetic modification. For example, the genetically modified microorganism can produce about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or more of a fermentation end-product in comparison to an unmodified microorganism of the same species. In one embodiment, the genetically modified microorganism produces a greater saccharification yield from the biomass than a non-genetically modified microorganism of the same type; for example, about 1-300%, 1-250%, 1-200%, 1-150%, 1-100%, 1-90%, 1-80%, 1-70%, 1-60%, 1-50%, 1-40%, 1-30%, 1-20%, 1-10%, 10-300%, 10-250%, 10-200%, 10-150%, 10-100%, 10-90%, 10-80%, 10-70%, 10-60%, 10-50%, 10-40%, 10-30%, 10-20%, 20-300%, 20-250%, 20-200%, 20-150%, 20-100%, 20-90%, 20-80%, 20-70%, 20-60%, 20-50%, 20-40%, 20-30%, 30-300%, 30-250%, 30-200%, 30-150%, 30-100%, 30-90%, 30-80%, 30-70%, 30-60%, 30-50%, 30-40%, 40-300%, 40-250%, 40-200%, 40-150%, 40-100%, 40-90%, 40-80%, 40-70%, 40-60%, 40-50%, 50-300%, 50-250%, 50-200%, 50-150%, 50-100%, 50-90%, 50-80%, 50-70%, 50-60%, 60-300%, 60-250%, 60-200%, 60-150%, 60-100%, 60-90%, 60-80%, 60-70%, 70-300%, 70-250%, 70-200%, 70-150%, 70-100%, 70-90%, 70-80%, 80-300%, 80-250%, 80-200%, 80-150%, 80-100%, 80-90%, 90-300%, 90-250%, 90-200%, 90-150%, 90-100%, 100-300%, 100-250%, 100-200%, 100-150%, 150-300%, 150-250%, 150-200%, 200-300%, 200-250%, 250-300%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 220%, 240%, 260%, 280%, 300%, or more saccharification yield. In one embodiment, the saccharification yield produced by the microorganism with deregulated cellulases is about 80% to 100% of the theoretical yield; for example, about 80-100%, 80-90%, 90-100%, 80%, 81%, 82%, 83%, 84%, 85%, 86%, 87%, 88%, 89%, 90%, 91%, 92%, 93%, 94%, 95%, 96%, 97%, 98%, 99%, or 100% of the theoretical saccharification yield. The fermentation end-product can be an alcohol. The alcohol can be ethanol. In one embodiment, the biomass comprises cellulose, hemicellulose, lignocellulose, or a combination thereof. In one embodiment, the biomass comprises hemicellulosic or lignocellulosic material. In one embodiment, the inhibitory molecule is glucose or a glucose analog (e.g., 2-deoxyglucose (DoG)).

Measuring Increased Cellulase Synthesis in Deregulated Cellulase Mutants

Any useful method can be used to measure the increased synthesis of cellulases in the presence of an inhibitory molecule by a genetically modified microorganism with deregulated cellulases. The inhibitor can be glucose or a glucose analog (e.g., 2-deoxyglucose). Increased synthesis of cellulases in the presence of an inhibitor molecule can be measured under initial conditions where the only carbon sources are oligosaccharides. The oligosaccharide can be any carbohydrate having two or more subunits; for example, cellobiose, lactose, sucrose, maltose, cellulose, hemicellulose, lignocellulose, xylan, callose, chrysolaminarin, arabinoxylan, mannan, fucoidan, galactomannan, etc. The size of the clearing zone (e.g., as measured by Congo-Red staining) around a colony grown on phosphohoric acid-swollen cellulose (PASC) agar plates in the presence of an inhibitor molecule can be indicative of increased synthesis of cellulases. The size of the clearing zone can be measured, for example, according to the radius of the clearing zone, the diameter of the clearing zone, the area of the clearing zone, or the volume of the clearing zone. A microorganism genetically modified to have deregulated cellulase expression (e.g., a microorganism that synthesizes more cellulases in the presence of an inhibitor molecule) can have a clearing zone that is about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 5-20, 5-15, 5-10, 5-9, 5-8, 5-7, 5-6, 6-20, 6-15, 6-10, 6-9, 6-8, 6-7, 7-20, 7-15, 7-10, 7-9, 7-8, 8-20, 8-15, 8-10, 8-9, 9-20, 9-15, 9-10, 10-20, 10-15, or 15-20 times larger than a clearing zone of a microorganism of the same species that is not genetically modified; for example, the clearing zone of a genetically modified microorganism can be about 1.1, 1.2, 1.3, 1.4, 1.5, 1.6, 1.7, 1.8, 1.9, 2, 2.2, 2.4, 2.6, 2.8, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 times larger. Clearing zones can be measured, for example, after 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours of growth under suitable conditions for the microorganism. Clearing zones can be measured after 1, 2, 3, 4, 5, 6, 7, 8, 9, or 10 days of growth under suitable conditions. In one embodiment, clearing zones can be measured without the use of the inhibitor molecule. Clearing zones can be measured after between about 1 hour and 10 days of growth under suitable conditions; for example, between about 1 hour and 10 days, 1 hour and 5 days, 1 hour and 2 days, 12 hours and 10 days, 12 hours and 5 days, 12 hours and 2 days, 1 day and 10 days, 1 day and 5 days, 1 day and 4 days, 1 day and 3 days, or 1 day and 2 days. In one embodiment, clearance zones are measured after about 2 days of growth under appropriate conditions.

Increased synthesis of cellulases in the presence of an inhibitor molecule can be measured in a fluorescent cellulase activity assay. The fluorescent cellulase activity assay can be performed according to Example 3. The fluorescent cellulase activity assay can be performed with or without the use of an inhibitor molecule (e.g., glucose or a glucose analog, e.g., 2-deoxyglucose). In one embodiment, the fluorescent cellulase activity assay comprises 4-methylumbilliferone (4Mu) substrates (e.g., glucopyranoside, cellobioside, etc.). The fluorescent cellulase activity assay can be performed with or without an inhibitor of β-galactosidase (e.g., gluconolactone). The fluorescent cellulase activity can be measured (e.g., by emission at 465 nM with excitation at 360 nM) at multiple time points; for example, 1, 2, 3, 4, 5, 6, 7, 8, 9, 10, 11, 12, 13, 14, 15, 16, 17, 18, 19, 20, or more timepoints. In one embodiment, two time points are measured. The first time point can be at time zero. Successive time points can be measured at any time; for example, after between about 1 and 48 hours (e.g., about 1-48, 1-36, 1-24, 1-18, 1-12, 1-6, 6-48, 6-36, 6-24, 6-18, 6-12, 12-48, 12-36, 12-24, 12-18, 18-48, 18-36, 18-24, 24-48, 24-36, or 36-48 hours); for example, after about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours or longer. In one embodiment, measurements in the fluorescent cellulase assay are taken at 0 hours and 18 hours. Measurements in a cellulase assay (e.g. emission at 465 nM with excitation at 360 nM) can be normalized by cell density. Normalization by cell density can enable comparisons between cultures at different densities. Cellulase activity for a genetically modified microorganism in a fluorescent cellulase activity assay can be measured in terms of fold increase in comparison to a non-genetically modified microorganism of the same species. A fold increase can be defined as an increase of more than two standard deviations. A microorganism comprising a genetic modification that enables said microorganism to produce more cellulases in the presence of an inhibitory molecule can exhibit between about 1 and 20 fold, or more, increases in cellulase activity in the fluorescent cellulase activity assay in comparison to a microorganism of the same species without said genetic modification; for example, about 1-20, 1-15, 1-10, 1-8, 1-6, 1-5, 1-4, 1-3, 1-2, 2-20, 2-15, 2-10, 2-8, 2-6, 2-5, 2-4, 2-3, 3-20, 3-15, 3-10, 3-8, 3-6, 3-5, 3-4, 4-20, 4-15, 4-10, 4-8, 4-6, 4-5, 5-20, 5-15, 5-10, 5-8, 5-6, 6-20, 6-15, 6-10, 6-8, 8-20, 8-15, 8-10, 10-20, 10-15, 15-20, 1, 2, 3, 4, 5, 6, 7, 8, 9, 10, 11, 12, 13, 14, 15, 16, 17, 18, 19, or 20 fold, or more, increases in cellulase activity in the fluorescent cellulase activity assay.

Increased synthesis of cellulases in the presence of an inhibitor molecule by a genetically modified microorganism can be evidenced by increased levels of cellulase encoding mRNA or increased levels of cellulase protein measured after growth or fermentation in the presence of an inhibitor molecule, as compared to a non-genetically modified microorganism of the same species. The inhibitor molecule can be glucose or a glucose analog (e.g., 2-deoxyglucose). Increased levels of cellulase encoding mRNA can be measured using any useful technology, for example, northern blotting, reverse transcription quantitative Polymerase Chain Reaction (RT-qPCR), DNA microarray, fluorescent in situ hybridization, etc. Increase levels of cellulase protein can be measured using any useful technology, for example, western blotting, fluorescence-based assays (e.g., expression of a cellulase fused to a fluorescent protein, immunocytochemistry, etc.), enzyme-linked immunosorbent assays, etc. Protein and/or mRNA levels can be measured during fermentation of a biomass by the genetically modified microorganism; for example, aliquots of a fermentation culture can be taken at one or more time-points during fermentation (e.g., 1, 2, 3, 4, 5, 6, 7, 8, 9, 10, 11, 12, 13, 14, 15, 16, 17, 18, 19, 20, or more aliquots) and used to isolate protein or mRNA that can be detected using any techniques disclosed herein. Protein and/or mRNA levels can be determined in conjunction with any other methods to test for increased cellulase expression disclosed herein; for example, mRNA and/or protein can be measured in a colony following a clearance assay, mRNA and/or protein can be measured from an aliquot sampled during or following a fluorescent cellulase activity assay, etc. A genetically modified microorganism with deregulated cellulase expression (e.g., a genetically modified microorganism that synthesizes more cellulases in the presence of an inhibitory molecule) can be characterized as having between about a 1 and a 100 fold increase in the levels of mRNA encoding a cellulase and/or cellulase protein in comparison to a non-genetically modified microorganism of the same species. The fold increase in mRNA or protein levels can be about 1-100, 1-75, 1-50, 1-40, 1-30, 1-20, 1-10, 1-5, 1-4, 1-3, 1-2, 5-100, 5-75, 5-50, 5-40, 5-30, 5-20, 5-10, 10-100, 10-75, 10-50, 10-40, 10-30, 10-20, 20-100, 20-75, 20-50, 20-40, 20-30, 30-100, 30-75, 30-50, 30-40, 40-100, 40-75, 40-50, 50-100, 50-75, 75-100, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, or 100 times, or more, when comparing a genetically modified microorganism to a non-genetically modified microorganism of the same species.

Isolated Microorganisms

An isolated microorganism comprising a genetic modification to produce or synthesize higher levels of cellulase enzymes in the presence of an inhibitory molecule can be any microorganism wherein cellulase expression is subject to feedback inhibition. The isolated microorganism can be a bacteria, a yeast, or another fungus. Examples of yeast include, but are not limited to, species found in the genus Ascoidea, Brettanomyces, Candida, Cephaloascus, Coccidiascus, Dipodascus, Eremothecium, Galactomyces, Kluyveromyces, Pichia, Saccharomyces, Schizosaccharomyces, Sporopachydermia, Torulaspora, Yarrowia, or Zygosaccharomyces; for example, Ascoidea rebescens, Brettanomyces anomalus, Brettanomyces bruxellensis, Brettanomyces claussenii, Brettanomyces custersianus, Brettanomyces lambicus, Brettanomyces naardenensis, Brettanomyces nanus, Candida albicans, Candida ascalaphidarum, Candida amphixiae, Candida antarctica, Candida argentea, Candida atlantica, Candida atmosphaerica, Candida blattae, Candida carpophila, Candida cerambycidarum, Candida chauliodes, Candida corydali, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida krusei, Candida lusitaniae, Candida lyxosophila, Candida maltosa, Candida marina, Candida membranifaciens, Candida milleri, Candida oleophila, Candida oregonensis, Candida parapsilosis, Candida quercitrusa, Candida rugosa, Candida sake, Candida shehatea, Candida temnochilae, Candida tenuis, Candida tropicalis, Candida tsuchiyae, Candida sinolaborantium, Candida sojae, Candida subhashii, Candida viswanathii, Candida utilis, Cephaloascus fragrans, Coccidiascus legeri, Dypodascus albidus, Eremothecium cymbalariae, Galactomyces candidum, Galactomyces geotrichum, Kluyveromyces aestuarii, Kluyveromyces africanus, Kluyveromyces bacillisporus, Kluyveromyces blattae, Kluyveromyces dobzhanskii, Kluyveromyces hubeiensis, Kluyveromyces lactis, Kluyveromyces lodderae, Kluyveromyces marxianus, Kluyveromyces nonfermentans, Kluyveromyces piceae, Kluyveromyces sinensis, Kluyveromyces thermotolerans, Kluyveromyces waltii, Kluyveromyces wickerhamii, Kluyveromyces yarrowii, Pichia anomola, Pichia heedii, Pichia guilliermondii, Pichia kluyveri, Pichia membranifaciens, Pichia norvegensis, Pichia ohmeri, Pichia pastoris, Pichia subpelliculosa, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus, Schizosaccharomyces cryophilus, Schizosaccharomyces japonicus, Schizosaccharomyces octosporus, Schizosaccharomyces pombe, Sporopachydermia cereana, Sporopachydermia lactativora, Sporopachydermia quercuum, Torulaspora delbrueckii, Torulaspora franciscae, Torulaspora globosa, Torulaspora pretoriensis, Yarrowia lipolytica, Zygosaccharomyces bailii, Zygosaccharomyces bisporus, Zygosaccharomyces cidri, Zygosaccharomyces fermentati, Zygosaccharomyces florentinus, Zygosaccharomyces kombuchaensis, Zygosaccharomyces lentus, Zygosaccharomyces mellis, Zygosaccharomyces microellipsoides, Zygosaccharomyces mrakii, Zygosaccharomyces pseudorouxii, or Zygosaccharomyces rouxii. Examples of bacteria include, but are not limited to, any bacterium found in the genus of Butyrivibrio, Ruminococcus, Eubacterium, Bacteroides, Acetivibrio, Caldibacillus, Acidothermus, Cellulomonas, Curtobacterium, Micromonospora, Actinoplanes, Streptomyces, Thermobifida, Thermomonospora, Microbispora, Fibrobacter, Sporocytophaga, Cytophaga, Flavobacterium, Achromobacter, Xanthomonas, Cellvibrio, Pseudomonas, Myxobacter, Escherichia, Klebsiella, Thermoanaerobacterium, Thermoanaerobacter, Geobacillus, Saccharococcus, Paenibacillus, Bacillus, Caldicellulosiruptor, Anaerocellum, Anoxybacillus, Zymomonas, Clostridium; for example, Butyrivibrio fibrisolvens, Ruminococcus flavefaciens, Ruminococcus succinogenes, Ruminococcus albus, Eubacterium cellulolyticum, Bacteroides cellulosolvens, Acetivibrio cellulolyticus, Acetivibrio cellulosolvens, Caldibacillus cellulovorans, Bacillus circulans, Acidothermus cellulolyticus, Cellulomonas cartae, Cellulomonas cellasea, Cellulomonas cellulans, Cellulomonas fimi, Cellulomonas flavigena, Cellulomonas gelida, Cellulomonas iranensis, Cellulomonas persica, Cellulomonas uda, Curtobacterium falcumfaciens, Micromonospora melonosporea, Actinoplanes aurantiaca, Streptomyces reticuli, Streptomyces alboguseolus, Streptomyces aureofaciens, Streptomyces cellulolyticus, Streptomyces flavogriseus, Streptomyces lividans, Streptomyces nitrosporeus, Streptomyces olivochromogenes, Streptomyces rochei, Streptomyces thermovulgaris, Streptomyces viridosporus, Thermobifida alba, Thermobifida fusca, Thermobifida cellulolytica, Thermomonospora curvata, Microbispora bispora, Fibrobacter succinogenes, Sporocytophaga myxococcoides, Cytophaga sp., Flavobacterium johnsoniae, Achromobacter piechaudii, Xanthomonas sp., Cellvibrio vulgaris, Cellvibrio fulvus, Cellvibrio gilvus, Cellvibrio mixtus, Pseudomonas fluorescens, Pseudomonas mendocina, Myxobacter sp. AL-1, Escherichia albertii, Escherichia blattae, Escherichia coli, Escherichia fergusonii, Escherichia hermannii, Escherichia vulneris, Klebsiella granulomatis, Klebsiella oxytoca, Klebsiella pneumonia, Klebsiella terrigena, Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, Anaerocellum thermophilum, Clostridium thermocellum, Clostridium cellulolyticum, Clostridium straminosolvens, Clostridium acetobutylicum, Clostridium aerotolerans, Clostridium beijerinckii, Clostridium bifermentans, Clostridium botulinum, Clostridium butyricum, Clostridium cadaveric, Clostridium chauvoei, Clostridium clostridioforme, Clostridium colicanis, Clostridium difficile, Clostridium fallax, Clostridium formicaceticum, Clostridium histolyticum, Clostridium innocuum, Clostridium ljungdahlii, Clostridium laramie, Clostridium lavalense, Clostridium novyi, Clostridium oedematiens, Clostridium paraputrificum, Clostridium perfringens, Clostridium phytofermentans (including NRRL B-50364 or NRRL B-50351), Clostridium piliforme, Clostridium ramosum, Clostridium scatologenes, Clostridium septicum, Clostridium sordellii, Clostridium sporogenes, Clostridium sp. Q.D (such as NRRL B-50361, NRRL B-50362, or NRRL B-50363), Clostridium tertium, Clostridium tetani, Clostridium tyrobutyricum, Clostridium thermobutyricum, Zymomonas mobilis or variants thereof (e.g. C. phytofermentans Q.12 or C. phytofermentans Q.13). In one embodiment, the microorganism can be Clostridium phytofermentans Q.17 (hereinafter “Q.17”), Clostridium phytofermentans Q.18 (hereinafter “Q.18”), Clostridium phytofermentans Q.19 (hereinafter “Q.19”), or Clostridium phytofermentans Q.20 (hereinafter “Q.20”), having the NRRL patent deposit designations NRRL B-50447, NRRL B-50448, NRRL B-50449, and NRRL B-50450, respectively. In one embodiment, the microorganism is a bacteria. In one embodiment, the bacterium is a Clostridium strain. The Clostridium strain can be Clostridium thermocellum, Clostridium beijerinickii, Clostridium acetobutylicum, Clostridium cellulolyticum, Clostridium tyrobutyricum, or Clostridium thermobutyricum. In one embodiment, the Clostridium species is a Clostridium phytofermentans strain. In one embodiment, the Clostridium phytofermentans strain can be, for example, Clostridium phytofermentans Q.8, Clostridium phytofermentans Q.33, or Clostridium phytofermentans Q.32. In one embodiment, the Clostridium species is Clostridium Q.D. In one embodiment, the microorganism is Clostridium phytofermentans, Clostridium Q.D, or a variant thereof. In one embodiment, the microorganism can be Clostridium phytofermentans Q.17, Q.18, Q.19, or Q.20.

The isolated microorganism comprising a genetic modification to produce or synthesize higher levels of cellulase enzymes in the presence of an inhibitory molecule can further comprise a genetic modification in a gene or pathway that is not involved in cellulase regulation. In one embodiment, a microorganism can be genetically modified to express one or more polypeptides capable of neutralizing a toxic by-product or inhibitor, which can result in enhanced end-product production in yield and/or rate of production. Examples of modifications include chemical or physical mutagenesis, directed evolution, or genetic alteration to enhance enzyme activity of endogenous proteins, introducing one or more heterogeneous nucleic acid molecules into a host microorganism to express a polypeptide not otherwise expressed in the host, modifying physical and chemical conditions to enhance enzyme function (e.g., modifying and/or maintaining a certain temperature, pH, nutrient concentration, or biomass concentration), or a combination of one or more such modifications.

The isolated microorganism comprising a genetic modification to produce or synthesize higher levels of cellulase enzymes in the presence of an inhibitory molecule can be used as a biocatalyst in the biofuel or biochemical industry. In one embodiment, the microorganism can hydrolyze a biomass comprising oligosaccharides (e.g., cellulose, hemicellulose, lignocellulose, cellobiose, xylan, etc.). In one embodiment, the microorganism can ferment a biomass. In one embodiment, the microorganism can hydrolyze and ferment a biomass. A microorganism that both hydrolyzes and ferments biomass efficiently is Clostridium phytofermentans (ISDg^{T}, American Type Culture Collection 700394^{T}). See U.S. Pat. No. 7,682,811 B2, which is herein incorporated by reference in its entirety. In another embodiment, a microorganism is genetically modified or subject to mutation to modulate the activity of a metabolic pathway to produce energy-rich products from the conversion of carbohydrates. See, e.g., Lynd, et al. Curr. Opinion Biotechnol. 16:577-583 (2005). In another embodiment, strain development provides processes by which new organisms can be derived and screened that possess the attributes for enhanced yields on industrial scales.

In another embodiment, processes that identify such organisms can be useful in screening of other organisms that show the same basic characteristics. Routine procedures for microbial species identification rely on examination of the colony (pigmentation of the surface and reverse sides, topography, texture, and rate of growth) and microscopic morphology (size and shape of cells and spores) and staining (gram-positive v. gram-negative). Further identification characteristics include nutritional requirements (vitamins and amino acids) and temperature tolerance, as well as product production, etc. Morphological and physiological characteristics can frequently vary; in fact, the phenotypic features can be easily influenced by outside factors such as temperature variation, medium, and chemotherapy and therefore strain identification is often difficult. In one embodiment, provided herein are isolated Gram-positive Clostridium phytofermentans bacterial strains, wherein the bacteria are obligate anaerobic, mesophilic, cellulolytic organisms that can use polysaccharides as a sole carbon source and can oxidize glucose into ethanol or one or more organic acids as its fermentation product. In another embodiment, provided herein are isolated bacteria designated Clostridium phytofermentans Q.17, Clostridium phytofermentans Q.18, Clostridium phytofermentans Q.19, or Clostridium phytofermentans Q.20, having the NRRL patent deposit designations NRRL B-50447, NRRL B-50448, NRRL B-50449, and NRRL B-50450, respectively. As used herein, “obligate” means required or compulsory. As used herein, a “mesophilic” is a bacterium that preferentially ferments a carbon source at about 30-40° C. Q.17, Q.18, Q.19 and Q.20 consist of motile rods that form terminal spores.

In one embodiment, the isolated bacteria Q.17, Q.18, Q.19 or Q.20 are obligate anaerobic mesophiles that demonstrate increased cellulase activity compared to their parent strain, Clostridium phytofermentans Q.8, and can ferment biomass or carbonaceous material into ethanol, organic acids and other fermentation end products. For example, these bacteria can degrade cellulose and/or xylose into ethanol and acetic acid, lactic acid, aspartic acid, malic acid or glutamic acid.

### Definitions

In this specification and in the claims which follow, reference will be made to a number of terms which shall be defined to have the following meanings. Unless characterized otherwise, technical and scientific terms used herein have the same meaning as commonly understood by one of ordinary skill in the art.

As used in the specification and the appended claims, the singular forms “a,” “an” and “the” include plural referents unless the context clearly dictates otherwise. Thus, for example, reference to “a purified polypeptide” includes mixtures of two or more purified polypeptides.

The term “comprising” as used herein is synonymous with “including,” “containing,” or “characterized by,” and is inclusive or open-ended and does not exclude additional, unrecited elements or method steps.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. “About” means a referenced numeric indication plus or minus 10% of that referenced numeric indication. For example, the term about 4 would include a range of 3.6 to 4.4.

All numbers expressing quantities of ingredients, reaction conditions, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

“Optional” or “optionally” means that the subsequently described event or circumstance may or may not occur, and that the description includes instances where said event or circumstance occurs and instances where it does not. For example, the phrase “the medium can optionally contain glucose” means that the medium may or may not contain glucose as an ingredient and that the description includes both media containing glucose and media not containing glucose.

It is understood that as discussed herein, the terms “similar” or “similarity” mean the same thing as “homology” and “identity.” Thus, for example, if the use of the word homology is used to refer to two non-natural sequences, it is understood that this is not necessarily indicating an evolutionary relationship between these two sequences, but rather is looking at the similarity or relatedness between their nucleic acid or amino acid sequences. Many of the methods for determining similarity between two evolutionarily related molecules are routinely applied to any two or more nucleic acids or polypeptides for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of similarity, or homology, to specific known sequences. In general, variants of nucleic acids and polypeptides herein disclosed can typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent similarity, or homology, to the stated sequence or the native sequence. Those of skill in the art readily understand how to determine the similarity of two polypeptides or nucleic acids. For example, the similarity can be calculated after aligning the two sequences so that the similarity is at its highest level.

Another way of calculating similarity, or homology, can be performed by published algorithms. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. U.S.A. 85: 2444 (1988); by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (www.ncbi.nlm nih.gov/blast/bl2seq/b12.html)); or by inspection.

The same types of similarity, or homology, can be obtained for nucleic acids by, for example, the algorithms disclosed in Zuker, M. Science 244:48-52, 1989; Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if similarity is found with at least one of these methods, the sequences would be said to have the stated similarity.

For example, as used herein, a sequence recited as having a particular percent similarity to another sequence refers to sequences that have the recited similarity as calculated by any one or more of the calculation methods described above. For example, a first sequence has 80 percent similarity, as defined herein, to a second sequence if the first sequence is calculated to have 80 percent similarity to the second sequence using the Zuker calculation method even if the first sequence does not have 80 percent similarity to the second sequence as calculated by any of the other calculation methods. As another example, a first sequence has 80 percent similarity, as defined herein, to a second sequence if the first sequence is calculated to have 80 percent similarity to the second sequence using both the Zuker calculation method and the Pearson and Lipman calculation method even if the first sequence does not have 80 percent similarity to the second sequence as calculated by the Smith and Waterman calculation method, the Needleman and Wunsch calculation method, the Jaeger calculation methods, or any of the other calculation methods. As yet another example, a first sequence has 80 percent similarity, as defined herein, to a second sequence if the first sequence is calculated to have 80 percent similarity to the second sequence using each of the calculation methods (although, in practice, the different calculation methods will often result in different calculated similarity percentages).

As used herein, the term “nucleic acid” refers to single or multiple stranded molecules which may be DNA or RNA, or any combination thereof, including modifications to those nucleic acids. The nucleic acid can represent a coding strand or its complement, or any combination thereof. Nucleic acids can be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in Sambrook et al. (2001) Molecular Cloning—A Laboratory Manual (3rd ed.) Vol. 1-3, Cold Spring Harbor Laboratory, Cold Spring Harbor Press, NY, (Sambrook).

The nucleic acid can be detected with a probe capable of hybridizing to the nucleic acid of a cell or a sample. This probe can be a nucleic acid comprising the nucleotide sequence of a coding strand or its complementary strand or the nucleotide sequence of a sense strand or antisense strand, or a fragment thereof. The nucleic acid can comprise the nucleic acid of the bacterial genome, or fragments thereof. In one embodiment, the probe can be either DNA or RNA and can bind either DNA or RNA, or both, in the biological sample.

As used herein, the term “nucleic acid probe” refers to a nucleic acid fragment that selectively hybridizes under stringent conditions with a nucleic acid comprising a nucleic acid set forth in a sequence listed herein. This hybridization can be specific. The degree of complementarity between the hybridizing nucleic acid and the sequence to which it hybridizes should be at least enough to exclude hybridization with a nucleic acid encoding an unrelated protein.

As used herein, the term “primer” refers to a single-stranded oligonucleotide that is extended by covalent bonding of nucleotide monomers during amplification or polymerization of a nucleic acid molecule.

“Stringent conditions” refers to the washing conditions used in a hybridization protocol. In general, the washing conditions should be a combination of temperature and salt concentration chosen so that the denaturation temperature is approximately 5° C. to 20° C. below the calculated Tm of the nucleic acid hybrid under study. In one embodiment, the denaturation temperature is approximately 5° C., 6° C., 7° C., 8° C., 9°-C. 10° C. 11° C. 12° C. 13° C. 14° C. 15° C. 16° C. 17° C. 18° C. 19° C. or 20° C. below the calculated Tm of the nucleic acid hybrid under study. The temperature and salt conditions are readily determined empirically in preliminary experiments in which samples of reference DNA immobilized on filters are hybridized to the probe or polypeptide-coding nucleic acid of interest and then washed under conditions of different stringencies. The Tm of such an oligonucleotide can be estimated by allowing 2° C. for each A or T nucleotide, and 4° C. for each G or C. For example, an 18 nucleotide probe of 50% G+C would, therefore, have an approximate Tm of 54° C. Stringent conditions are known to one of skill in the art. See, for example, Sambrook et al. (2001). The following is an exemplary set of hybridization conditions and is not limiting:

Very High Stringency

Hybridization: 5×SSC at 65° C. for 16 hours. Wash twice: 2×SSC at room temperature (RT) for 15 minutes each. Wash twice: 0.5×SSC at 65° C. for 20 minutes each.

High Stringency

Hybridization: 5×-6×SSC at 65° C.-70° C. for 16-20 hours. Wash twice: 2×SSC at RT for 5-20 minutes each. Wash twice: 1×SSC at 55° C.-70° C. for 30 minutes each.

Low Stringency

Hybridization: 6×SSC at RT to 55° C. for 16-20 hours. Wash at least twice: 2×-3×SSC at RT to 55° C. for 20-30 minutes each.

In one embodiment, provided is a method of fermenting a carbonaceous material, comprising contacting the carbonaceous material with an effective, fermenting amount of an isolated Gram-positive bacterium, wherein the bacterium is an anaerobic, obligate mesophile, wherein the bacterium produces increased cellulase activity compared to its parent strain Q.8, and can use polysaccharides as a sole carbon source and can reduce acetaldehyde into ethanol, whereby contacting the carbonaceous material with the bacterium ferments the carbonaceous material. As used herein, an “effective amount” is within the knowledge of one skilled in the art. Various methods are known by which a person of skill can determine the amount of bacteria required to effectively ferment a carbonaceous material, e.g., biomass, of interest. The carbonaceous materials can be any one or more of the materials disclosed herein. In one aspect, the bacterial strain is Q.17. In another aspect, the bacterial strain is Q.18. In a further aspect, the bacterial strain is Q.19. In yet another aspect, the bacterial strain is Q.20.

In one embodiment, the contacting step of the disclosed method occurs at a pH of from about 5.0 to about 7.5. In one embodiment, the contacting step occurs at a pH of from about 6.0 to about 6.5. The contacting step can occur at a pH of about 5, 6, 7, or 8. In one embodiment, the method disclosed is carried out at a temperature from about 30° C. to about 40° C. In one embodiment, the disclosed method is carried out at a temperature from about 35° C. to about 37° C. Additional temperatures at which the disclosed method can be carried out are 31° C., 32° C., 33° C., 34° C., 35° C., 36° C., 37° C., 38° C., and 39° C.

In one embodiment, disclosed herein are methods of growing an isolated Gram-positive bacterium, designated Q.17, Q.18, Q.19 or Q.20 and deposited under NRRL Accession Nos. B-50447, NRRL B-50448, NRRL 50449, or NRRL 50450, respectively. In another embodiment, the bacterial strains, designated Q.17, Q.18, Q.19 or Q.20, and deposited under NRRL Accession Nos. B-50447, NRRL B-50448, NRRL 50449, or NRRL 50450, respectively are provided. In one embodiment, the bacterium is an anaerobic, obligate mesophile, wherein the bacterium can use cellulose as a sole carbon source and can oxidize acetaldehyde into ethanol, comprising culturing the bacterium at a temperature and on a medium effective to promote growth of the bacterium. The bacterium can grow at a temperature from about 30° C. to about 40° C. In one aspect, the bacterium can grow at a temperature from about 35° C. to about 37° C. Further, the bacterium can grow on medium wherein the pH is from about 5.0 to about 7.5. In one aspect, the pH of the medium can be from about 6.0 to about 6.5. Media are currently known that are effective in promoting growth of the disclosed bacterium. Therefore, a person of skill would know which media would be effective in promoting the growth of the novel bacterium. Examples of media on which the bacterium can grow are shown below.

“Fermentation end-product” is used herein to include biofuels, chemicals, compounds suitable as liquid fuels, gaseous fuels, reagents, chemical feedstocks, chemical additives, processing aids, food additives, and other products. Examples of fermentation end-products include but are not limited to 1,4 diacids (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl)butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl)pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexane dione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexane diol, 4-methyl-1-(4-hydroxyphenyl)-2,3-hexane diol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexane diol, 4-methyl-1-(indole-3)-2,3-hexane diol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexane dione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, 1-decadecene, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid, formic acid, isoprenoids, and polyisoprenes, including rubber. Further, such products can include succinic acid, pyruvic acid, enzymes such as cellulases, polysaccharases, lipases, proteases, ligninases, and hemicellulases and may be present as a pure compound, a mixture, or an impure or diluted form.

The term “fatty acid comprising material” as used herein has its ordinary meaning as known to those skilled in the art and can comprise one or more chemical compounds that include one or more fatty acid moieties as well as derivatives of these compounds and materials that comprise one or more of these compounds. Common examples of compounds that include one or more fatty acid moieties include triacylglycerides, diacylglycerides, monoacylglycerides, phospholipids, lysophospholipids, free fatty acids, fatty acid salts, soaps, fatty acid comprising amides, esters of fatty acids and monohydric alcohols, esters of fatty acids and polyhydric alcohols including glycols (e.g. ethylene glycol, propylene glycol, etc.), esters of fatty acids and polyethylene glycol, esters of fatty acids and polyethers, esters of fatty acids and polyglycol, esters of fatty acids and saccharides, esters of fatty acids with other hydroxyl-containing compounds, etc. A fatty acid comprising material can be one or more of these compounds in an isolated or purified form. It can be a material that includes one or more of these compounds that is combined or blended with other similar or different materials. It can be a material where the fatty acid comprising material occurs with or is provided with other similar or different materials, such as vegetable and animal oils; mixtures of vegetable and animal oils; vegetable and animal oil byproducts; mixtures of vegetable and animal oil byproducts; vegetable and animal wax esters; mixtures, derivatives and byproducts of vegetable and animal wax esters; seeds; processed seeds; seed byproducts; nuts; processed nuts; nut byproducts; peels, animal matter; processed animal matter; byproducts of animal matter; corn; processed corn; corn byproducts; distiller's grains; beans; processed beans; bean byproducts; soy products; lipid containing plant, fish or animal matter; processed lipid containing plant or animal matter; byproducts of lipid containing plant, fish or animal matter; lipid containing microbial material; processed lipid containing microbial material; and byproducts of lipid containing microbial matter. Such materials can be utilized in liquid or solid forms. Solid forms include whole forms, such as cells, beans, and seeds; ground, chopped, slurried, extracted, flaked, milled, etc. The fatty acid portion of the fatty acid comprising compound can be a simple fatty acid, such as one that includes a carboxyl group attached to a substituted or un-substituted alkyl group. The substituted or unsubstituted alkyl group can be straight or branched, saturated or unsaturated. Substitutions on the alkyl group can include hydroxyls, phosphates, halogens, alkoxy, or aryl groups. The substituted or unsubstituted alkyl group can have 7 to 29 carbons and preferably 11 to 23 carbons (e.g., 8 to 30 carbons and preferably 12 to 24 carbons counting the carboxyl group) arranged in a linear chain with or without side chains and/or substitutions. Addition of the fatty acid comprising compound can be by way of adding a material comprising the fatty acid comprising compound.

The term “pH modifier” as used herein has its ordinary meaning as known to those skilled in the art and can include any material that will tend to increase, decrease or hold steady the pH of the broth or medium. A pH modifier can be an acid, a base, a buffer, or a material that reacts with other materials present to serve to raise, lower, or hold steady the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. In one embodiment, a buffer can be produced in the broth or medium or separately and used as an ingredient by at least partially reacting in acid or base with a base or an acid, respectively. When more than one pH modifiers are utilized, they can be added at the same time or at different times. In one embodiment, one or more acids and one or more bases is combined, resulting in a buffer. In one embodiment, media components, such as a carbon source or a nitrogen source serve as a pH modifier; suitable media components include those with high or low pH or those with buffering capacity. Exemplary media components include acid- or base-hydrolyzed plant polysaccharides having residual acid or base, ammonia fiber explosion (AFEX) treated plant material with residual ammonia, lactic acid, corn steep solids or liquor.

The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The microorganisms can be growing aerobically or anaerobically. They can be in any phase of growth, including lag (or conduction), exponential, transition, stationary, death, dormant, vegetative, sporulating, etc.

“Growth phase” is used herein to describe the type of cellular growth that occurs after the “Initiation phase” and before the “Stationary phase” and the “Death phase.” The growth phase is sometimes referred to as the exponential phase or log phase or logarithmic phase.

The term “plant polysaccharide” as used herein has its ordinary meaning as known to those skilled in the art and can comprise one or more polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “fermentable sugars” as used herein has its ordinary meaning as known to those skilled in the art and can include one or more sugars and/or sugar derivatives that can be utilized as a carbon source by the microorganism, including monomers, dimers, and polymers of these compounds including two or more of these compounds. In some cases, the organism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be utilized by the organism at hand. For some organisms, this would include conversion to monosaccharides, disaccharides, trisaccharides, and oligosaccharides of up to about seven monomer units, as well as similar sized chains of sugar derivatives and combinations of sugars and sugar derivatives. For some organisms, the allowable chain-length can be longer and for some organisms the allowable chain-length can be shorter.

The term “biomass” as used herein has its ordinary meaning as known to those skilled in the art and can include one or more biological materials that can be converted into a biofuel, chemical or other product. Biomass includes agricultural residues (corn stalks, grass, straw, grain hulls, bagasse, etc.), animal waste (manure from cattle, poultry, and hogs), Distillers Dried Solubles (DDS), Distillers Dried Grains (DDG), Condensed Distillers Solubles (CDS), Distillers Wet Grains (DWG), Distillers Dried Grains with Solubles (DDGS), woody materials (wood or bark, sawdust, timber slash, and mill scrap), municipal waste (waste paper, recycled toilet papers, yard clippings, etc.), and energy crops (poplars, willows, switch grass, alfalfa, prairie bluestem, algae, (including seaweed such as kelp or red macroalgae), and bacterial matter (e.g., bacterial cellulose).

One exemplary source of biomass is plant matter. Plant matter can be, for example, woody plant matter, non-woody plant matter, cellulosic material, lignocellulosic material, hemicellulosic material, carbohydrates, pectin, starch, inulin, fructans, glucans, corn, sugar cane, grasses, switchgrass, sorghum, high biomass sorghum, bamboo, algae and material derived from these. Plants can be in their natural state or genetically modified, e.g., to increase the cellulosic or hemicellulosic portion of the cell wall, or to produce additional exogenous or endogenous enzymes to increase the separation of cell wall components. Plant matter can be further described by reference to the chemical species present, such as proteins, polysaccharides and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corn steep solids, distillers grains, peels, husks, pits, fermentation waste, straw, lumber, sewage, garbage and food leftovers. Peels can be citrus which include, but are not limited to, tangerine peel, grapefruit peel, orange peel, tangerine peel, lime peel and lemon peel. These materials can come from farms, forestry, industrial sources, households, etc. Another non-limiting example of biomass is animal matter, including, for example milk, meat, fat, animal processing waste, and animal waste. Plant matter also includes maltose, corn syrup, Distillers Dried Solubles (DDS), Distillers Dried Grains (DDG), Condensed Distillers Solubles (CDS), Distillers Wet Grains (DWG), or Distillers Dried Grains with Solubles (DDGS). “Feedstock” is frequently used to refer to biomass being used for a process, such as those described herein. Plant matter comprises members of the kingdom Plantae, such as terrestrial plants and aquatic or marine plants. In one embodiment, terrestrial plants comprise crop plants (such as fruit, vegetable or grain plants). In one embodiment, aquatic or marine plants include, but are not limited to, sea grass, salt marsh grasses (such as Spartina sp. or Phragmites sp.) or the like. In one embodiment, a crop plant comprises a plant that is cultivated or harvested for oral consumption, or for utilization in an industrial, pharmaceutical, or commercial process. In one embodiment, crop plants include but are not limited to corn, wheat, rice, barley, soybeans, bamboo, cotton, crambe, jute, sorghum, high biomass sorghum, oats, tobacco, grasses, (e.g., Miscanthus grass or switch grass), trees (softwoods and hardwoods) or tree leaves, beans rape/canola, alfalfa, flax, sunflowers, safflowers, millet, rye, sugarcane, sugar beets, cocoa, tea, Brassica sp., cotton, coffee, sweet potatoes, flax, peanuts, clover; lettuce, tomatoes, cucurbits, cassaya, potatoes, carrots, radishes, peas, lentils, grapes, peppers, or pineapples; tree fruits or nuts such as citrus, apples, pears, peaches, apricots, walnuts, almonds, olives, avocadoes, bananas, or coconuts; flowers such as orchids, carnations and roses; nonvascular plants such as ferns; oil producing plants (such as castor bean, jatropha, or olives); or gymnosperms such as palms. Plant matter also comprises material derived from a member of the kingdom Plantae, such as woody plant matter, non-woody plant matter, cellulosic material, lignocellulosic material, or hemicellulosic material. Plant matter includes carbohydrates (such as pectin, starch, inulin, fructans, glucans, lignin, cellulose, or xylan). Plant matter also includes sugar alcohols, such as glycerol. In one embodiment, plant matter comprises a corn product, (e.g. corn stover, corn cobs, corn grain, corn steep liquor, corn steep solids, or corn grind), stillage, bagasse, leaves, pomace, or material derived therefrom. In another embodiment, plant matter comprises distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, pits, fermentation waste, skins, straw, seeds, shells, beancake, sawdust, wood flour, wood pulp, paper pulp, paper pulp waste streams, rice or oat hulls, bagasse, grass clippings, lumber, or food leftovers. These materials can come from farms, forestry, industrial sources, households, etc. In another embodiment, plant matter comprises an agricultural waste byproduct or side stream. In another embodiment, plant matter comprises a source of pectin such as citrus fruit (e.g., orange, grapefruit, lemon, or limes), potato, tomato, grape, mango, gooseberry, carrot, sugar beet, and apple, among others. In another embodiment, plant matter comprises plant peel (e.g., citrus peels) and/or pomace (e.g., grape pomace). In one embodiment, plant matter is characterized by the chemical species present, such as proteins, polysaccharides or oils. In one embodiment, plant matter is from a genetically modified plant. In one embodiment, a genetically-modified plant produces hydrolytic enzymes (such as a cellulase, hemicellulase, or pectinase etc.) at or near the end of its life cycles. In another embodiment, a genetically-modified plant encompasses a mutated species or a species that can initiate the breakdown of cell wall components. In another embodiment, plant matter is from a non-genetically modified plant.

Animal matter comprises material derived from a member of the kingdom Animaliae (e.g., bone meal, hair, heads, tails, beaks, eyes, feathers, entrails, skin, shells, scales, meat trimmings, hooves or feet) or animal excrement (e.g., manure). In one embodiment, animal matter comprises animal carcasses, milk, meat, fat, animal processing waste, or animal waste (manure from cattle, poultry, and hogs).

Biomass also comprises biological material derived from a member of the kingdoms Monera (e.g., Cyanobacteria) or Protista, e.g., algae (such as green algae, red algae, glaucophytes, Chrysophyta) or marine microflora, including plankton, or fungus-like members of Protista (such as slime molds, water molds, etc.).

Organic material comprises waste from farms, forestry, industrial sources, households or municipalities. In one embodiment, organic material comprises sewage, garbage, food waste (e.g., restaurant waste), waste paper, toilet paper, yard clippings, or cardboard.

The term “carbonaceous biomass” as used herein has its ordinary meaning as known to those skilled in the art and can include one or more biological materials that can be converted into a biofuel, chemical or other product. Carbonaceous biomass can comprise municipal waste (waste paper, recycled toilet papers, yard clippings, etc.), wood, plant material, plant matter, plant extract, bacterial matter (e.g. bacterial cellulose), distillers' grains, a natural or synthetic polymer, or a combination thereof.

In one embodiment, biomass does not include fossilized sources of carbon, such as hydrocarbons that are typically found within the top layer of the Earth's crust (e.g., natural gas, nonvolatile materials composed of almost pure carbon, like anthracite coal, etc.).

“Broth” is used herein to refer to inoculated medium at any stage of growth, including the point immediately after inoculation and the period after any or all cellular activity has ceased and can include the material after post-fermentation processing. It includes the entire contents of the combination of soluble and insoluble matter, suspended matter, cells and medium, as appropriate.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity is different from “titer” in that productivity includes a time term, and titer is analogous to concentration. Titer and Productivity can generally be measured at any time during the fermentation, such as at the beginning, the end, or at some intermediate time, with titer relating the amount of a particular material present or produced at the point in time of interest and the productivity relating the amount of a particular material produced per liter in a given amount of time. The amount of time used in the productivity determination can be from the beginning of the fermentation or from some other time, and go to the end of the fermentation, such as when no additional material is produced or when harvest occurs, or some other time as indicated by the context of the use of the term. “Overall productivity” refers to the productivity determined by utilizing the final titer and the overall fermentation time. “Productivity to maximum titer” refers to the productivity determined utilizing the maximum titer and the time to achieve the maximum titer. “Instantaneous productivity” refers to the productivity at a moment in time and can be determined from the slope of the titer v. time curve for the compound of interest, or by other appropriate means as determined by the circumstances of the operation and the context of the language. “Incremental productivity” refers to productivity over a portion of the fermentation time, such as several minutes, an hour, or several hours. Frequently, an incremental productivity is used to imply or approximate instantaneous productivity. Other types of productivity can be used as well, with the context indicating how the value should be determined.

“Titer” refers to the amount of a particular material present in a fermentation broth. It is similar to concentration and can refer to the amount of material made by the organism in the broth from all fermentation cycles, or the amount of material made in the current fermentation cycle or over a given period of time, or the amount of material present from whatever source, such as produced by the organism or added to the broth. Frequently, the titer of soluble species will be referenced to the liquid portion of the broth, with insolubles removed, and the titer of insoluble species will be referenced to the total amount of broth with insoluble species being present, however, the titer of soluble species can be referenced to the total broth volume and the titer of insoluble species can be referenced to the liquid portion, with the context indicating the which system is used with both reference systems intended in some cases. Frequently, the value determined referenced to one system will be the same or a sufficient approximation of the value referenced to the other. “Concentration” when referring to material in the broth generally refers to the amount of a material present from all sources, whether made by the organism or added to the broth. Concentration can refer to soluble species or insoluble species, and is referenced to either the liquid portion of the broth or the total volume of the broth, as for “titer.”

The term “biocatalyst” as used herein has its ordinary meaning as known to those skilled in the art and can include one or more enzymes and microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2CO₂

and the theoretical maximum conversion efficiency, or yield, is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art.

“Pretreatment” or “pretreated” is used herein to refer to any mechanical, chemical, thermal, biochemical process or combination of these processes whether in a combined step or performed sequentially, that achieves disruption or expansion of the biomass so as to render the biomass more susceptible to attack by enzymes and/or microorganisms. In one embodiment, pretreatment includes removal or disruption of lignin so as to make the cellulose and hemicellulose polymers in the plant biomass more available to cellulolytic enzymes and/or microorganisms, for example, by treatment with acid or base. In one embodiment, pretreatment includes the use of a microorganism of one type to render plant polysaccharides more accessible to microorganisms of another type, for example, by treatment with acid or base. In one embodiment, pretreatment includes disruption or expansion of cellulosic and/or hemicellulosic material. Steam explosion, and ammonia fiber expansion (or explosion) (AFEX) are well known thermal/chemical techniques. Hydrolysis, including methods that utilize acids, bases, and/or enzymes can be used. Other thermal, chemical, biochemical, enzymatic techniques can also be used.

“Fed-batch” or “fed-batch fermentation” is used herein to include methods of culturing microorganisms where nutrients, other medium components, or biocatalysts (including, for example, enzymes, fresh organisms, extracellular broth, genetically modified plants and/or organisms, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. During a fed-batch fermentation, the broth volume can increase, at least for a period, by adding medium or nutrients to the broth while fermentation organisms are present. In some fed-batch fermentations, the broth volume can be insensitive to the addition of nutrients and in some cases not change from the addition of nutrients. Suitable nutrients which can be utilized include those that are soluble, insoluble, and partially soluble, including gasses, liquids and solids. In one embodiment, a fed-batch process is referred to with a phrase such as, “fed-batch with cell augmentation.” This phrase can include an operation where nutrients and cells are added or one where cells with no substantial amount of nutrients are added. The more general phrase “fed-batch” encompasses these operations as well. The context where any of these phrases is used will indicate to one of skill in the art the techniques being considered.

A term “phytate” as used herein has its ordinary meaning as known to those skilled in the art can be include phytic acid, its salts, and its combined forms as well as combinations of these.

“Sugar compounds” is used herein to include monosaccharide sugars, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length.

“Dry cell weight” is used herein to refer to a method of determining the cell content of a broth or inoculum, and the value so determined. The method can include rinsing or washing a volume of broth followed by drying and weighing the residue. In some cases, a sample of broth is simply centrifuged with the layer containing cells collected, dried, and weighed. Frequently, the broth is centrifuged, then resuspended in water or a mixture of water and other ingredients, such as a buffer, ingredients to create an isotonic condition, ingredients to control any change in osmotic pressure, etc. The centrifuge-resuspend steps can be repeated, if desired, and different resuspending solutions can be used prior to the final centrifuging and drying. When an insoluble medium component is present, the presence of the insoluble component can be ignored, with the value determined as above. Methods when insoluble medium components are present include those where the insoluble component is reacted to a soluble form, dissolved or extracted into a different solvent that can include water, or separated by an appropriate method, such as by centrifugation, gradient centrifugation, flotation, filtration, or other suitable technique or combination of techniques.

Clostridium phytofermentans Q.17, Q.18, Q.19, and Q.20

Biocatalysts Clostridium phytofermentans strains Q.17, Q.18, Q.19 and Q.20 are fast-growing, high yielding strains of Clostridium phytofermentans that do not demonstrate repression of cellulose hydrolysis in the presence of glucose or other sugars, and can, in some embodiments, be defined based on the phenotypic and genotypic characteristics of the cultured strain as described infra. Aspects described herein generally include systems, methods, and compositions for producing fuels, such as ethanol, and/or other useful organic products involving, for example, Q.17, Q.18, Q.19 or Q.20, and/or any other strain of the species, including those which can be derived from these strains, including genetically modified strains, or strains separately isolated. Some exemplary species can be defined using standard taxonomic considerations (Stackebrandt and Goebel, International Journal of Systematic Bacteriology, 44:846-9, 1994): Strains with 16S rRNA sequence homology values of 98% and higher as compared to the type Q.17, Q.18, Q.19 or Q.20, and strains with DNA re-association values of at least about 70% can be considered Q.17, Q.18, Q.19 or Q.20. For example, strains with 16S rRNA sequence homology values of at least 97.1, 97.2, 97.3, 97.4, 97.5, 97.6, 97.7, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9% can be considered Q.17, Q.18, Q.19 or Q.20. In one embodiment, strains with DNA re-association values of at least about 71, 72, 73, 74, 75, 76, 77, 78, 79, 80, 81, 82, 83, 84, 85, 86, 87, 88, 89, 90, 91, 92, 93, 94, 95, 96, 97, 98, 99% can be considered Q.17, Q.18, Q.19 or Q.20. Considerable evidence exists to indicate that many microorganisms which have 70% or greater DNA re-association values also have at least 98% DNA sequence identity and share phenotypic traits defining a species. Analyses of the genome sequence of Q.17, Q.18, Q.19 or Q.20 indicate the presence of large numbers of genes and genetic loci that are likely to be involved in mechanisms and pathways for plant polysaccharide fermentation, giving rise to the unusual fermentation properties of these biocatalysts which can be found in all or nearly all strains of the species Q.17, Q.18, Q.19 or Q.20 and can be natural isolates, or genetically modified strains.

### Attributes of Q.17, Q.18, Q.19 or Q.20

In one embodiment, the microorganisms, Q.17, Q.18, Q.19 or Q.20, provide useful advantages for the conversion of biomass to ethanol and other products. One advantage of this microorganism is its ability to produce enzymes capable of hydrolyzing polysaccharides and higher molecular weight saccharides to lower molecular weight saccharides, such as oligosaccharides, disaccharides, and monosaccharides. In one embodiment, Q.17, Q.18, Q.19 or Q.20 produce a hydrolytic cellulase enzyme which facilitates fermenting of a biomass material and is not repressed by glucose or other monomeric sugars, or other regulatory factors, such as the hydrolysis of hemicellulose. Examples of biomass material that can be fermented include, but are not limited to, cellulosic, hemicellulosic, lignocellulosic materials; pectins; starches; wood; paper; agricultural products; forest waste; tree waste; tree bark; leaves; grasses; sawgrass; woody plant matter; non-woody plant matter; nonvascular plants, carbohydrates; pectin; starch; inulin; fructans; glucans; corn; sugarcane; grasses; bamboo, algae, and material derived from these materials. The organisms can usually produce these enzymes as needed, frequently without excessive production of unnecessary hydrolytic enzymes, or in one embodiment, one or more enzymes is added to further improve the organism's production capability. This ability to produce a very wide range of deregulated hydrolytic enzymes can give Q.17, Q.18, Q.19 or Q.20 and the associated technology distinct advantages in biomass fermentation, especially those fermentations not utilizing simple sugars as the feedstock. Various fermentation conditions can enhance the activities of the organism, resulting in higher yields, higher rates of saccharification, higher productivity, greater product selectivity, and/or greater conversion efficiency. In one embodiment, fermentation conditions include fed batch operation and fed batch operation with cell augmentation; addition of complex nitrogen sources such as corn steep powder or yeast extract; addition of specific amino acids including proline, glycine, isoleucine, and/or histidine; addition of a complex material containing one or more of these amino acids; addition of other nutrients or other compounds such as phytate, proteases enzymes, or polysaccharase enzymes. In one embodiment, fermentation conditions can include supplementation of a medium with an organic nitrogen source. In another embodiment, fermentation conditions can include supplementation of a medium with an inorganic nitrogen source. In one embodiment, the addition of one material provides supplements that fit into more than one category, such as providing amino acids and phytate.

In one embodiment, the Q.17, Q.18, Q.19 or Q.20 organism is used to hydrolyze various higher saccharides (higher molecular weight) present in biomass to lower saccharides (lower molecular weight), such as in preparation for fermentation to produce ethanol, hydrogen, or other chemicals such as organic acids including formic acid, acetic acid, and lactic acid. Another advantage of Q.17, Q.18, Q.19 or Q.20 is its ability to hydrolyze polysaccharides and higher saccharides that contain hexose sugar units or that contain pentose sugar units, and that contain both, into lower saccharides and in some cases monosaccharides. These enzymes and/or the hydrolysate can be used in fermentations to produce various products including fuels, and other chemicals. Another advantage of Q.17, Q.18, Q.19 or Q.20 is its ability to produce ethanol, hydrogen, and other fuels or compounds such as organic acids including acetic acid, formic acid, glutamic acid, aspartic acid, malic acid, and lactic acid from lower sugars (lower molecular weight) such as monosaccharides. Another advantage of Q.17, Q.18, Q.19 or Q.20 is its ability to perform the combined steps of hydrolyzing a higher molecular weight biomass containing sugars and/or higher saccharides or polysaccharides to lower sugars and fermenting these lower sugars into desirable products including ethanol, hydrogen, and other chemicals and compounds such as organic acids including glutamic acid, aspartic acid, malic acid, formic acid, acetic acid, and lactic acid.

Another advantage of Q.17, Q.18, Q.19 or Q.20 is its ability to grow under conditions that include elevated ethanol concentration, high sugar concentration, low sugar concentration, utilize insoluble carbon sources, and/or operate under anaerobic conditions. These characteristics, in various combinations, can be used to achieve operation with long fermentation cycles and can be used in combination with batch fermentations, fed batch fermentations, self-seeding/partial harvest fermentations, and recycle of cells from the final fermentation as inoculum.

A further advantage of Q.17, Q.18, Q.19 or Q.20 is the ability to use any of these strains in an SSF or SHF process without the added cost of exogenous enzymes. The hydrolysis of cellulose and other polymers is a rate-limiting step in the production of ethanol and other products by biocatalysts. Native cellulases of organisms such as Clostridium phytofermentans are more efficient at hydrolysis and transport of smaller carbonaceous polymers and their conversion into metabolic products. With higher hydrolysis rates and little or no feedback repression, the rate of product formation is increased.

In one example, the process for converting biomass material into ethanol includes pretreating the biomass material (e.g., “feedstock”), hydrolyzing the pretreated biomass to convert polysaccharides to oligosaccharides, further hydrolyzing the oligosaccharides to monosaccharides, and converting the monosaccharides to ethanol. In one example, the biomass can be hydrolyzed directly to monosaccharides or other saccharides that are utilized by the fermentation organism to produce ethanol or other products. If a different final product is desired, such as hydrocarbons, hydrogen, methane, hydroxy compounds such as alcohols (e.g. butanol, propanol, methanol, etc.), carbonyl compounds such as aldehydes and ketones (e.g. acetone, formaldehyde, 1-propanal, etc.), organic acids, derivatives of organic acids such as esters (e.g. wax esters, glycerides, etc.) and other functional compounds including, but not limited to, 1,2-propanediol, 1, 3-propanediol, lactic acid, formic acid, acetic acid, malic acid, glutamic acid, succinic acid, pyruvic acid, enzymes such as cellulases, polysaccharases, lipases, proteases, ligninases, and hemicellulases, the monosaccharides can be used in the biosynthesis of that particular compound. Biomass material that can be utilized includes woody plant matter, non-woody plant matter, cellulosic material, lignocellulosic material, hemicellulosic material, carbohydrates, pectin, starch, inulin, fructans, glucans, corn, algae, sugarcane, grasses, switchgrass, bamboo, citrus peels, sorghum, high biomass sorghum, oat hulls, and material derived from these. The final product can then be separated and/or purified, as indicated by the properties for the desired final product. In some instances, compounds related to sugars such as sugar alcohols or sugar acids can be utilized as well.

In one embodiment, more than one of these steps can occur at any given time. For example, hydrolysis of the pretreated feedstock and hydrolysis of the oligosaccharides can occur simultaneously, and one or more of these can occur simultaneously to the conversion of monosaccharides to ethanol.

In another embodiment, an enzyme can directly convert the polysaccharide to monosaccharides. In some instances, an enzyme can hydrolyze the polysaccharide to oligosaccharides and the enzyme or another enzyme can hydrolyze the oligosaccharides to monosaccharides.

In another embodiment, the enzymes present in the fermentation can be produced separately and then added to the fermentation or they can be produced by microorganisms present in the fermentation. In one embodiment, the microorganisms present in the fermentation produces some enzymes. In another embodiment, enzymes are produced separately and added to the fermentation.

The overall conversion of pretreated biomass to final product can occur at high rates. High rates of conversion can be achieved if enzymes for each conversion step are present with sufficiently high activity. If one of these enzymes is missing or is present in insufficient quantities, the production rate of ethanol, or other desired product can be reduced. The production rate can also be reduced if the microorganisms responsible for the conversion of monosaccharides to product only slowly take up monosaccharides and/or have only limited capability for translocation of the monosaccharides and intermediates produced during the conversion to ethanol.

In another embodiment, the enzymes of the method are produced by Q.17, Q.18, Q.19 or Q.20, including a range of hydrolytic enzymes suitable for the biomass materials used in the fermentation methods. In one embodiment, Q.17, Q.18, Q.19 or Q.20 is grown under conditions appropriate to induce and/or promote production of the enzymes needed for the saccharification of the polysaccharide present. The production of these enzymes can occur in a separate vessel, such as a seed fermentation vessel or other fermentation vessel, or in the production fermentation vessel where ethanol production occurs. When the enzymes are produced in a separate vessel, they can, for example, be transferred to the production fermentation vessel along with the cells, or as a relatively cell free solution liquid containing the intercellular medium with the enzymes. When the enzymes are produced in a separate vessel, they can also be dried and/or purified prior to adding them to the production fermentation vessel. The conditions appropriate for production of the enzymes are frequently managed by growing the cells in a medium that includes the biomass that the cells will be expected to hydrolyze in subsequent fermentation steps. Additional medium components, such as salt supplements, growth factors, and cofactors including, but not limited to phytate, amino acids, and peptides can also assist in the production of the enzymes utilized by the microorganism in the production of the desired products.

### Feedstock and Pretreatment of Feedstock

In one embodiment, the feedstock contains cellulosic, hemicellulosic, and/or lignocellulosic material. The feedstock can be derived from agricultural crops, crop residues, trees, woodchips, sawdust, paper, cardboard, grasses, algae and other biomass sources.

Cellulose is a linear polymer of glucose where the glucose units are connected vial β(1→4) linkages. Hemicellulose is a branched polymer of a number of sugar monomers including glucose, xylose, mannose, galactose, rhamnose and arabinose, and can have sugar acids such as mannuronic acid and galacturonic acid present as well. Lignin is a cross-linked, racemic macromolecule of mostly p-coumaryl alcohol, conferyl alcohol and sinapyl alcohol. These three polymers occur together in lignocellusic materials in plant biomass. The different characteristics of the three polymers can make hydrolysis of the combination difficult as each polymer tends to shield the others from enzymatic attack.

In one embodiment, methods are provided for the pretreatment of feedstock used in the fermentation and production of the biofuels and ethanol. The pretreatment steps can include mechanical, thermal, pressure, chemical, thermochemical, and/or biochemical tests pretreatment prior to being used in a bioprocess for the production of fuels and chemicals, but untreated biomass material can be used in the process as well. Mechanical processes can reduce the particle size of the biomass material so that it can be more conveniently handled in the bioprocess and can increase the surface area of the feedstock to facilitate contact with chemicals/biochemicals/biocatalysts. Mechanical processes can also separate one type of biomass material from another. The biomass material can also be subjected to thermal and/or chemical pretreatments to render plant polymers more accessible. Multiple steps of treatment can also be used.

Mechanical processes include, are not limited to, washing, soaking, milling, size reduction, screening, shearing, size classification and density classification processes. Chemical processes include, but are not limited to, bleaching, oxidation, reduction, acid treatment, base treatment, sulfite treatment, acid sulfite treatment, basic sulfite treatment, ammonia treatment, and hydrolysis. Thermal processes include, but are not limited to, sterilization, ammonia fiber expansion or explosion (“AFEX”), steam explosion, holding at elevated temperatures, pressurized or unpressurized, in the presence or absence of water, and freezing. Biochemical processes include, but are not limited to, treatment with enzymes, including enzymes produced by genetically-modified plants, and treatment with microorganisms. Various enzymes that can be utilized include cellulase, amylase, β-glucosidase, xylanase, gluconase, and other polysaccharases; lysozyme; laccase, and other lignin-modifying enzymes; lipoxygenase, peroxidase, and other oxidative enzymes; proteases; and lipases. One or more of the mechanical, chemical, thermal, thermochemical, and biochemical processes can be combined or used separately. Such combined processes can also include those used in the production of paper, cellulose products, microcrystalline cellulose, and cellulosics and can include pulping, kraft pulping, acidic sulfite processing. The feedstock can be a side stream or waste stream from a facility that utilizes one or more of these processes on a biomass material, such as cellulosic, hemicellulosic or lignocellulosic material. Examples include paper plants, cellulosics plants, cotton processing plants, and microcrystalline cellulose plants. The feedstock can also include cellulose-containing or cellulosic containing waste materials. The feedstock can also be biomass materials, such as wood, grasses, corn, starch, or sugar, produced or harvested as an intended feedstock for production of ethanol or other products such as by Q.17, Q.18, Q.19 or Q.20 biocatalysts.

In another embodiment, a method can utilize a pretreatment process disclosed in U.S. Patents and Patent Applications US20040152881, US20040171136, US20040168960, US20080121359, US20060069244, US20060188980, US20080176301, U.S. Pat. Nos. 5,693,296, 6,262,313, US20060024801, U.S. Pat. Nos. 5,969,189, 6,043,392, US20020038058, U.S. Pat. No. 5,865,898, U.S. Pat. No. 5,865,898, U.S. Pat. No. 6,478,965, 5,986,133, or US20080280338, each of which is incorporated by reference herein in its entirety

In another embodiment, the AFEX process is be used for pretreatment of biomass. In one embodiment, the AFEX process is used in the preparation of cellulosic, hemicellulosic or lignocellulosic materials for fermentation to ethanol or other products. The process generally includes combining the feedstock with ammonia, heating under pressure, and suddenly releasing the pressure. Water can be present in various amounts. The AFEX process has been the subject of numerous patents and publications.

In another embodiment, the pretreatment of biomass comprises the addition of calcium hydroxide to a biomass to render the biomass susceptible to degradation. Pretreatment comprises the addition of calcium hydroxide and water to the biomass to form a mixture, and maintaining the mixture at a relatively high temperature. Alternatively, an oxidizing agent, selected from the group consisting of oxygen and oxygen-containing gasses, can be added under pressure to the mixture. Examples of carbon hydroxide treatments are disclosed in U.S. Pat. No. 5,865,898 to Holtzapple and S. Kim and M. T. Holzapple, Bioresource Technology, 96, (2005) 1994, incorporated by reference herein in its entirety.

In one embodiment, pretreatment of biomass comprises dilute acid hydrolysis using, e.g., acetic acid, oxalic acid, malic acid, carboxylic acid, lactic acid, citric acid and the like. Examples of dilute acid hydrolysis treatment are disclosed in T. A. Lloyd and C. E Wyman, Bioresource Technology, (2005) 96, 1967), incorporated by reference herein in its entirety.

In another embodiment, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety.

In one embodiment, pretreatment of biomass comprises aqueous ammonia recycle process (ARP). Examples of aqueous ammonia recycle process are described in T. H. Kim and Y. Y. Lee, Bioresource Technology, (2005)₉₆, 2007, incorporated by reference herein in its entirety.

In one embodiment, the above mentioned methods have two steps: a pretreatment step that leads to a wash stream, and an enzymatic hydrolysis step of pretreated-biomass that produces a hydrolysate stream. In the above methods, the pH at which the pretreatment step is carried out includes acid hydrolysis, hot water pretreatment, steam explosion or alkaline reagent based methods (AFEX, ARP, and lime pretreatments). Dilute acid and hot water treatment methods solubilize mostly hemicellulose, whereas methods employing alkaline reagents remove most lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual biomass leads to mixed sugars (C5 and C6) in the alkali based pretreatment methods, while glucose is the major product in the hydrolyzate from the low and neutral pH methods. In one embodiment, the treated material is additionally treated with catalase or another similar chemical, chelating agents, surfactants, and other compounds to remove or bind impurities or toxic chemicals or further release polysaccharides.

In one embodiment, pretreatment of biomass comprises ionic liquid pretreatment. Biomass can be pretreated by incubation with an ionic liquid (IL), followed by IL extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

In another embodiment, a method can utilize a pretreatment process disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., et al., U.S. Pat. No. 5,939,544 to Karstens, et al., U.S. Pat. No. 5,473,061 to Bredereck, et al., U.S. Pat. No. 6,416,621 to Karstens., U.S. Pat. No. 6,106,888 to Dale, et al., U.S. Pat. No. 6,176,176 to Dale, et al., PCT publication WO2008/020901 to Dale, et al., Felix, A., et al., Anim Prod. 51, 47-61 (1990), Wais, A. C., Jr., et al., Journal of Animal Science, 35, No. 1, 109-112 (1972), which are incorporated herein by reference in their entireties.

Alteration of the pH of a pretreated feedstock can be accomplished by washing the feedstock (e.g., with water) one or more times to remove an alkaline or acidic substance, or other substance used or produced during pretreatment. Washing can comprise exposing the pretreated feedstock to an equal volume of water 2, 3, 4, 5, 6, 7, 8, 9, 10, 11, 12, 13, 14, 15, 16, 17, 18, 19, 20, 21, 22, 23, 24, 25 or more times. In another embodiment, a pH modifier can be added. For example, an acid, a buffer, or a material that reacts with other materials present can be added to modulate the pH of the feedstock. In one embodiment, more than one pH modifier can be used, such as one or more bases, one or more bases with one or more buffers, one or more acids, one or more acids with one or more buffers, or one or more buffers. When more than one pH modifiers are utilized, they can be added at the same time or at different times. Other non-limiting exemplary methods for neutralizing feedstocks treated with alkaline substances have been described, for example in U.S. Pat. Nos. 4,048,341; 4,182,780; and 5,693,296.

In one embodiment, one or more acids can be combined, resulting in a buffer. Suitable acids and buffers that can be used as pH modifiers include any liquid or gaseous acid that is compatible with the microorganism. Non-limiting examples include peroxyacetic acid, sulfuric acid, lactic acid, citric acid, phosphoric acid, and hydrochloric acid. In some instances, the pH can be lowered to neutral pH or acidic pH, for example a pH of 7.0, 6.5, 6.0, 5.5, 5.0, 4.5, 4.0, or lower. In some embodiments, the pH is lowered and/or maintained within a range of about pH 4.5 to about 7.1, or about 4.5 to about 6.9, or about pH 5.0 to about 6.3, or about pH 5.5 to about 6.3, or about pH 6.0 to about 6.5, or about pH 5.5 to about 6.9 or about pH 6.2 to about 6.7.

In another embodiment, biomass can be pretreated at an elevated temperature and/or pressure. In one embodiment, biomass is pretreated at a temperature range of 20° C. to 400° C. In another embodiment, biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment, steam can be injected into a biomass containing vessel. In another embodiment, the steam, hot water, or hot gas can be injected into a vessel jacket such that it heats, but does not directly contact the biomass.

In another embodiment, a biomass can be treated at an elevated pressure. In one embodiment, biomass is pre treated at a pressure range of about 1 psi to about 30 psi. In another embodiment, biomass is pre treated at a pressure or about 1 psi, 2 psi, 3 psi, 4 psi, 5 psi, 6 psi, 7 psi, 8 psi, 9 psi, 10 psi, 12 psi, 15 psi, 18 psi, 20 psi, 22 psi, 24 psi, 26 psi, 28 psi, 30 psi or more. In some embodiments, biomass can be treated with elevated pressures by the injection of steam into a biomass containing vessel. In one embodiment, the biomass can be treated to vacuum conditions prior or subsequent to alkaline or acid treatment or any other treatment methods provided herein.

In one embodiment, alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In one embodiment, of the present disclosure, the pretreatment step includes a step of solids recovery. The solids recovery step can be during or after pretreatment (e.g., acid or alkali pretreatment), or before the drying step. In one embodiment, the solids recovery step provided by the methods of the present disclosure includes the use of a sieve, filter, screen, or a membrane for separating the liquid and solids fractions. In one embodiment, a suitable sieve pore diameter size ranges from about 0.001 microns to 8 mm, such as about 0.005 microns to 3 mm or about 0.01 microns to 1 mm. In one embodiment, a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more.

In one embodiment, biomass (e.g. corn stover or bagasse) is processed or pretreated prior to fermentation. In one embodiment, a method of pre-treatment includes but is not limited to, biomass particle size reduction, such as for example shredding, milling, chipping, crushing, grinding, or pulverizing. In one embodiment, biomass particle size reduction can include size separation methods such as sieving, or other suitable methods known in the art to separate materials based on size. In one embodiment, size separation can provide for enhanced yields. In one embodiment, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles can allow the recycling of the larger particles back into the size reduction process, thereby increasing the final yield of processed biomass. In one embodiment, a fermentative mixture is provided which comprises a pretreated lignocellulosic feedstock comprising less than about 50% of a lignin component present in the feedstock prior to pretreatment and comprising more than about 60% of a hemicellulose component present in the feedstock prior to pretreatment; and a microorganism capable of fermenting a five-carbon sugar, such as xylose, arabinose or a combination thereof, and a six-carbon sugar, such as glucose, galactose, mannose or a combination thereof. In some instances, pretreatment of the lignocellulosic feedstock comprises adding an alkaline substance which raises the pH to an alkaline level, for example NaOH. In one embodiment, NaOH is added at a concentration of about 0.5% to about 2% by weight of the feedstock. In one embodiment, pretreatment also comprises addition of a chelating agent. In one embodiment, the microorganism is a bacterium, such as a member of the genus Clostridium, for example Clostridium phytofermentans, including Q.17, Q.18, Q.19 or Q.20.

The present disclosure also provides a fermentative mixture comprising: a cellulosic feedstock pre-treated with an alkaline substance which maintains an alkaline pH, and at a temperature of from about 80° C. to about 120° C.; and a microorganism capable of fermenting a five-carbon sugar and a six-carbon sugar. In one embodiment, the five-carbon sugar is xylose, arabinose, or a combination thereof. In one embodiment, the six-carbon sugar is glucose, galactose, mannose, or a combination thereof. In one embodiment, the alkaline substance is NaOH. In some embodiments, NaOH is added at a concentration of about 0.5% to about 2% by weight of the feedstock. In one embodiment, the microorganism is a bacterium, such as a member of the genus Clostridium, for example Clostridium phytofermentans, Q.17, Q.18, Q.19 or Q.20. In still another embodiment, the microorganism is genetically modified to enhance activity of one or more hydrolytic enzymes.

Further provided herein is a fermentative mixture comprising a cellulosic feedstock pre-treated with an alkaline substance which increases the pH to an alkaline level, at a temperature of from about 80° C. to about 120° C.; and a microorganism capable of uptake and fermentation of an oligosaccharide. In one embodiment, the alkaline substance is NaOH. In some embodiments, NaOH is added at a concentration of about 0.5% to about 2% by weight of the feedstock. In one embodiment, the microorganism is a bacterium, such as a member of the genus Clostridium, for example Clostridium phytofermentans, including Q.17, Q.18, Q.19 or Q.20. In one embodiment, the microorganism is genetically modified to express or increase expression of an enzyme capable of hydrolyzing said oligosaccharide, a transporter capable of transporting the oligosaccharide, or a combination thereof.

In one embodiment, pretreatment of biomass comprises enzyme hydrolysis. In one embodiment, a biomass is pretreated with an enzyme or a mixture of enzymes, e.g., endonucleases, exonucleases, cellobiohydrolases, cellulase, beta-glucosidases, glycoside hydrolases, glycosyltransferases, lyases, esterases and proteins containing carbohydrate-binding modules. In one embodiment, the enzyme or mixture of enzymes is one or more individual enzymes with distinct activities. In another embodiment, the enzyme or mixture of enzymes can be enzyme domains with a particular catalytic activity. For example, an enzyme with multiple activities can have multiple enzyme domains, including, for example, glycoside hydrolases, glycosyltransferases, lyases and/or esterases catalytic domains.

In one embodiment, pretreatment of biomass comprises enzyme hydrolysis with one or more enzymes from a Q.17, Q.18, Q.19 or Q.20 biocatalyst. In one embodiment, pretreatment of biomass comprises enzyme hydrolysis with one or more enzymes from Q.17, Q.18, Q.19 or Q.20, wherein the one or more enzyme is selected from the group consisting of endonucleases, exonucleases, cellobiohydrolases, beta-glucosidases, glycoside hydrolases, glycosyltransferases, lyases, esterases and proteins containing carbohydrate-binding modules. In one embodiment, biomass can be pretreated with a hydrolase identified in C. phytofermentans.

In one embodiment, pretreatment of biomass comprises enzyme hydrolysis with one or more of enzymes listed in Table 1. Table 1 show examples of known activities of some of the glycoside hydrolases, lyases, esterases, and proteins containing carbohydrate-binding modules family members predicted to be present in Clostridia, for example, C. phytofermentans. Known activities are listed by activity and corresponding PC number as determined by the International Union of Biochemistry and Molecular Biology.

TABLE 1
Known activities of glycoside hydrolase family members

| Glycoside | | Number of domains |
|---|---|---|
| Hydrolase | | predicted in |
| Family | Known activities | C. phytofermentans |
| 1 | beta-glucosidase (EC 3.2.1.21); beta-galactosidase (EC 3.2.1.23); beta- | 1 |
| | mannosidase (EC 3.2.1.25); beta-glucuronidase (EC 3.2.1.31); beta-D- | |
| | fucosidase (EC 3.2.1.38); phlorizin hydrolase (EC 3.2.1.62); 6- | |
| | phospho--galactosidase (EC 3.2.1.85); 6-phospho-beta-glucosidase | |
| | (EC 3.2.1.86); strictosidinebeta-glucosidase (EC 3.2.1.105); lactase (EC | |
| | 3.2.1.108); amygdalinbeta-glucosidase (EC 1 3.2.1.117); prunasin beta- | |
| | glucosidase (EC 3.2.1.118); raucaifricine beta-glucosidase (EC | |
| | 3.2.1.125); thioglucosidase (EC 3.2.1.147); beta-primeverosidase (EC | |
| | 3.2.1.149); isoflavonod 7-0-beta-apiosyl--glucosidase (EC 3.2.1.161); | |
| | hydroxyisourate hydrolase (EC_3.—.—.—);_beta-glycosidase_(EC_3.2.1.—) | |
| 2 | beta-galactosidase (EC 3.2.1.23); beta-mannosidase (EC 3.2.1.25); | 5 |
| | beta-glucuronidase (EC 3.2.1.31); mannosylglycoprotein 5 endo-beta- | |
| | mannosidase (EC 3.2.1.152); exo-beta glucosaminidase_(E 3.2.1.—) | |
| 3 | beta-glucosidase (EC 3.2.1.21); xylan 1,4-beta-xylosidase (EC | 8 |
| | 3.2.1.37); beta-N-acetylhexosaminidase (EC 3.2.1.52); glucan 1,3- | |
| | beta-glucosiclase (EC 3.2.1.58); glucan 1,4-beta-glucosidase (EC 3.2.1.74); | |
| | exo-1,3-1,4-glucanase (EC 3.2.1.—); alpha-L arabinofuranosidase | |
| | (EC 3.2.1.55). | |
| 4 | maltose-6-phosphate glucosidase (EC 3.2.1.122); alpha glucosidase | 3 |
| | (EC 3.2.1.20); alpha-galactosidase (EC 3.2.1.22); 6-phospho-beta- | |
| | glucosidase (EC 3.2.1.86); alpha-glucuronidase (EC 3.2.1.139). | |
| 5 | chitosanase (EC 3.2.1.132); beta-mannosidase (EC 3.2.1.25); Cellulase | 3 |
| | (EC 3.2.1.4); glucan 1,3-beta-glucosidase (EC 3.2.1.58); licheninase | |
| | (EC 3.2.1.73); glucan endo-1,6-beta-glucosidase (EC 3.2.1.75); mannan | |
| | endo-1,4-beta-mannosidase (EC 3.2.1.78); 3 Endo-1,4-beta-xylanase | |
| | (EC 3.2.1.8); cellulose 1,4-beta-cellobiosidase (EC 3.2.1.91); endo-1,6- | |
| | beta-galactanase (EC 3.2.1.—); beta-1,3-mannanase (EC 3.2.1.—); | |
| | xyloglucan-specific endo-beta-1,4-glucanase (EC 3.2.1.151) | |
| 8 | chitosanase (EC 3.2.1.132); cellulase (EC 3.2.1.4); licheninase (EC | 1 |
| | 3.2.1.73); endo-1,4-beta-xylanase (EC 3.2.1.8); reducing-end-xylose | |
| | releasing exo-oligoxylanase (EC 3.2.1.156) | |
| 9 | endoglucanase (EC 3.2.1.4); cellobiohydrolase (EC 3.2.1.91); beta- | 1 |
| | glucosidase (EC 3.2.1.21) | |
| 10 | xylanase (EC 3.2.1.8); endo-1,3-beta-xylanase (EC 3.2.1.32) | 6 |
| 11 | xylanase (EC 3.2.1.8). | 1 |
| 12 | endoglucanase (EC 3.2.1.4); xyloglucan hydrolase (EC 3.2.1.151); | 1 |
| | beta-1,3-1,4-glucanase (EC 3.2.1.73); xyloglucan endotransglycosylase | |
| | (EC 2.4.1.207) | |
| 13 | apha-amylase (EC 3.2.1.1); pullulanase (EC 3.2.1.41); | 7 |
| | cyclomaltodextrin glucanotransferase (EC 2.4.1.19); | |
| | cyclornaltodextrinase (EC 3.2.1.54); trehalose-6-phosphate hydrolase | |
| | (EC 3.2.1.93); oligo-alpha-glucosiclase (EC 3.2.1.10); maltogenic | |
| | amylase (EC 3.2.1.133); neopullulanase (EC 3.2.1.135); alpha- | |
| | glucosidase (EC 3.2.1.20); maltotetraose-forming 3 alpha-amylase (EC | |
| | 3.2.1.60); isoamylase (EC 3.2.1.68); glucodextranase (EC 12.170); | |
| | maltohexaose-forming alphaamylase (EC 3.2.1.98); branching enzyme | |
| | (EC 2.4.1.18); trehalose synthase (EC 5.4.99.16); 4--glucanotransferase | |
| | (EC 2.4.1.25); maltopentaose-forming-amylase (EC 3.2.1.—); | |
| | amylosucrase (EC 2.4.1.4): sucrose phosphorylase (EC 2.4.1.7); malto- | |
| | oligosyltrehalose trehalohydrolase (EC 3.2.1.141); isomaltulose | |
| | synthase (EC 5.4.99.11). | |
| 16 | xyloglucan: xyloglucosyltransferase (EC 2.4.1.207); keratan-sulfate | 1 |
| | endo-1,4-beta-galactosidase (EC 3.2.1.103); Glucan endo-1,3-beta-D- | |
| | glucosidase (EC 3.2.1.39); endo-1,3(4)-beta-glucanase (EC 3.21.6); | |
| | Licheninase (EC 3.2.1.73): agarase (EC 3.2.1.81 );betacarrageenase | |
| | (EC 3.2.1.83); xyioglucanase (EC 3.2.1.151) | |
| 18 | chitinase (EC 3.2.1.14); endo-beta-N-acetylglucosaminidase (EC | 6 |
| | 3.2.1.96); non-catalytic proteins: xylanase inhibitors; concanavalin B; | |
| | narbonin | |
| 19 | chitinase(EC 3.2.1.14). | 2 |
| 20 | beta-hexosaminidase (EC 3.2.1.52); lacto-N-biosidase (EC 3.2.1.140); - | 3 |
| | 1,6-N-acetylglucosaminidase) (EC 3.2.1.—) | |
| 25 | lysozyme(EC 3.2.1.17) | 1 |
| 26 | beta-mannanase (EC 3.2.1.78); beta-1,3-xylanase (EC 3.2.1.32) | 3 |
| 28 | polygalacturonase (EC 3.2.1.15); exo-polygalacturonase (EC 3.2.1.67); | 5 |
| | exo-polygalacturonosidase (EC 3.2.1.82); rhamnogalacturonase (EC | |
| | 3.2.1.—); endo-xylogalacturonan hydrolase (EC 3.2.1.—; | |
| | rhamnogalacturonan alpha-L-rhamnopyranohydrolase (EC 3.2.1.40) | |
| 29 | alpha-L-fucosidase (EC 3.2.1.51) | 3 |
| 30 | glucosylceramidase (EC 3.2.1.45); beta-1,6-glucanase (EC 3.2.1.75); | 2 |
| | beta-xylosidase (EC 3.2.1.37) | |
| 31 | alpha-glucosidase (EC 3.2.1.20): alpha-1,3-glucosidase (EC 3.2.1.84); | 3 |
| | sucrase-isomaltase (EC 3.2.1.48) (EC 3.2.1.10); alpha-xylosidase (EC | |
| | 3.2.1.—); alpha-glucan lyase (EC 4.2.2.13); isomaltosyltransferase (EC | |
| | 2.4.1.—). | |
| 36 | alpha-galactosidase (EC 3.2.1.22); alpha-N-acetylgalactosaminidase | 2 |
| | (EC 3.2.1.49); stachyose synthase (EC 2.4.1.67); raffinose synthase | |
| | (EC 2.4.1.82) | |
| 38 | alpha-mannosidase (EC 3.2.1.24); alpha-mannosidase (EC 3.2.1.114) | 1 |
| 43 | beta-xylosidase (EC 3.2.1.37); beta-1,3-xylosidase (EC 3.2.1.—); alpha- | 8 |
| | L-arabinofuranosidase (EC 3.2.1.55); arabinanase (EC 3.2.1.99); | |
| | xylanase (EC 3.2.1.8); galactan 1,3-beta-galactosidase (EC 3.2.1.145) | |
| 48 | endoglucanase (EC 3.2.1.4); chitinase (EC 3.2.1.14); | 1 |
| | cellobiohydrolases some cellobiohydrolases of this family have been | |
| | reported to act from the reducing ends of cellulose (EC 3.2.1.—), while | |
| | others have been reported to operate from the non-reducing ends to | |
| | liberate cellobiose or cellotriose or cellotetraose (EC 3.2.1.—). This | |
| | family also contains endo-processive celtulases (EC 3.2.1.—), whose | |
| | activity is hard to distinguish from that of cellobiohydrolases. | |
| 51 | alpha-L-arabinofuranosidase (EC 3.2.1.55); endoglucanase (EC 3.2.1.4) | 1 |
| 65 | trehalase (EC 3.2.1.28); maltose phosphorylase (EC 2.4.1.8); trehalose | 4 |
| | phosphorylase (EC 2.4.1.64); kojibiose phosphorylase (EC 2.4.1.230) | |
| 67 | alpha-glucuronidase (EC 3.2.1.139); xylan alpha-I,2-glucuronosidase | 1 |
| | (EC_3.2.1.131) | |
| 73 | peptidoglycan hydrolases with endo-beta-N-acetylglucosam inidase | 1 |
| | (EC 3.2.1.—) specificity; there is only one, unconfirmed, report of beta- | |
| | i,4-N-acetylmuramoylhydrolase (EC 3.2.1.17) activity | |
| 77 | amylomaltase or 4-aipha-glucanotransferase (EC 2.4.1.25) | 1 |
| 85 | endo-beta-N-acetylglucosaminidase (EC 3.2.1.96) | 1 |
| 87 | mycodextranase (EC 3.2.1.61); alpha-1,3-glucanase (EC 3.2.1.59) | 3 |
| 88 | d-4,5 unsaturated beta-glucuronyl hydrolase (EC 3.2.1.—) | 4 |
| 94 | cellobiose phosphorylase (EC 2.4.1.20); cellodextrin phosphorylase | 5 |
| | (EC 2.4.1.49); chitobiose phosphorylase (EC 2.4.1.—); cyclic beta-1,2- | |
| | glucan synthase (EC 2.4.1.—) | |
| 95 | alpha-1,2-L-fucosidase (EC 3.2.1.63); alpha-L-fucosidase (EC 3.2.1.51) | 2 |
| 105 | unsaturated rhamnogalacturonyl hydrolase (EC 3.2.1.—) | 3 |
| 106 | alpha-L-rhamnosidase (EC 3.2.1.40) | 1 |
| 112 | lacto-N-biose phosphorylase or galacto-N-biose phosphorylase (EC | 3 |
| | 2.4.1.211) | |

In one embodiment, enzymes that degrade polysaccharides are used for the pretreatment of biomass and can include enzymes that degrade cellulose, namely, cellulases. Examples of some cellulases include endocellulases (EC 3.2.1.4) and exo-cellulases (EC 3.2.1.91), and hydrolyze beta-1,4-glucosidic bonds. Members of the GH5, GQ.20 and GH48 families can have both exo- and endo-cellulase activity.

In one embodiment, enzymes that degrade polysaccharides are used for the pretreatment of biomass and can include enzymes that have the ability to degrade hemicellulose, namely, hemicellulases. Hemicellulose can be a major component of plant biomass and can contain a mixture of pentoses and hexoses, for example, D-xylopyranose, L-arabinofuranose, D-mannopyranose, D-glucopyranose, D-galactopyranose, D-glucopyranosyluronic acid and other sugars. In one embodiment, predicted hemicellulases identified in C. phytofermentans that can be used in the pretreatment of biomass include enzymes active on the linear backbone of hemicellulose, for example, endo-beta-1,4-D-xylanase (EC 3.2.1.8), such as GH5, GH10, GH11, and GH43 family members; 1,4-beta-D-xyloside xylohydrolase (EC 3.2.1.37), such as GH30, GH43, and GH3 family members; and beta-mannanase (EC 3.2.1.78), such as GH26 family members.

In one embodiment, enzymes that degrade polysaccharides are used for the pretreatment of biomass and can include enzymes that have the ability to degrade pectin, namely, pectinases. In plant cell walls, the cross-linked cellulose network can be embedded in a matrix of pectins that can be covalently cross-linked to xyloglucans and certain structural proteins. Pectin can comprise homogalacturonan (HG) or rhamnogalacturonan (RH).

In one embodiment, pretreatment of biomass includes enzymes that can hydrolyze starch. C. phytofermentans can degrade starch and chitin (Warnick, T. A. and Leschine, S. B. Clostridium phytofermentans sp. nov., a cellulolytic mesophile from forest soil. Int. J. Syst. Eva Microbiol. 52, 1155-1160 (2002); Leschine, S. B. in Handbook on Clostridia (ed Dane, P.) (CRC Press, Boca Raton, 2005); Reguera, G. & Leschine, S. B. Chitin degradation by cellulolytic anaerobes and facultative aerobes from soils and sediments. FEMS Micro biol. Lett. 204, 367-374 (2001)). Enzymes that hydrolyze starch include alpha-amylase, glucoamylase, beta-amylase, exo-alpha-1,4-glucanase, and pullulanase.

In one embodiment, pretreatment of biomass comprises hydrolases that can include enzymes that hydrolyze chitin. Examples of enzymes that can hydrolyze chitin include GH18 and GH19 family members.

In another embodiment, hydrolases can include enzymes that hydrolyze lichen, namely, lichenase, for example, GH16 family members.

In one embodiment, after pretreatment by any of the above methods the feedstock contains cellulose, hemicellulose, soluble oligomers, simple sugars, lignin, volatiles and ash. The parameters of the pretreatment can be changed to vary the concentration of the components of the pretreated feedstock. For example, in one embodiment, a pretreatment is chosen so that the concentration of soluble oligomers is high and the concentration of lignin is low after pretreatment. Examples of parameters of the pretreatment include temperature, pressure, time, and pH.

In one embodiment, the parameters of the pretreatment are changed to vary the concentration of the components of the pretreated feedstock such that concentration of the components in the pretreated stock is optimal for fermentation with a microorganism such as a Q.17, Q.18, Q.19 or Q.20 microorganism.

In one embodiment, the parameters of the pretreatment are changed to encourage the release of the components of a genetically modified feedstock such as enzymes stored within a vacuole to increase or complement the enzymes synthesized by Q.17, Q.18, Q.19 or Q.20 to produce optimal release of the fermentable components during hydrolysis and fermentation.

In one embodiment, the parameters of the pretreatment are changed such that concentration of accessible cellulose in the pretreated feedstock is 1%, 5%, 10%, 12%, 13%, 14%, 15%, 16%, 17%, 19%, 20%, 30%, 40% or 50%. In one embodiment, the parameters of the pretreatment are changed such that concentration of accessible cellulose in the pretreated feedstock is 5% to 30%. In one embodiment, the parameters of the pretreatment are changed such that concentration of accessible cellulose in the pretreated feedstock is 10% to 20%.

In one embodiment, the parameters of the pretreatment are changed such that concentration of hemicellulose in the pretreated feedstock is 1%, 5%, 10%, 12%, 13%, 14%, 15%, 16%, 17%, 19%, 20%, 21%, 22%, 23%, 24%, 25%, 26%, 27%, 28%, 29%, 30%, 40% or 50%. In one embodiment, the parameters of the pretreatment are changed such that concentration of hemicellulose in the pretreated feedstock is 5% to 40%. In one embodiment, the parameters of the pretreatment are changed such that concentration of hemicellulose in the pretreated feedstock is 10% to 30%.

In one embodiment, the parameters of the pretreatment are changed such that concentration of soluble oligomers in the pretreated feedstock is 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In one embodiment, the parameters of the pretreatment are changed such that concentration of soluble oligomers in the pretreated feedstock is 30% to 90%. In one embodiment, the parameters of the pretreatment are changed such that concentration of soluble oligomers in the pretreated feedstock is 45% to 80%. In one embodiment, the parameters of the pretreatment are changed such that concentration of soluble oligomers in the pretreated feedstock is 45% to 80% and the soluble oligomers are primarily cellobiose and xylobiose.

In one embodiment, the parameters of the pretreatment are changed such that concentration of simple sugars in the pretreated feedstock is 1%, 5%, 10%, 12%, 13%, 14%, 15%, 16%, 17%, 19%, 20%, 30%, 40% or 50%. In one embodiment, the parameters of the pretreatment are changed such that concentration of simple sugars in the pretreated feedstock is 0% to 20%. In one embodiment, the parameters of the pretreatment are changed such that concentration of simple sugars in the pretreated feedstock is 0% to 5%. Examples of simple sugars include, but are not limited to, C5 and C6 monomers and dimers (e.g., glucose, fructose, galactose, xylose, ribose, sucrose, lactose, cellobiose, maltose, lactulose, trehalose, isomaltose, sophorose, laminaribiose, maltulose, mannobiose, melibiose, melibiulose, xylobiose, etc.).

In one embodiment, the parameters of the pretreatment are changed such that concentration of lignin in the pretreated feedstock is 1%, 5%, 10%, 12%, 13%, 14%, 15%, 16%, 17%, 19%, 20%, 30%, 40% or 50%.

In one embodiment, the parameters of the pretreatment are changed such that concentration of lignin in the pretreated feedstock is 0% to 20%. In one embodiment, the parameters of the pretreatment are changed such that concentration of lignin in the pretreated feedstock is 0% to 5%. In one embodiment, the parameters of the pretreatment are changed such that concentration of lignin in the pretreated feedstock is less than 1% to 2%. In one embodiment, the parameters of the pretreatment are changed such that the concentration of phenolics is minimized.

In one embodiment, the parameters of the pretreatment are changed such that concentration of furfural and low molecular weight lignin in the pretreated feedstock is less than 10%, 9%, 8%, 7%, 6%, 5%, 4%, 3%, 2%, or 1%. In one embodiment, the parameters of the pretreatment are changed such that concentration of furfural and low molecular weight lignin in the pretreated feedstock is less than 1% to 2%.

In one embodiment, the parameters of the pretreatment are changed such that concentration of accessible cellulose is 10% to 20%, the concentration of hemicellulose is 10% to 30%, the concentration of soluble oligomers is 45% to 80%, the concentration of simple sugars is 0% to 5%, and the concentration of lignin is 0% to 5% and the concentration of furfural and low molecular weight lignin in the pretreated feedstock is less than 1% to 2%.

In one embodiment, the parameters of the pretreatment are changed to obtain a high concentration of hemicellulose and a low concentration of lignin. In one embodiment, the parameters of the pretreatment are changed to obtain a high concentration of hemicellulose and a low concentration of lignin such that concentration of the components in the pretreated stock is optimal for fermentation with a microorganism such as Q.17, Q.18, Q.19 or Q.20.

### Recovery of Ethanol or Other Fermentation End Products

In another aspect, methods are provided for the recovery of the fermentation end products, such as an alcohol (e.g. ethanol, propanol, methanol, butanol, etc.) another biofuel or chemical product. In one embodiment, broth will be harvested at some point during the fermentation, and fermentation end product or products will be recovered. The broth with ethanol to be recovered will include both ethanol and impurities. The impurities include materials such as water, cell bodies, cellular debris, excess carbon substrate, excess nitrogen substrate, other remaining nutrients, non-ethanol metabolites, and other medium components or digested medium components. During the course of processing the broth, the broth can be heated and/or reacted with various reagents, resulting in additional impurities in the broth.

In one embodiment, the processing steps to recover ethanol frequently includes several separation steps, including, for example, distillation of a high concentration ethanol material from a less pure ethanol-containing material. In one embodiment, the high concentration ethanol material can be further concentrated to achieve very high concentration ethanol, such as 98% or 99% or 99.5% (wt.) or even higher. Other separation steps, such as filtration, centrifugation, extraction, adsorption, etc. can also be a part of some recovery processes for ethanol as a product or biofuel, or other biofuels or chemical products.

In one embodiment, a process can be scaled to produce commercially useful biofuels. In another embodiment, Q.17, Q.18, Q.19 or Q.20 is used to produce an alcohol, e.g., ethanol, butanol, propanol, methanol, or a fuel such as hydrocarbons hydrogen, methane, and hydroxy compounds. In another embodiment, Q.17, Q.18, Q.19 or Q.20 is used to produce a carbonyl compound such as an aldehyde or ketone (e.g. acetone, formaldehyde, 1-propanal, etc.), an organic acid, a derivative of an organic acid such as an ester (e.g. wax ester, glyceride, etc.), 1,2-propanediol, 1,3-propanediol, lactic acid, formic acid, acetic acid, succinic acid, pyruvic acid, or an enzyme such as a cellulase, polysaccharase, lipases, protease, ligninase, and hemicellulose.

In one embodiment, a fed-batch fermentation for production of fermentation end product is described. In another embodiment, a fed-batch fermentation for production of ethanol is described. Fed-batch culture is a kind of microbial process in which medium components, such as carbon substrate, nitrogen substrate, vitamins, minerals, growth factors, cofactors, etc. or biocatalysts (including, for example, fresh organisms, enzymes prepared by Q.17, Q.18, Q.19 or Q.20 in a separate fermentation, enzymes prepared by other organisms, or a combination of these) are supplied to the fermentor during cultivation, but culture broth is not harvested at the same time and volume. To improve bioconversion from soluble and insoluble substrates, such as those that can be used in biofuels production, various feeding strategies can be utilized to improve yields and/or productivity. This technique can be used to achieve a high cell density within a given time. It can also be used to maintain a good supply of nutrients and substrates for the bioconversion process. It can also be used to achieve higher titer and productivity of desirable products that might otherwise be achieved more slowly or not at all.

In another embodiment, the feeding strategy balances the cell production rate and the rate of hydrolysis of the biomass feedstock with the production of ethanol. Sufficient medium components are added in quantities to achieved sustained cell production and hydrolysis of the biomass feedstock with production of ethanol. In one embodiment, sufficient carbon and nitrogen substrate are added in quantities to achieve sustained production of fresh cells and hydrolytic enzymes for conversion of polysaccharides into lower sugars as well as sustained conversion of the lower sugars into fresh cells and ethanol.

In another embodiment, the level of a medium component is maintained at a desired level by adding additional medium component as the component is consumed or taken up by the organism. Examples of medium components include, but are not limited to, carbon substrate, nitrogen substrate, vitamins, minerals, growth factors, cofactors, and biocatalysts. The medium component can be added continuously or at regular or irregular intervals. In one embodiment, additional medium component is added prior to the complete depletion of the medium component in the medium. In one embodiment, complete depletion can effectively be used, for example to initiate different metabolic pathways, to simplify downstream operations, or for other reasons as well. In one embodiment, the medium component level is allowed to vary by about 10% around a midpoint, in one embodiment, it is allowed to vary by about 30% around a midpoint, and in one embodiment, it is allowed to vary by 60% or more around a midpoint. In one embodiment, the medium component level is maintained by allowing the medium component to be depleted to an appropriate level, followed by increasing the medium component level to another appropriate level. In one embodiment, a medium component, such as a vitamin component, is added at two different time points during fermentation process. For example, one-half of a total amount of vitamin is added at the beginning of fermentation and the other half is added at midpoint of fermentation.

In another embodiment, the nitrogen level is maintained at a desired level by adding additional nitrogen-containing material as nitrogen is consumed or taken up by the organism. The nitrogen-containing material can be added continuously or at regular or irregular intervals. In one embodiment, additional nitrogen-containing material is added prior to the complete depletion of the nitrogen available in the medium. In one embodiment, complete depletion can effectively be used, for example to initiate different metabolic pathways, to simplify downstream operations, or for other reasons as well. In one embodiment, the nitrogen level (as measured by the grams of actual nitrogen in the nitrogen-containing material per liter of broth) is allowed to vary by about 10% around a midpoint, in some embodiments, it is allowed to vary by about 30% around a midpoint, and in some embodiments, it is allowed to vary by 60% or more around a midpoint. In one embodiment, the nitrogen level is maintained by allowing the nitrogen to be depleted to an appropriate level, followed by increasing the nitrogen level to another appropriate level. Useful nitrogen levels include levels of about 5 to about 10 g/L. In one embodiment, levels of about 1 to about 12 g/L can also be usefully employed. In another embodiment, levels, such as about 0.5, 0.1 g/L or even lower, and higher levels, such as about 20, 30 g/L or even higher are used. In another embodiment, a useful nitrogen level is about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22 23, 24, 25, 26, 27, 28, 29 or 30 g/L. Such nitrogen levels can facilitate the production of fresh cells and of hydrolytic enzymes. Increasing the level of nitrogen can lead to higher levels of enzymes and/or greater production of cells, and result in higher productivity of desired products. Nitrogen can be supplied as a simple nitrogen-containing material, such as an ammonium compounds (e.g. ammonium sulfate, ammonium hydroxide, ammonia, ammonium nitrate, or any other compound or mixture containing an ammonium moiety), nitrate or nitrite compounds (e.g. potassium, sodium, ammonium, calcium, or other compound or mixture containing a nitrate or nitrite moiety), or as a more complex nitrogen-containing material, such as amino acids, proteins, hydrolyzed protein, hydrolyzed yeast, yeast extract, dried brewer's yeast, yeast hydrolysates, distillers' grains, soy protein, hydrolyzed soy protein, fermentation products, and processed or corn steep powder or unprocessed protein-rich vegetable or animal matter, including those derived from bean, seeds, soy, legumes, nuts, milk, pig, cattle, mammal, fish, as well as other parts of plants and other types of animals. Nitrogen-containing materials useful in various embodiments also include materials that contain a nitrogen-containing material, including, but not limited to mixtures of a simple or more complex nitrogen-containing material mixed with a carbon source, another nitrogen-containing material, or other nutrients or non-nutrients, and AFEX treated plant matter.

In another embodiment, the carbon level is maintained at a desired level by adding sugar compounds or material containing sugar compounds (“Sugar-Containing Material”) as sugar is consumed or taken up by the organism. The sugar-containing material can be added continuously or at regular or irregular intervals. In one embodiment, additional sugar-containing material is added prior to the complete depletion of the sugar compounds available in the medium. In one embodiment, complete depletion can effectively be used, for example to initiate different metabolic pathways, to simplify downstream operations, or for other reasons as well. In one embodiment, the carbon level (as measured by the grams of sugar present in the sugar-containing material per liter of broth) is allowed to vary by about 10% around a midpoint, in one embodiment, it is allowed to vary by about 30% around a midpoint, and in one embodiment, it is allowed to vary by 60% or more around a midpoint. In one embodiment, the carbon level is maintained by allowing the carbon to be depleted to an appropriate level, followed by increasing the carbon level to another appropriate level. In some embodiments, the carbon level can be maintained at a level of about 5 to about 120 g/L. However, levels of about 30 to about 100 g/L can also be usefully employed as well as levels of about 60 to about 80 g/L. In one embodiments, the carbon level is maintained at greater than 25 g/L for a portion of the culturing. In another embodiment, the carbon level is maintained at about 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 101 g/L, 102 g/L, 103 g/L, 104 g/L, 105 g/L, 106 g/L, 107 g/L, 108 g/L, 109 g/L, 110 g/L, 111 g/L, 112 g/L, 113 g/L, 114 g/L, 115 g/L, 116 g/L, 117 g/L, 118 g/L, 119 g/L, 120 g/L, 121 g/L, 122 g/L, 123 g/L, 124 g/L, 125 g/L, 126 g/L, 127 g/L, 128 g/L, 129 g/L, 130 g/L, 131 g/L, 132 g/L, 133 g/L, 134 g/L, 135 g/L, 136 g/L, 137 g/L, 138 g/L, 139 g/L, 140 g/L, 141 g/L, 142 g/L, 143 g/L, 144 g/L, 145 g/L, 146 g/L, 147 g/L, 148 g/L, 149 g/L, or 150 g/L.

One advantage of using a biocatalyst such as Q.17, Q.18, Q.19 or Q.20 is the ability of any of these strains to hydrolyze cellulosic substrates to oligomers and/or monomers in the presence of normal repressors. One such repressor is glucose. As glucose concentrations increase from hydrolysis of cellulose and other polymers during the fermentation, a biocatalyst is not able to convert this monomer to ethanol fast enough to prevent feedback inhibition of cellulase synthesis. Thus, polymer hydrolysis decreases and may even stop, slowing the overall rate of ethanol production. Deregulation of cellulase synthesis solves this problem as the carbon catabolite repression mechanism is unable to regulate the cellulolytic system of Q.17, Q.18, Q.19 or Q.20 strains of Clostridium phytofermentans.

The carbon substrate, like the nitrogen substrate, can be necessary for cell production and enzyme production, but unlike the nitrogen substrate, it can serve as the raw material for ethanol. Frequently, more carbon substrate can lead to greater production of ethanol. In another embodiment, it can be advantageous to operate with the carbon level and nitrogen level related to each other for at least a portion of the fermentation time. In one embodiment, the ratio of carbon to nitrogen is maintained within a range of about 30:1 to about 10:1. In another embodiment, the ratio of carbon nitrogen is maintained from about 20:1 to about 10:1 or more preferably from about 15:1 to about 10:1. In another embodiment, the ratio of carbon nitrogen is about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1.

Maintaining the ratio of carbon and nitrogen ratio within particular ranges can result in benefits to the operation such as the rate of hydrolysis of carbon substrate, which depends on the amount of carbon substrate and the amount and activity of enzymes present, being balanced to the rate of ethanol production. Such balancing can be important, for example, due to the possibility of inhibition of cellular activity due to the presence of a high concentration of low molecular weight saccharides, and the need to maintain enzymatic hydrolytic activity throughout the period where longer chain saccharides are present and available for hydrolysis. Balancing the carbon to nitrogen ratio can, for example, facilitate the sustained production of these enzymes such as to replace those which have lost activity.

In another embodiment, the amount and/or timing of carbon, nitrogen, or other medium component addition can be related to measurements taken during the fermentation. For example, the amount of monosaccharides present, the amount of insoluble polysaccharide present, the polysaccharase activity, the amount of ethanol present, the amount of cellular material (for example, packed cell volume, dry cell weight, etc.) and/or the amount of nitrogen (for example, nitrate, nitrite, ammonia, urea, proteins, amino acids, etc.) present can be measured. The concentration of the particular species, the total amount of the species present in the fermentor, the number of hours the fermentation has been running, and the volume of the fermentor can be considered. In various embodiments, these measurements can be compared to each other and/or they can be compared to previous measurements of the same parameter previously taken from the same fermentation or another fermentation. Adjustments to the amount of a medium component can be accomplished such as by changing the flow rate of a stream containing that component or by changing the frequency of the additions for that component. In one embodiment, the amount of polysaccharide can be reduced when the monosaccharides level increases faster than the ethanol level increases. In another embodiment, the amount of polysaccharide can be increased when the amount or level of monosaccharides decreases while the ethanol production approximately remains steady. In another embodiment, the amount of nitrogen can be increased when the monosaccharides level increases faster than the viable cell level. The amount of polysaccharide can also be increased when the cell production increases faster than the ethanol production. In another embodiment, the amount of nitrogen can be increased when the enzyme activity level decreases.

In another embodiment, different levels or complete depletion of a medium component can effectively be used, for example to initiate different metabolic pathways or to change the yield of the different products of the fermentation process. For instance, different levels or complete depletion of a medium component can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, different levels or complete depletion of nitrogen can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, different levels or complete depletion of carbon can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, the ratio of carbon level to nitrogen level for at least a portion of the fermentation time can effectively be used to increase the ethanol yield and productivity, to improve carbon utilization (e.g., g ethanol/g sugar fermented) and reduced acid production (e.g., g acid/g ethanol and g acid/g sugar fermented).

In another embodiment, a fed batch operation can be employed, wherein medium components and/or fresh cells are added during the fermentation without removal of a portion of the broth for harvest prior to the end of the fermentation. In one embodiment, a fed-batch process is based on feeding a growth limiting nutrient medium to a culture of microorganisms. In one embodiment, the feed medium is highly concentrated to avoid dilution of the bioreactor. In another embodiment, the controlled addition of the nutrient directly affects the growth rate of the culture and avoids overflow metabolism such as the formation of side metabolites. In one embodiment, the growth limiting nutrient is a nitrogen source or a saccharide source.

In another embodiment, a modified fed batch operation can be employed wherein a portion of the broth is harvested at discrete times. Such a modified fed batch operation can be advantageously employed when, for example, very long fermentation cycles are employed. Under very long fermentation conditions, the volume of liquid inside the fermentor increases. In order to operate for very long periods, it can be advantageous to partially empty the fermentor, for example, when the volume is nearly full. A partial harvest of broth followed by supplementation with fresh medium ingredients, such as with a fed batch operation, can improve fermentor utilization and can facilitate higher plant throughputs due to a reduction in the time for tasks such as cleaning and sterilization of equipment. When the “partial harvest” type of operation is employed, the fermentation can be seeded with the broth that remains in the fermentor, or with fresh inoculum, or with a mixture of the two. In addition, broth can be recycled for use as fresh inoculum either alone or in combination with other fresh inoculum.

In one embodiment, a fed batch operation can be employed, wherein medium components and/or fresh cells are added during the fermentation when the hydrolytic activity of the broth has decreased. In one embodiment, medium components and/or fresh cells are added during the fermentation when the hydrolytic activity of the broth has decreased about 5%, 10%, 15%, 20%, 25%, 30%, 35%, 40%, 45%, 50%, 55%, 60%, 65%, 75%, 80%, 85%, 90%, 95%, or 100%.

While Q.17, Q.18, Q.19 or Q.20 can be used in long or short fermentation cycles, it is particularly well-suited for long fermentation cycles and for use in fermentations with partial harvest, self-seeding, and broth recycle operations due to the anaerobic conditions of the fermentation, the presence of alcohol, the very fast growth rate of the strains compared to other Clostridia, and, in one embodiment, the use of a solid carbon substrate, whether or not resulting in low sugar concentrations in the broth.

In another embodiment, a fermentation to produce ethanol is performed by culturing a strain of Q.17, Q.18, Q.19 or Q.20 in a medium having a high concentration of one or more carbon sources, and/or augmenting the culture with addition of fresh cells of Q.17, Q.18, Q.19 or Q.20 during the course of the fermentation. The resulting production of ethanol can be up to 1-fold, 2-fold, 3-fold, 4-fold, 5-fold, 6-fold, 7-fold, 8-fold, 9-fold, and in some cases up to 10-fold and higher in volumetric productivity than a batch process and achieve a carbon conversion efficiency approaching the theoretical maximum. The theoretical maximum can vary with the substrate and product. For example, the generally accepted maximum efficiency for conversion of glucose to ethanol is 0.51 g ethanol/g glucose. In one embodiment, Q.17, Q.18, Q.19 or Q.20 can produce about 40-100% of a theoretical maximum yield of ethanol. In another embodiment, 0.17, Q.18, Q.19 or Q.20 can produce up to about 40% of the theoretical maximum yield of ethanol. In another embodiment, Q.17, Q.18, Q.19 or Q.20 can produce up to about 50% of the theoretical maximum yield of ethanol. In another embodiment, Q.17, Q.18, Q.19 or Q.20 can produce about 70% of the theoretical maximum yield of ethanol. In another embodiment, Q.17, Q.18, Q.19 or Q.20 can produce about 90% of the theoretical maximum yield of ethanol. In another embodiment, Q.17, Q.18, Q.19 or Q.20 can produce about 95% of the theoretical maximum yield of ethanol. In another embodiment, Q.17, Q.18, Q.19 or Q.20 can produce about 95% of the theoretical maximum yield of ethanol. In another embodiment, Q.17, Q.18, Q.19 or Q.20 can produce about 99% of the theoretical maximum yield of ethanol. In another embodiment, 0.17, Q.18, Q.19 or Q.20 can produce about 100% of the theoretical maximum yield of ethanol. In one embodiment, Q.17, Q.18, Q.19 or Q.20 can produce up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.99%, or 100% of a theoretical maximum yield of ethanol.

Q.17, Q.18, Q.19 or Q.20 cells used for the seed inoculum or for cell augmentation can be prepared or treated in ways that relate to their ability to produce enzymes useful for hydrolyzing the components of the production medium. For example, in one embodiment, the cells can produce useful enzymes after they are transferred to the production medium or production fermentor. In another embodiment, Q.17, Q.18, Q.19 or Q.20 cells can have already produced useful enzymes prior to transfer to the production medium or the production fermentor. In another embodiment, Q.17, Q.18, Q.19 or Q.20 cells can be ready to produce useful enzymes once transferred to the production medium or the production fermentor, or Q.17, Q.18, Q.19 or Q.20 cells can have some combination of these enzyme production characteristics. In one embodiment, the seed can be grown initially in a medium containing a simple sugar source, such as corn syrup or dried brewer's yeast, and then transitioned to the production medium carbon source prior to transfer to the production medium. In another embodiment, the seed is grown on a combination of simple sugars and production medium carbon source prior to transfer to the production medium. In another embodiment, the seed is grown on the production medium carbon source from the start. In another embodiment, the seed is grown on one production medium carbon source and then transitioned to another production medium carbon source prior to transfer to the production medium. In another embodiment, the seed is grown on a combination of production medium carbon sources prior to transfer to the production medium. In another embodiment, the seed is grown on a carbon source that favors production of hydrolytic enzymes with activity toward the components of the production medium.

In another embodiment, a fermentation to produce ethanol is performed by culturing a strain of Q.17, Q.18, Q.19 or Q.20 microorganism and adding fresh medium components and fresh Q.17, Q.18, Q.19 or Q.20 cells while the cells in the fermentor are growing. Medium components, such as carbon, nitrogen, and combinations of these, can be added as disclosed herein, as well as other nutrients, including vitamins, factors, cofactors, enzymes, minerals, salts, and such, sufficient to maintain an effective level of these nutrients in the medium. The medium and Q.17, Q.18, Q.19 or Q.20 can be added simultaneously, or one at a time. In another embodiment, fresh Q.17, Q.18, Q.19 or Q.20 cells can be added when hydrolytic enzyme activity decreases, especially when the activity of those hydrolytic enzymes that are more sensitive to the presence of alcohol decreases. After the addition of fresh Q.17, Q.18, Q.19 or Q.20 cells, a nitrogen feed or a combination of nitrogen and carbon feed and/or other medium components can be fed, prolonging the enzymatic production or other activity of the cells. In another embodiment, the cells can be added with sufficient carbon and nitrogen to prolong the enzymatic production or other activity of the cells sufficiently until the next addition of fresh cells. In another embodiment, fresh Q.17, Q.18, Q.19 or Q.20 cells can be added when both the nitrogen level and carbon level present in the fermentor increase. In another embodiment, Q.17, Q.18, Q.19 or Q.20 cells can be added when the viable cell count decreases, especially when the nitrogen level is relatively stable or increasing. In another embodiment, fresh cells can be added when a significant portion of the viable cells are in the process of sporulation, or have sporulated. Appropriate times for adding fresh Q.17, Q.18, Q.19 or Q.20 cells can be when the portion of cells in the process of sporulation or have sporulated is about 2% to about 100%, about 10% to about 75%, about 20% to about 50%, or about 25% to about 30% of the cells are in the process of sporulation or have sporulated.

### Medium Compositions

In various embodiments, particular medium components can have beneficial effects on the performance of the fermentation, such as increasing the titer of desired products, or increasing the rate that the desired products are produced. Specific compounds can be supplied as a specific, pure ingredient, such as a particular amino acid, or it can be supplied as a component of a more complex ingredient, such as using a microbial, plant or animal product as a medium ingredient to provide a particular amino acid, promoter, cofactor, or other beneficial compound. In some cases, the particular compound supplied in the medium ingredient can be combined with other compounds by the organism resulting in a fermentation-beneficial compound. One example of this situation would be where a medium ingredient provides a specific amino acid which the organism uses to make an enzyme beneficial to the fermentation. Other examples can include medium components that are used to generate growth or product promoters, etc. In such cases, it can be possible to obtain a fermentation-beneficial result by supplementing the enzyme, promoter, growth factor, etc. or by adding the precursor. In some situations, the specific mechanism whereby the medium component benefits the fermentation is not known, only that a beneficial result is achieved.

In one embodiment, beneficial fermentation results can be achieved by adding yeast extract. The addition of the yeast extract can result in increased ethanol titer in batch fermentation, improved productivity reduced production of side products such as organic acids. In one embodiment, beneficial results with yeast extract can be achieved at usage levels of about 0.5 to about 50 g/L, about 5 to about 30 g/L, or about 10 to about 30 g/L. The yeast extract can also be fed throughout the course of the entire fermentation or a portion of the fermentation, continuously or delivered at intervals.

In one embodiment, usage levels include maintaining a nitrogen concentration of about 0.05 g/L to about 3 g/L (as nitrogen), where at least a portion of the nitrogen is supplied from corn steep powder; or about 0.3 g/L to 1.3 g/L; or 0.4 g/L to about 0.9 g/L. In another embodiment, the nitrogen concentration is about 0.05 g/L, 0.06 g/L, 0.07 g/L, 0.08 g/L, 0.09 g/L, 0.1 g/L, 0.11 g/L, 0.12 g/L, 0.13 g/L, 0.14 g/L, 0.15 g/L, 0.16 g/L, 0.17 g/L, 0.18 g/L, 0.19 g/L, 0.2 g/L, 0.21 g/L, 0.22 g/L, 0.23 g/L, 0.24 g/L, 0.25 g/L, 0.26 g/L, 0.27 g/L, 0.28 g/L, 0.29 g/L, 0.3 g/L, 0.31 g/L, 0.32 g/L, 0.33 g/L, 0.34 g/L, 0.35 g/L, 0.36 g/L, 0.37 g/L, 0.38 g/L, 0.39 g/L, 0.4 g/L, 0.41 g/L, 0.42 g/L, 0.43 g/L, 0.44 g/L, 0.45 g/L, 0.46 g/L, 0.47 g/L, 0.48 g/L, 0.49 g/L, 0.5 g/L, 0.51 g/L, 0.52 g/L, 0.53 g/L, 0.54 g/L, 0.55 g/L, 0.56 g/L, 0.57 g/L, 0.58 g/L, 0.59 g/L, 0.6 g/L, 0.61 g/L, 0.62 g/L, 0.63 g/L, 0.64 g/L, 0.65 g/L, 0.66 g/L, 0.67 g/L, 0.68 g/L, 0.69 g/L, 0.7 g/L, 0.71 g/L, 0.72 g/L, 0.73 g/L, 0.74 g/L, 0.75 g/L, 0.76 g/L, 0.77 g/L, 0.78 g/L, 0.79 g/L, 0.8 g/L, 0.81 g/L, 0.82 g/L, 0.83 g/L, 0.84 g/L, 0.85 g/L, 0.86 g/L, 0.87 g/L, 0.88 g/L, 0.89 g/L, 0.9 g/L, 0.91 g/L, 0.92 g/L, 0.93 g/L, 0.94 g/L, 0.95 g/L, 0.96 g/L, 0.97 g/L, 0.98 g/L, 0.99 g/L, 1 g/L, 1.01 g/L, 1.02 g/L, 1.03 g/L, 1.04 g/L, 1.05 g/L, 1.06 g/L, 1.07 g/L, 1.08 g/L, 1.09 g/L, 1.1 g/L, 1.11 g/L, 1.12 g/L, 1.13 g/L, 1.14 g/L, 1.15 g/L, 1.16 g/L, 1.17 g/L, 1.18 g/L, 1.19 g/L, 1.2 g/L, 1.21 g/L, 1.22 g/L, 1.23 g/L, 1.24 g/L, 1.25 g/L, 1.26 g/L, 1.27 g/L, 1.28 g/L, 1.29 g/L, 1.3 g/L, 1.31 g/L, 1.32 g/L, 1.33 g/L, 1.34 g/L, 1.35 g/L, 1.36 g/L, 1.37 g/L, 1.38 g/L, 1.39 g/L, 1.4 g/L, 1.41 g/L, 1.42 g/L, 1.43 g/L, 1.44 g/L, 1.45 g/L, 1.46 g/L, 1.47 g/L, 1.48 g/L, 1.49 g/L, 1.5 g/L, 1.51 g/L, 1.52 g/L, 1.53 g/L, 1.54 g/L, 1.55 g/L, 1.56 g/L, 1.57 g/L, 1.58 g/L, 1.59 g/L, 1.6 g/L, 1.61 g/L, 1.62 g/L, 1.63 g/L, 1.64 g/L, 1.65 g/L, 1.66 g/L, 1.67 g/L, 1.68 g/L, 1.69 g/L, 1.7 g/L, 1.71 g/L, 1.72 g/L, 1.73 g/L, 1.74 g/L, 1.75 g/L, 1.76 g/L, 1.77 g/L, 1.78 g/L, 1.79 g/L, 1.8 g/L, 1.81 g/L, 1.82 g/L, 1.83 g/L, 1.84 g/L, 1.85 g/L, 1.86 g/L, 1.87 g/L, 1.88 g/L, 1.89 g/L, 1.9 g/L, 1.91 g/L, 1.92 g/L, 1.93 g/L, 1.94 g/L, 1.95 g/L, 1.96 g/L, 1.97 g/L, 1.98 g/L, 1.99 g/L, 2 g/L, 2.01 g/L, 2.02 g/L, 2.03 g/L, 2.04 g/L, 2.05 g/L, 2.06 g/L, 2.07 g/L, 2.08 g/L, 2.09 g/L, 2.1 g/L, 2.11 g/L, 2.12 g/L, 2.13 g/L, 2.14 g/L, 2.15 g/L, 2.16 g/L, 2.17 g/L, 2.18 g/L, 2.19 g/L, 2.2 g/L, 2.21 g/L, 2.22 g/L, 2.23 g/L, 2.24 g/L, 2.25 g/L, 2.26 g/L, 2.27 g/L, 2.28 g/L, 2.29 g/L, 2.3 g/L, 2.31 g/L, 2.32 g/L, 2.33 g/L, 2.34 g/L, 2.35 g/L, 2.36 g/L, 2.37 g/L, 2.38 g/L, 2.39 g/L, 2.4 g/L, 2.41 g/L, 2.42 g/L, 2.43 g/L, 2.44 g/L, 2.45 g/L, 2.46 g/L, 2.47 g/L, 2.48 g/L, 2.49 g/L, 2.5 g/L, 2.51 g/L, 2.52 g/L, 2.53 g/L, 2.54 g/L, 2.55 g/L, 2.56 g/L, 2.57 g/L, 2.58 g/L, 2.59 g/L, 2.6 g/L, 2.61 g/L, 2.62 g/L, 2.63 g/L, 2.64 g/L, 2.65 g/L, 2.66 g/L, 2.67 g/L, 2.68 g/L, 2.69 g/L, 2.7 g/L, 2.71 g/L, 2.72 g/L, 2.73 g/L, 2.74 g/L, 2.75 g/L, 2.76 g/L, 2.77 g/L, 2.78 g/L, 2.79 g/L, 2.8 g/L, 2.81 g/L, 2.82 g/L, 2.83 g/L, 2.84 g/L, 2.85 g/L, 2.86 g/L, 2.87 g/L, 2.88 g/L, 2.89 g/L, 2.9 g/L, 2.91 g/L, 2.92 g/L, 2.93 g/L, 2.94 g/L, 2.95 g/L, 2.96 g/L, 2.97 g/L, 2.98 g/L, 2.99 g/L, or 3 g/L.

In one embodiment, beneficial fermentation results can be achieved by adding corn steep powder to the fermentation. The addition of the corn steep powder can result in increased ethanol titer in batch fermentation, improved productivity and reduced production of side products such as organic acids. In another embodiment, beneficial results with corn steep powder can be achieved at usage levels of about 3 to about 20 g/L, about 5 to about 15 g/L, or about 8 to about 12 g/L. In another embodiment, beneficial results with steep powder can be achieved at a level of about 3 g/L, 3.1 g/L, 3.2 g/L, 3.3 g/L, 3.4 g/L, 3.5 g/L, 3.6 g/L, 3.7 g/L, 3.8 g/L, 3.9 g/L, 4 g/L, 4.1 g/L, 4.2 g/L, 4.3 g/L, 4.4 g/L, 4.5 g/L, 4.6 g/L, 4.7 g/L, 4.8 g/L, 4.9 g/L, 5 g/L, 5.1 g/L, 5.2 g/L, 5.3 g/L, 5.4 g/L, 5.5 g/L, 5.6 g/L, 5.7 g/L, 5.8 g/L, 5.9 g/L, 6 g/L, 6.1 g/L, 6.2 g/L, 6.3 g/L, 6.4 g/L, 6.5 g/L, 6.6 g/L, 6.7 g/L, 6.8 g/L, 6.9 g/L, 7 g/L, 7.1 g/L, 7.2 g/L, 7.3 g/L, 7.4 g/L, 7.5 g/L, 7.6 g/L, 7.7 g/L, 7.8 g/L, 7.9 g/L, 8 g/L, 8.1 g/L, 8.2 g/L, 8.3 g/L, 8.4 g/L, 8.5 g/L, 8.6 g/L, 8.7 g/L, 8.8 g/L, 8.9 g/L, 9 g/L, 9.1 g/L, 9.2 g/L, 9.3 g/L, 9.4 g/L, 9.5 g/L, 9.6 g/L, 9.7 g/L, 9.8 g/L, 9.9 g/L, 10 g/L, 10.1 g/L, 10.2 g/L, 10.3 g/L, 10.4 g/L, 10.5 g/L, 10.6 g/L, 10.7 g/L, 10.8 g/L, 10.9 g/L, 11 g/L, 11.1 g/L, 11.2 g/L, 11.3 g/L, 11.4 g/L, 11.5 g/L, 11.6 g/L, 11.7 g/L, 11.8 g/L, 11.9 g/L, 12 g/L, 12.1 g/L, 12.2 g/L, 12.3 g/L, 12.4 g/L, 12.5 g/L, 12.6 g/L, 12.7 g/L, 12.8 g/L, 12.9 g/L, 13 g/L, 13.1 g/L, 13.2 g/L, 13.3 g/L, 13.4 g/L, 13.5 g/L, 13.6 g/L, 13.7 g/L, 13.8 g/L, 13.9 g/L, 14 g/L, 14.1 g/L, 14.2 g/L, 14.3 g/L, 14.4 g/L, 14.5 g/L, 14.6 g/L, 14.7 g/L, 14.8 g/L, 14.9 g/L, 15 g/L, 15.1 g/L, 15.2 g/L, 15.3 g/L, 15.4 g/L, 15.5 g/L, 15.6 g/L, 15.7 g/L, 15.8 g/L, 15.9 g/L, 16 g/L, 16.1 g/L, 16.2 g/L, 16.3 g/L, 16.4 g/L, 16.5 g/L, 16.6 g/L, 16.7 g/L, 16.8 g/L, 16.9 g/L, 17 g/L, 17.1 g/L, 17.2 g/L, 17.3 g/L, 17.4 g/L, 17.5 g/L, 17.6 g/L, 17.7 g/L, 17.8 g/L, 17.9 g/L, 18 g/L, 18.1 g/L, 18.2 g/L, 18.3 g/L, 18.4 g/L, 18.5 g/L, 18.6 g/L, 18.7 g/L, 18.8 g/L, 18.9 g/L, 19 g/L, 19.1 g/L, 19.2 g/L, 19.3 g/L, 19.4 g/L, 19.5 g/L, 19.6 g/L, 19.7 g/L, 19.8 g/L, 19.9 g/L, or 20 g/L.

In one embodiment, corn steep powder can also be fed throughout the course of the entire fermentation or a portion of the fermentation, continuously or delivered at intervals. In another embodiment, usage levels include maintaining a nitrogen concentration of about 0.05 g/L to about 3 g/L (as nitrogen), where at least a portion of the nitrogen is supplied from corn steep powder; about 0.3 g/L to 1.3 g/L; or about 0.4 g/L to about 0.9 g/L. In another embodiment, the nitrogen level is about 0.05 g/L, 0.06 g/L, 0.07 g/L, 0.08 g/L, 0.09 g/L, 0.1 g/L, 0.11 g/L, 0.12 g/L, 0.13 g/L, 0.14 g/L, 0.15 g/L, 0.16 g/L, 0.17 g/L, 0.18 g/L, 0.19 g/L, 0.2 g/L, 0.21 g/L, 0.22 g/L, 0.23 g/L, 0.24 g/L, 0.25 g/L, 0.26 g/L, 0.27 g/L, 0.28 g/L, 0.29 g/L, 0.3 g/L, 0.31 g/L, 0.32 g/L, 0.33 g/L, 0.34 g/L, 0.35 g/L, 0.36 g/L, 0.37 g/L, 0.38 g/L, 0.39 g/L, 0.4 g/L, 0.41 g/L, 0.42 g/L, 0.43 g/L, 0.44 g/L, 0.45 g/L, 0.46 g/L, 0.47 g/L, 0.48 g/L, 0.49 g/L, 0.5 g/L, 0.51 g/L, 0.52 g/L, 0.53 g/L, 0.54 g/L, 0.55 g/L, 0.56 g/L, 0.57 g/L, 0.58 g/L, 0.59 g/L, 0.6 g/L, 0.61 g/L, 0.62 g/L, 0.63 g/L, 0.64 g/L, 0.65 g/L, 0.66 g/L, 0.67 g/L, 0.68 g/L, 0.69 g/L, 0.7 g/L, 0.71 g/L, 0.72 g/L, 0.73 g/L, 0.74 g/L, 0.75 g/L, 0.76 g/L, 0.77 g/L, 0.78 g/L, 0.79 g/L, 0.8 g/L, 0.81 g/L, 0.82 g/L, 0.83 g/L, 0.84 g/L, 0.85 g/L, 0.86 g/L, 0.87 g/L, 0.88 g/L, 0.89 g/L, 0.9 g/L, 0.91 g/L, 0.92 g/L, 0.93 g/L, 0.94 g/L, 0.95 g/L, 0.96 g/L, 0.97 g/L, 0.98 g/L, 0.99 g/L, 1 g/L, 1.01 g/L, 1.02 g/L, 1.03 g/L, 1.04 g/L, 1.05 g/L, 1.06 g/L, 1.07 g/L, 1.08 g/L, 1.09 g/L, 1.1 g/L, 1.11 g/L, 1.12 g/L, 1.13 g/L, 1.14 g/L, 1.15 g/L, 1.16 g/L, 1.17 g/L, 1.18 g/L, 1.19 g/L, 1.2 g/L, 1.21 g/L, 1.22 g/L, 1.23 g/L, 1.24 g/L, 1.25 g/L, 1.26 g/L, 1.27 g/L, 1.28 g/L, 1.29 g/L, 1.3 g/L, 1.31 g/L, 1.32 g/L, 1.33 g/L, 1.34 g/L, 1.35 g/L, 1.36 g/L, 1.37 g/L, 1.38 g/L, 1.39 g/L, 1.4 g/L, 1.41 g/L, 1.42 g/L, 1.43 g/L, 1.44 g/L, 1.45 g/L, 1.46 g/L, 1.47 g/L, 1.48 g/L, 1.49 g/L, 1.5 g/L, 1.51 g/L, 1.52 g/L, 1.53 g/L, 1.54 g/L, 1.55 g/L, 1.56 g/L, 1.57 g/L, 1.58 g/L, 1.59 g/L, 1.6 g/L, 1.61 g/L, 1.62 g/L, 1.63 g/L, 1.64 g/L, 1.65 g/L, 1.66 g/L, 1.67 g/L, 1.68 g/L, 1.69 g/L, 1.7 g/L, 1.71 g/L, 1.72 g/L, 1.73 g/L, 1.74 g/L, 1.75 g/L, 1.76 g/L, 1.77 g/L, 1.78 g/L, 1.79 g/L, 1.8 g/L, 1.81 g/L, 1.82 g/L, 1.83 g/L, 1.84 g/L, 1.85 g/L, 1.86 g/L, 1.87 g/L, 1.88 g/L, 1.89 g/L, 1.9 g/L, 1.91 g/L, 1.92 g/L, 1.93 g/L, 1.94 g/L, 1.95 g/L, 1.96 g/L, 1.97 g/L, 1.98 g/L, 1.99 g/L, 2 g/L, 2.01 g/L, 2.02 g/L, 2.03 g/L, 2.04 g/L, 2.05 g/L, 2.06 g/L, 2.07 g/L, 2.08 g/L, 2.09 g/L, 2.1 g/L, 2.11 g/L, 2.12 g/L, 2.13 g/L, 2.14 g/L, 2.15 g/L, 2.16 g/L, 2.17 g/L, 2.18 g/L, 2.19 g/L, 2.2 g/L, 2.21 g/L, 2.22 g/L, 2.23 g/L, 2.24 g/L, 2.25 g/L, 2.26 g/L, 2.27 g/L, 2.28 g/L, 2.29 g/L, 2.3 g/L, 2.31 g/L, 2.32 g/L, 2.33 g/L, 2.34 g/L, 2.35 g/L, 2.36 g/L, 2.37 g/L, 2.38 g/L, 2.39 g/L, 2.4 g/L, 2.41 g/L, 2.42 g/L, 2.43 g/L, 2.44 g/L, 2.45 g/L, 2.46 g/L, 2.47 g/L, 2.48 g/L, 2.49 g/L, 2.5 g/L, 2.51 g/L, 2.52 g/L, 2.53 g/L, 2.54 g/L, 2.55 g/L, 2.56 g/L, 2.57 g/L, 2.58 g/L, 2.59 g/L, 2.6 g/L, 2.61 g/L, 2.62 g/L, 2.63 g/L, 2.64 g/L, 2.65 g/L, 2.66 g/L, 2.67 g/L, 2.68 g/L, 2.69 g/L, 2.7 g/L, 2.71 g/L, 2.72 g/L, 2.73 g/L, 2.74 g/L, 2.75 g/L, 2.76 g/L, 2.77 g/L, 2.78 g/L, 2.79 g/L, 2.8 g/L, 2.81 g/L, 2.82 g/L, 2.83 g/L, 2.84 g/L, 2.85 g/L, 2.86 g/L, 2.87 g/L, 2.88 g/L, 2.89 g/L, 2.9 g/L, 2.91 g/L, 2.92 g/L, 2.93 g/L, 2.94 g/L, 2.95 g/L, 2.96 g/L, 2.97 g/L, 2.98 g/L, 2.99 g/L, or 3 g/L.

In another embodiment, other related products can be used, such as dried brewer's yeast (DBY) or spent brewers yeast, corn steep liquor or corn steep powder. When corn steep liquor is used, the usage rate would be approximately the same as for corn steep powder on a solids basis. In another embodiment, the corn steep powder (or solids or liquor) is added in relation to the amount of carbon substrate that is present or that will be added. When added in this way, beneficial amounts of corn steep powder (or liquor or solids) can include about 1:1 to about 1:6 g/g carbon, about 1:1 to about 1:5 g/g carbon, or about 1:2 to about 1:4 g/g carbon. In another embodiment, ratios as high as about 1.5:1 g/g carbon or about 3:1 g/g carbon or as low as about 1:8 g/g carbon or about 1:10 g/g carbon are used. In another embodiment, the ratio is 2:1 g/g carbon, 1.9:1 g/g carbon, 1.8:1 g/g carbon, 1.7:1 g/g carbon, 1.6:1 g/g carbon, 1.5:1 g/g carbon, 1.4:1 g/g carbon, 1.3:1 g/g carbon, 1.2:1 g/g carbon, 1.1:1 g/g carbon, 1:1 g/g carbon, 1:1.1 g/g carbon, 1:1.2 g/g carbon, 1:1.3 g/g carbon, 1:1.4 g/g carbon, 1:1.5 g/g carbon, 1:1.6 g/g carbon, 1:1.7 g/g carbon, 1:1.8 g/g carbon, 1:1.9 g/g carbon, 1:2 g/g carbon, 1:2.1 g/g carbon, 1:2.2 g/g carbon, 1:2.3 g/g carbon, 1:2.4 g/g carbon, 1:2.5 g/g carbon, 1:2.6 g/g carbon, 1:2.7 g/g carbon, 1:2.8 g/g carbon, 1:2.9 g/g carbon, 1:3 g/g carbon, 1:3.1 g/g carbon, 1:3.2 g/g carbon, 1:3.3 g/g carbon, 1:3.4 g/g carbon, 1:3.5 g/g carbon, 1:3.6 g/g carbon, 1:3.7 g/g carbon, 1:3.8 g/g carbon, 1:3.9 g/g carbon, 1:4 g/g carbon, 1:4.1 g/g carbon, 1:4.2 g/g carbon, 1:4.3 g/g carbon, 1:4.4 g/g carbon, 1:4.5 g/g carbon, 1:4.6 g/g carbon, 1:4.7 g/g carbon, 1:4.8 g/g carbon, 1:4.9 g/g carbon, 1:5 g/g carbon, 1:5.1 g/g carbon, 1:5.2 g/g carbon, 1:5.3 g/g carbon, 1:5.4 g/g carbon, 1:5.5 g/g carbon, 1:5.6 g/g carbon, 1:5.7 g/g carbon, 1:5.8 g/g carbon, 1:5.9 g/g carbon, 1:6 g/g carbon, 1:6.1 g/g carbon, 1:6.2 g/g carbon, 1:6.3 g/g carbon, 1:6.4 g/g carbon, 1:6.5 g/g carbon, 1:6.6 g/g carbon, 1:6.7 g/g carbon, 1:6.8 g/g carbon, 1:6.9 g/g carbon, 1:7 g/g carbon, 1:7.1 g/g carbon, 1:7.2 g/g carbon, 1:7.3 g/g carbon, 1:7.4 g/g carbon, 1:7.5 g/g carbon, 1:7.6 g/g carbon, 1:7.7 g/g carbon, 1:7.8 g/g carbon, 1:7.9 g/g carbon, 1:8 g/g carbon, 1:8.1 g/g carbon, 1:8.2 g/g carbon, 1:8.3 g/g carbon, 1:8.4 g/g carbon, 1:8.5 g/g carbon, 1:8.6 g/g carbon, 1:8.7 g/g carbon, 1:8.8 g/g carbon, 1:8.9 g/g carbon, 1:9 g/g carbon, 1:9.1 g/g carbon, 1:9.2 g/g carbon, 1:9.3 g/g carbon, 1:9.4 g/g carbon, 1:9.5 g/g carbon, 1:9.6 g/g carbon, 1:9.7 g/g carbon, 1:9.8 g/g carbon, 1:9.9 g/g carbon, or 1:10 g/g carbon.

In one embodiment, beneficial fermentation results can be achieved by adding corn steep powder in combination with DBY to the fermentation. The corn steep powder and DBY can also be fed throughout the course of the entire fermentation or a portion of the fermentation, continuously or delivered at intervals.

In one embodiment, the beneficial compounds from corn steep powder and/or yeast extract, such as glycine, histidine, isoleucine, proline, or phytate as well as combinations of these compounds can be added to the medium or broth to obtain a beneficial effect.

Various embodiments offer benefits relating to improving the titer and/or productivity of alcohol production by Q.17, Q.18, Q.19 or Q.20 by culturing the organism in a medium comprising one or more compounds comprising particular fatty acid moieties and/or culturing the organism under conditions of controlled pH.

In one embodiment, production of high levels of alcohol uses an organism with the ability to thrive in the presence of elevated alcohol levels and the ability to continue to produce alcohol without undue inhibition or suppression by the alcohol and/or other components present. Frequently, different metabolic pathways will be implicated for each of these. For example, pathways related to cell growth generally include those related to protein production, membrane production as well as the production of all of the cellular subsystems necessary for the cell to survive. Pathways related to alcohol production will frequently be more specific, such as those pathways related to the metabolism of sugars leading to production of alcohol and the enzymes that are necessary for the production of alcohol and intermediates. The pathway for one alcohol, e.g., ethanol, can share some similar enzymes, etc., but will also have enzymes and substrates specific to that pathway. While there can be some overlap between these sets of pathways, it is not expected that enhancement of one will automatically result in the enhancement of the other.

In some cases, alcohol intolerance or alcohol-induced toxicity can be related to permeabilization of the cell membrane by elevated levels of alcohol, leading to leakage of intracellular enzymes and nutrients. In some other cases, alcohol tolerance and the ability to produce high alcohol titers is related to the ability of intracellular enzymes to withstand denaturing by the alcohol present, e.g., within the cell, whether due to production by the cell itself or from transport across the cell membrane. In some cases, a more robust membrane will allow a higher alcohol gradient to be present across the membrane, thus allowing the cells to grow and/or continue to produce alcohol at higher external alcohol concentrations.

In one embodiment, Q.17, Q.18, Q.19 or Q.20 is fermented with a substrate at about pH 5-8.5. In one embodiment, a Q.17, Q.18, Q.19 or Q.20 is fermented at pH of about 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

### Acidic Culture Conditions

In another aspect, methods of producing alcohol; e.g., ethanol, comprising culturing Q.17, Q.18, Q.19 or Q.20 in a medium under conditions of controlled pH. In one embodiment, a culture of Q.17, Q.18, Q.19 or Q.20 can be grown at an acidic pH are provided herein. The medium that the culture is grown in can include a carbon Feedstock such as agricultural crops, algae, crop residues, modified crop plants, trees, wood chips, sawdust, paper, cardboard, or other materials containing cellulose, hemicellulosic, lignocellulose, pectin, polyglucose, polyfructose, and/or hydrolyzed forms of these. Additional nutrients can be present including sulfur- and nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyzed yeast, autolyzed yeast, dried brewer's yeast, corn steep liquor, corn steep solids, monosodium glutamate, and/or other fermentation nitrogen sources, vitamins, cofactors and/or mineral supplements. The Feedstock can be pretreated or not, such as described in U.S. patent application Ser. No. 12/919,750, filed Aug. 26, 2010 or PCT Application No. PCT/US10/40502, filed on Jun. 29, 2010, which are herein incorporated by reference in their entireties. The procedures and techniques for growing the organism to produce a fuel or other desirable chemical such as is described in incorporated U.S. patent application Ser. No. 12/720,574 which is herein incorporated by reference in its entirety.

In one embodiment, the pH of the medium is controlled at less than about pH 7.2 for at least a portion of the fermentation. In one embodiment, the pH is controlled within a range of about pH 3.0 to about 7.1 or about pH 4.5 to about 7.1, or about pH 5.0 to about 6.3, or about pH 5.5 to about 6.3, or about pH 6.0 to about 6.5, or about pH 5.5 to about 6.9 or about pH 6.2 to about 6.7. The pH can be controlled by the addition of a pH modifier. In one embodiment, a pH modifier is an acid, a base, a buffer, or a material that reacts with other materials present to serve to raise of lower the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. When more than one pH modifiers are utilized, they can be added at the same time or at different times. In one embodiment, one or more acids and one or more bases can be combined, resulting in a buffer. In one embodiment, media components, such as a carbon source or a nitrogen source can also serve as a pH modifier; suitable media components include those with high or low pH or those with buffering capacity. Exemplary media components include acid- or base-hydrolyzed plant polysaccharides having with residual acid or base, AFEX treated plant material with residual ammonia, lactic acid, corn steep solids or liquor.

In one embodiment, the pH modifier can be added as a part of the medium components prior to inoculation with Q.17, Q.18, Q.19 or Q.20. In one embodiment, the pH modifier can also be added after inoculation with the Q.17, Q.18, Q.19 or Q.20. In one embodiment, sufficient buffer capacity can be added to the seed fermentation by way of various pH modifiers and/or other medium components and/or metabolites to provide adequate pH control during the final fermentation stage. In one embodiment, a pH modifier is added only to the final fermentation stage. In one embodiment, pH modifier is added to both the seed stage and the final stage. In one embodiment, the pH is monitored throughout the fermentation and is adjusted in response to changes in the fermentation. In one embodiment, the pH modifier is added whenever the pH of the fermentation changes by a pH value of about 0.005, 0.01, 0.05, 0.1, 0.2, 0.3, 0.4, 0.5 or more at any stage of the fermentation. In one embodiment, the pH modifier is added whenever the alcohol content of the fermentation is about 0.5 g/L, 1.0 g/L, 2.0 g/L, or 5.0 g/L or more. In one embodiment, different types of pH modifiers are utilized at different stages or points in the fermentation, such as a buffer being used at the seed stage, and base and/or acid added in the final fermenter, or an acid being used at one time and a base at another time.

In one embodiment, a constant pH can be utilized throughout the fermentation. In one embodiment, the timing and/or amount of pH reduction can be related to the growth conditions of the cells, such as in relation to the cell count, the alcohol produced, the alcohol present, or the rate of alcohol production. In one embodiment, the pH reduction can be made in relation to physical or chemical properties of the fermentation, such as viscosity, medium composition, gas production, off gas composition, etc.

Non-limiting examples of suitable buffers include salts of phosphoric acid, including monobasic, dibasic, and tribasic salts, mixtures of these salts and mixtures with the acid; salts of citric acid, including the various basic forms, mixtures and mixtures with the acid; and salts of carbonate.

Suitable acids and bases that can be used as pH modifiers include any liquid or gaseous acid or base that is compatible with the organism. Examples include ammonia, ammonium hydroxide, sulfuric acid, lactic acid, citric acid, phosphoric acid, sodium hydroxide, and HCl. In some cases, the selection of the acid or base can be influenced by the compatibility of the acid or base with equipment being used for fermentation. In some cases, both an acid addition, to lower pH or consume base, and a base addition, to raise pH or consume acid, can be used in the same fermentation.

The timing and amount of pH modifier to add can be determined from a measurement of the pH of the contents of the fermentor, such as by grab sample or by a submerged pH probe, or it can be determined based on other parameters such as the time into the fermentation, gas generation, viscosity, alcohol production, titration, etc. In one embodiment, a combination of these techniques can be used.

In one embodiment, the pH of the fermentation is initiated at a neutral pH and then is reduced to an acidic pH when the production of alcohol is detected. In another embodiment, the pH of the fermentation is initiated at an acidic pH and is maintained at an acidic pH until the fermentation reaches a stationary phase of growth.

### Fatty Acid Medium Component and Acidic Culture Conditions

In another embodiment, a combination of adding a fatty acid comprising compound to the medium and fermenting at reduced pH can be used. In one embodiment, addition of a fatty acid, such as a free fatty acid fulfills both techniques: adding a fatty acid compound and lowering the pH of the fermentation. In one embodiment, different compounds can be added to accomplish each technique. For example, a vegetable oil can be added to the medium to supply the fatty acid and then a mineral acid or an organic acid can be added during the fermentation to reduce the pH to a suitable level, as described above. When the fermentation includes both operation at reduced pH and addition of fatty acid comprising compounds, the methods and techniques described herein for each type of operation separately can be used together. In one embodiment, the operation at low pH and the presence of the fatty acid comprising compounds will be at the same time. In one embodiment, the presence of fatty acid comprising compounds will precede operation at low pH, and in one embodiment, operation at low pH will precede the addition of fatty acid comprising compounds. In one embodiment, the operation at low pH and the presence of the fatty acid will be prior to inoculation with Q.17, Q.18, Q.19 or Q.20. In one embodiment, the operation at low pH will be prior to inoculation with Q.17, Q.18, Q.19 or Q.20 and the presence of the fatty acid will occur after or during inoculation with Q.17, Q.18, Q.19 or Q.20. In one embodiment, the presence of the fatty acid will be prior to inoculation with Q.17, Q.18, Q.19 or Q.20 and the operation at low pH will occur after or during the inoculation with Q.17, Q.18, Q.19 or Q.20. In one embodiment, the operation at low pH and the presence of the fatty acid will be after inoculation with Q.17, Q.18, Q.19 or Q.20. In one embodiment, the operation at low pH and the presence of the fatty acid will be at other stages of fermentation.

### Genetic Modification of Q.17, Q.18, Q.19 or Q.20

In another aspect, compositions and methods to produce a fermentation end-product, such as a fuel, such as one or more alcohols, e.g., ethanol, by the creation and use of a genetically modified Q.17, Q.18, Q.19 or Q.20 are provided. In one embodiment, regulating fermentative biochemical pathways, over expression of saccharolytic enzymes, or increasing tolerance of environmental conditions during fermentation of Q.17, Q.18, Q.19 or Q.20 is provided. One example of methods that can be used to enhance expression of saccharolytic enzymes can be found in U.S. patent application Ser. No. 12/630,784 filed Dec. 3, 2009. In one embodiment, modification of other strains of C. phytofermentans to uncouple regulation of cellulases or other enzymes is provided. In one embodiment, Q.17, Q.18, Q.19 or Q.20 is transformed with heterologous polynucleotides encoding one or more genes for the pathway, enzyme, or protein of interest. In another embodiment, Q.17, Q.18, Q.19 or Q.20 is transformed to produce multiple copies of one or more genes for the pathway, enzyme, or protein of interest. In one embodiment, Q.17, Q.18, Q.19 or Q.20 is transformed with heterologous polynucleotides encoding one or more genes encoding enzymes for the hydrolysis and/or fermentation of a hexose, wherein said genes are expressed at sufficient levels to confer upon said Q.17, Q.18, Q.19 or Q.20 transformant the ability to produce ethanol at increased concentrations, productivity levels or yields compared to Q.17, Q.18, Q.19 or Q.20 that is not transformed. In such ways, an enhanced rate of ethanol production can be achieved.

In another embodiment, Q.17, Q.18, Q.19 or Q.20 is transformed with heterologous polynucleotides encoding one or more genes encoding saccharolytic enzymes for the saccharification of a polysaccharide, wherein said genes are expressed at sufficient levels to confer upon said Q.17, Q.18, Q.19 or Q.20 transformant the ability to saccharify a polysaccharide to mono-, di- or oligosaccharides at further increased concentrations, rates of saccharification or yields of mono-, di- or oligosaccharides compared to Q.17, Q.18, Q.19 or Q.20 that is not transformed. The production of a saccharolytic enzyme by the host, and the subsequent release of that saccharolytic enzyme into the medium, can reduce the amount of commercial enzyme used to degrade biomass or polysaccharides into fermentable monosaccharides and oligosaccharides. The saccharolytic DNA can be native to the host, although more often the DNA will be foreign, and heterologous. Advantageous saccharolytic genes include cellulolytic, xylanolytic, and starch-degrading enzymes such as cellulases, xylanases, and amylases. The saccharolytic enzymes can be at least partially secreted by the host, or it can be accumulated substantially intracellularly for subsequent release. Advantageously, intracellularly-accumulated enzymes which are thermostable, can be released when desired by heat-induced lysis. Combinations of enzymes can be encoded by the heterologous DNA, some of which are secreted, and some of which are accumulated.

Other modifications can be made to enhance the ethanol production of the recombinant bacteria. For example, the host can further comprise an additional heterologous DNA segment, the expression product of which is a protein involved in the transport of mono- and/or oligosaccharides into the recombinant host. Likewise, additional genes from the glycolytic pathway can be incorporated into the host to redirect the bioenergetics of the ethanolic production pathways. In such ways, an enhanced rate of ethanol production can be achieved.

In order to improve the production of biofuels (e.g. ethanol), modifications can be made in transcriptional regulators, genes for the formation of organic acids, carbohydrate transporter genes, sporulation genes, genes that influence the formation/regenerate of enzymatic cofactors, genes that further influence ethanol tolerance, genes that influence salt tolerance, genes that influence growth rate, genes that influence oxygen tolerance, genes that influence catabolite repression, genes that influence hydrogen production, genes that influence resistance to heavy metals, genes that influence resistance to acids or genes that influence resistance to aldehydes.

Those skilled in the art will appreciate that a number of modifications can be made to the methods exemplified herein. For example, a variety of promoters can be utilized to drive expression of the heterologous genes in the recombinant Clostridium sp. host. The skilled artisan, having the benefit of the instant disclosure, will be able to readily choose and utilize any one of the various promoters available for this purpose. Similarly, skilled artisans, as a matter of routine preference, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target organism. This DNA can be ligated to form circles without functional or with conditionally functional replicons and used for transformation. Thus, the gene of interest can be introduced in a heterologous host such as E. coli, and short, random fragments can be isolated and ligated in Clostridium sp. to promote homologous recombination.

In one embodiment, a microorganism can be genetically modified to enhance enzyme activity of one or more enzymes, including but not limited to hydrolytic enzymes (such as cellulase(s), hemicellulase(s), or pectinase(s) etc.). In one embodiment, a method is used to genetically modify a microorganism (such as a Clostridium species) that is disclosed in US 20100086981 or PCT/US2010/40494, which are herein incorporated by reference in their entirety. In another embodiment, an enzyme can be selected from the annotated genome of C. phytofermentans, another bacterial species, such as B. subtilis, E. coli, various Clostridium species, or yeasts such as S. cerevisiae for utilization in products and processes described herein. Examples include enzymes such as L-butanediol dehydrogenase, acetoin reductase, 3-hydroxyacyl-CoA dehydrogenase, cis-aconitate decarboxylase or the like, to create pathways for new products from biomass.

Examples of such modifications include modifying endogenous nucleic acid regulatory elements to increase expression of one or more enzymes (e.g., operably linking a gene encoding a target enzyme to a strong promoter), introducing into a microorganism additional copies of endogenous nucleic acid molecules to provide enhanced activity of an enzyme by increasing its production, and operably linking genes encoding one or more enzymes to an inducible promoter or a combination thereof.

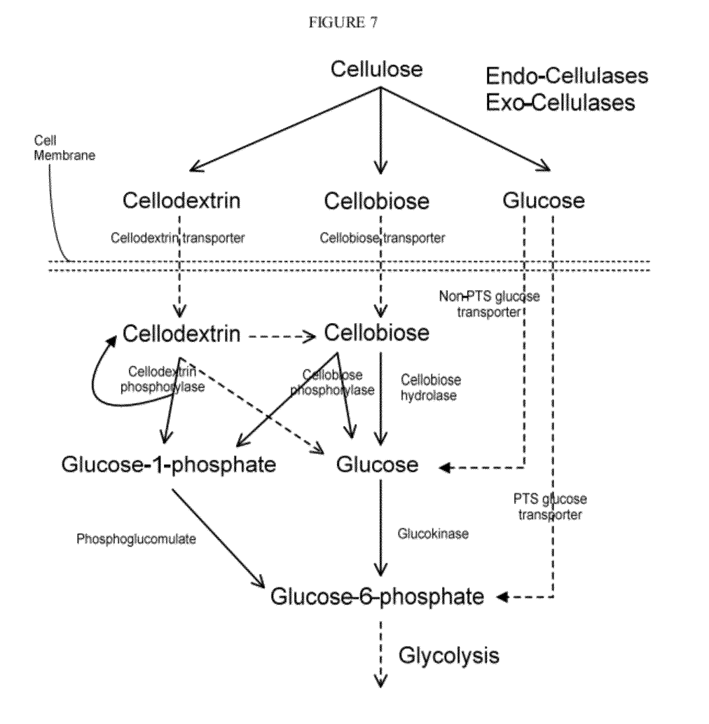

In another embodiment, a microorganism can be modified to enhance an activity of one or more hydrolytic enzymes (such as cellulase(s), hemicellulase(s), or pectinases etc.) or antioxidants (such as catalase), or other enzymes associated with cellulose processing. For example, in the case of cellulases, various microorganisms disclosed herein can be modified to enhance activity of one or more cellulases, or enzymes associated with cellulose processing (e.g., FIG. 7).

In one embodiment, a hydrolytic enzyme is selected from the annotated genome of C. phytofermentans for utilization in a product or process disclosed herein. In another embodiment, the hydrolytic enzyme is an endoglucanase, chitinase, cellobiohydrolase or endo-processive cellulases (either on reducing or non-reducing end).

In one embodiment, a microorganism, such as C. phytofermentans, can be modified to enhance production of one or more hydrolases. In another embodiment, one or more enzymes can be heterologous expressed in a host (e.g., a bacteria or yeast). For heterologous expression bacteria or yeast can be modified through recombinant technology. (e.g., Brat et al. Appl. Env. Microbio. 2009; 75(8):2304-2311, disclosing expression of xylose isomerase in S. cerevisiae and which is herein incorporated by reference in its entirety).

In another embodiment, other modifications can be made to enhance end-product (e.g., ethanol) production in a recombinant microorganism. For example, the host microorganism can further comprise an additional heterologous DNA segment, the expression product of which is a protein involved in the transport of mono- and/or oligosaccharides into the recombinant host. Likewise, additional genes from the glycolytic pathway can be incorporated into the host. In such ways, an enhanced rate of ethanol production can be achieved.

A variety of promoters (e.g., constitutive promoters, inducible promoters) can be used to drive expression of the heterologous genes in a recombinant host microorganism.

Promoter elements can be selected and mobilized in a vector (e.g., pIMPCphy). For example, a transcription regulatory sequence is operably linked to gene(s) of interest (e.g., in a expression construct). The promoter can be any array of DNA sequences that interact specifically with cellular transcription factors to regulate transcription of the downstream gene. The selection of a particular promoter depends on what cell type is to be used to express the protein of interest. In one embodiment, a transcription regulatory sequences can be derived from the host microorganism. In various embodiments, constitutive or inducible promoters are selected for use in a host cell. Depending on the host cell, there are potentially hundreds of constitutive and inducible promoters which are known and that can be engineered to function in the host cell.

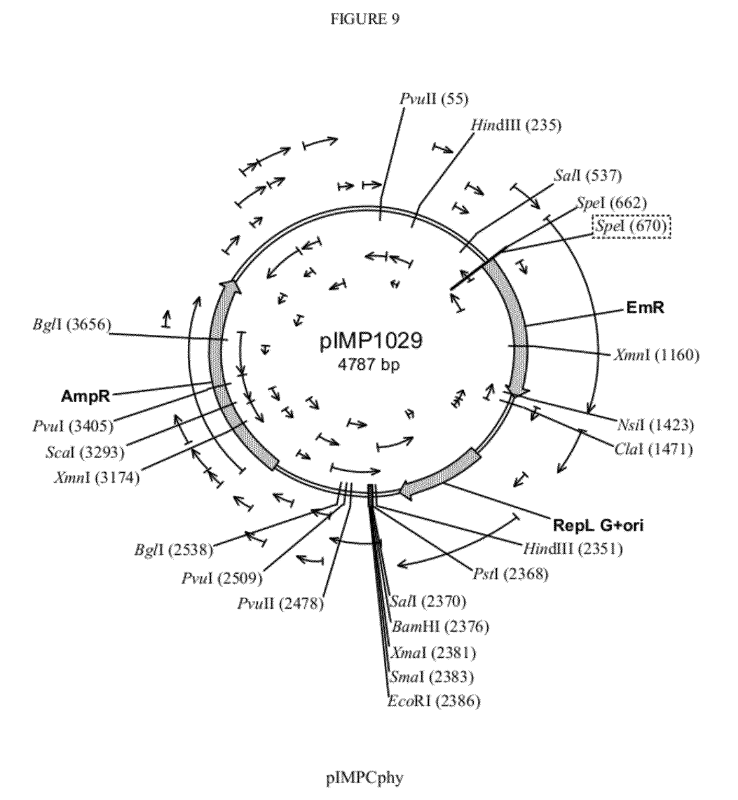

A map of the plasmid pIMPCphy is shown in FIG. 9, and the DNA sequence of this plasmid is provided as SEQ ID NO:1.

TABLE 2
Nucleotide sequence of plasmid pIMPCphy

SEQ ID NO: 1
gcgcccaatacgcaaaccgcctctccccgcgcgttggccgattcattaatgcagctggcacgacaggtttcccgactgga
aagcgggcagtgagcgcaacgcaattaatgtgagttagctcactcattaggcaccccaggctttacactttatgcttccg
gctcgtatgttgtgtggaattgtgagcggataacaatttcacacaggaaacagctatgaccatgattacgccaaagcttt
ggctaacacacacgccattccaaccaatagttttctcggcataaagccatgctctgacgcttaaatgcactaatgcctta
aaaaaacattaaagtctaacacactagacttatttacttcgtaattaagtcgttaaaccgtgtgctctacgaccaaaagt
ataaaacctttaagaactttcttttttcttgtaaaaaaagaaactagataaatctctcatatcttttattcaataatcgc
atcagattgcagtataaatttaacgatcactcatcatgttcatatttatcagagctccttatattttatttcgatttatt
tgttatttatttaacatttttctattgacctcatcttttctatgtgttattcttttgttaattgtttacaaataatctac
gatacatagaaggaggaaaaactagtatactagtatgaacgagaaaaatataaaacacagtcaaaactttattacttcaa
aacataatatagataaaataatgacaaatataagattaaatgaacatgataatatctttgaaatcggctcaggaaaaggg
cattttacccttgaattagtacagaggtgtaatttcgtaactgccattgaaatagaccataaattatgcaaaactacaga
aaataaacttgttgatcacgataatttccaagttttaaacaaggatatattgcagtttaaatttcctaaaaaccaatcct
ataaaatatttggtaatataccttataacataagtacggatataatacgcaaaattgtttttgatagtatagctgatgag
atttatttaatcgtggaatacgggtttgctaaaagattattaaatacaaaacgctcattggcattatttttaatggcaga
agttgatatttctatattaagtatggttccaagagaatattttcatcctaaacctaaagtgaatagctcacttatcagat
taaatagaaaaaaatcaagaatatcacacaaagataaacagaagtataattatttcgttatgaaatgggttaacaaagaa
tacaagaaaatatttacaaaaaatcaatttaacaattccttaaaacatgcaggaattgacgatttaaacaatattagctt
tgaacaattcttatctcttttcaatagctataaattatttaataagtaagttaagggatgcataaactgcatcccttaac
ttgtttttcgtgtacctattttttgtgaatcgatccggccagcctcgcagagcaggattcccgttgagcaccgccaggtg
cgaataagggacagtgaagaaggaacacccgctcgcgggtgggcctacttcacctatcctgcccggatcgattatgtctt
ttgcgcattcacttcttttctatataaatatgagcgaagcgaataagcgtcggaaaagcagcaaaaagtttcctttttgc
tgttggagcatgggggttcagggggtgcagtatctgacgtcaatgccgagcgaaagcgagccgaagggtagcatttacgt
tagataaccccctgatatgctccgacgctttatatagaaaagaagattcaactaggtaaaatcttaatataggttgagat
gataaggtttataaggaatttgtttgttctaatttttcactcattttgttctaatttcttttaacaaatgttcttttttt
tttagaacagttatgatatagttagaatagtttaaaataaggagtgagaaaaagatgaaagaaagatatggaacagtcta
taaaggctctcagaggctcatagacgaagaaagtggagaagtcatagaggtagacaagttataccgtaaacaaacgtctg
gtaacttcgtaaaggcatatatagtgcaattaataagtatgttagatatgattggcggaaaaaaacttaaaatcgttaac
tatatcctagataatgtccacttaagtaacaatacaatgatagctacaacaagagaaatagcaaaagctacaggaacaag
tctacaaacagtaataacaacacttaaaatcttagaagaaggaaatattataaaaagaaaaactggagtattaatgttaa
accctgaactactaatgagaggcgacgaccaaaaacaaaaatacctcttactcgaatttgggaactttgagcaagaggca
aatgaaatagattgacctcccaataacaccacgtagttattgggaggtcaatctatgaaatgcgattaagcttagcttgg
ctgcaggtcgacggatccccgggaattcactggccgtcgttttacaacgtcgtgactgggaaaaccctggcgttacccaa
cttaatcgccttgcagcacatccccctttcgccagctggcgtaatagcgaagaggcccgcaccgatcgcccttcccaaca
gttgcgcagcctgaatggcgaatggcgcctgatgcggtattttctccttacgcatctgtgcggtatttcacaccgcatat
ggtgcactctcagtacaatctgctctgatgccgcatagttaagccagccccgacacccgccaacacccgctgacgcgccc
tgacgggcttgtctgctcccggcatccgcttacagacaagctgtgaccgtctccgggagctgcatgtgtcagaggttttc
accgtcatcaccgaaacgcgcgagacgaaagggcctcgtgatacgcctatttttataggttaatgtcatgataataatgg
tttcttagacgtcaggtggcacttttcggggaaatgtgcgcggaacccctatttgtttatttttctaaatacattcaaat
atgtatccgctcatgagacaataaccctgataaatgcttcaataatattgaaaaaggaagagtatgagtattcaacattt
ccgtgtcgcccttattcccttttttgcggcattttgccttcctgtttttgctcacccagaaacgctggtgaaagtaaaag
atgctgaagatcagttgggtgcacgagtgggttacatcgaactggatctcaacagcggtaagatccttgagagttttcgc
cccgaagaacgttttccaatgatgagcacttttaaagttctgctatgtggcgcggtattatcccgtattgacgccgggca
agagcaactcggtcgccgcatacactattctcagaatgacttggttgagtactcaccagtcacagaaaagcatcttacgg
atggcatgacagtaagagaattatgcagtgctgccataaccatgagtgataacactgcggccaacttacttctgacaacg
atcggaggaccgaaggagctaaccgcttttttgcacaacatgggggatcatgtaactcgccttgatcgttgggaaccgga
gctgaatgaagccataccaaacgacgagcgtgacaccacgatgcctgtagcaatggcaacaacgttgcgcaaactattaa
ctggcgaactacttactctagcttcccggcaacaattaatagactggatggaggcggataaagttgcaggaccacttctg
cgctcggcccttccggctggctggtttattgctgataaatctggagccggtgagcgtgggtctcgcggtatcattgcagc
actggggccagatggtaagccctcccgtatcgtagttatctacacgacggggagtcaggcaactatggatgaacgaaata
gacagatcgctgagataggtgcctcactgattaagcattggtaactgtcagaccaagtttactcatatatactttagatt
gatttaaaacttcatttttaatttaaaaggatctaggtgaagatcctttttgataatctcatgaccaaaatcccttaacg
tgagttttcgttccactgagcgtcagaccccgtagaaaagatcaaaggatcttcttgagatcctttttttctgcgcgtaa
tctgctgcttgcaaacaaaaaaaccaccgctaccagcggtggtttgtttgccggatcaagagctaccaactctttttccg
aaggtaactggcttcagcagagcgcagataccaaatactgtccttctagtgtagccgtagttaggccaccacttcaagaa
ctctgtagcaccgcctacatacctcgctctgctaatcctgttaccagtggctgctgccagtggcgataagtcgtgtctta
ccgggttggactcaagacgatagttaccggataaggcgcagcggtcgggctgaacggggggttcgtgcacacagcccagc
ttggagcgaacgacctacaccgaactgagatacctacagcgtgagctatgagaaagcgccacgcttcccgaagggagaaa
ggcggacaggtatccggtaagcggcagggtcggaacaggagagcgcacgagggagcttccagggggaaacgcctggtatc
tttatagtcctgtcgggtttcgccacctctgacttgagcgtcgatttttgtgatgctcgtcaggggggcggagcctatgg
aaaaacgccagcaacgcggcctttttacggttcctggccttttgctggccttttgctcacatgttctttcctgcgttatc
ccctgattctgtggataaccgtattaccgcctttgagtgagctgataccgctcgccgcagccgaacgccgagcgcagcga
gtcagtgagcgaggaagcggaaga

The vector pIMPCphy was constructed as a shuttle vector for C. phytofermentans and is further described in U.S. Patent Application Publication US20100086981, which is herein incorporated by reference in its entirety. It has an Ampicillin-resistance cassette and an Origin of Replication (ori) for selection and replication in E. coli. It contains a Gram-positive origin of replication that can enable the replication of the plasmid in C. phytofermentans. In order to select for the presence of the plasmid, the pIMPCphy carries an erythromycin resistance gene under the control of the C. phytofermentans promoter of the gene Cphy1029. This plasmid can be transferred to C. phytofermentans by electroporation or by transconjugation with an E. coli strain that has a mobilizing plasmid, for example pRK2030. A plasmid map of pIMPCphy is depicted in FIG. 9. pIMPCphy is an effective replicative vector system for all microorganisms, including all gram⁺and gram-bacteria, and fungi (including yeasts). A further discussion of promoters, regulation of gene expression products, and additional genetic modifications can be found in U.S. Patent Application Publication US 20100086981A1, which is herein incorporated by reference in its entirety.

Non-Recombinant Genetic Modification

In other embodiments, a microorganism can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenized microorganisms can then be screened for beneficial mutations that exhibit one or more desirable properties. Screening can be performed by growing the mutagenized microorganisms on substrates that comprise carbon sources that will be utilized during the generation of end-products by fermentation. Screening can also include measuring the production of end-products during growth of the microorganism, or measuring the digestion or assimilation of the carbon source(s). The isolates so obtained can further be transformed with recombinant polynucleotides or used in combination with any of the methods and compositions provided herein to further enhance biofuel production.

Various methods can be used to produce and select mutants that differ from wild-type cells. In some instances, bacterial populations are treated with a mutagenic agent, for example, nitrosoguanidine (N-methyl-N′-nitro-N-nitrosoguanidine) or the like, to increase the mutation frequency above that of spontaneous mutagenesis. This is induced mutagenesis. Techniques for inducing mutagenesis include, but are not limited to, exposure of the bacteria to a mutagenic agent, such as x-rays or chemical mutagenic agents. More sophisticated procedures involve isolating the gene of interest and making a change in the desired location, then reinserting the gene into bacterial cells. This is site-directed mutagenesis.

Directed evolution is usually performed as three steps which can be repeated more than once. First, the gene encoding a protein of interest is mutated and/or recombined at random to create a large library of gene variants. The library is then screened or selected for the presence of mutants or variants that show the desired property. Screens enable the identification and isolation of high-performing mutants by hand; selections automatically eliminate all non functional mutants. Then the variants identified in the selection or screen are replicated, enabling DNA sequencing to determine what mutations occurred. Directed evolution can be carried out in vivo or in vitro. See, for example, Otten, L. G.; Quax, W. J. (2005). Biomolecular Engineering 22 (1-3): 1-9; Yuan, L., et al. (2005) Microbiol. Mol. Biol. Rev. 69 (3): 373-392.

### Fermentation Plant and Process of Producing Fermentation End-Products

Large Scale Fermentation End-Product Production from Biomass

Generally, there are two basic approaches to producing fermentation end-products (e.g., fuel grade ethanol) from biomass on a large scale utilizing microbial cells (e.g., Q.17, Q.18, Q.19 or Q.20 cells). In the first method, one first hydrolyzes a biomass material that includes high molecular weight carbohydrates to lower molecular weight carbohydrates, and then ferments the lower molecular weight carbohydrates utilizing of microbial cells to produce ethanol. In the second method, one ferments the biomass material itself without chemical and/or enzymatic pretreatment. In the first method, hydrolysis can be accomplished using acids, e.g., Bronsted acids (e.g., sulfuric or hydrochloric acid), bases, e.g., sodium hydroxide, hydrothermal processes, ammonia fiber explosion processes (“AFEX”), lime processes, enzymes, or combination of these. Hydrogen, and other products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the biocatalyst cells, which can increase fermentation rate and yield. Removal of lignin can, e.g., provide a combustible fuel for driving a boiler, and can also, e.g., increase porosity and/or surface area of the biomass, often increasing fermentation rate and yield. Generally, in any of the below described embodiments, the initial concentration of the carbohydrates in the medium can be greater than 20 mM, e.g., greater than 30 mM, 50 mM, 75 mM, 100 mM, 150 mM, 200 mM, or even greater than 500 mM.

Biomass Processing Plant and Process of Producing Fermentation End-Products from Biomass

In one aspect, a fuel plant that includes a hydrolysis unit configured to hydrolyze a biomass material that includes a high molecular weight carbohydrate, a fermentor configured to house a medium with Q.17, Q.18, Q.19 or Q.20 cells or another C5/C6 hydrolyzing organism dispersed therein, and one or more product recovery system(s) to isolate a product or products and associated by-products and co-products is provided.

In another aspect, methods of making a product or products that include combining Q.17, Q.18, Q.19 or Q.20 cells or another C5/C6 hydrolyzing organism and a biomass feed in a medium, and fermenting the biomass material under conditions and for a time sufficient to produce a biofuel, chemical product or fermentation end-products, e.g. ethanol, propanol, hydrogen, lignin, terpenoids, and the like as described above, is provided.

In another aspect, products made by any of the processes described herein is also provided herein.

Fermentation End product Production From Biomass with Pretreatment

Generally, there are two basic approaches to producing chemical products from biomass on a large scale utilizing microorganisms such as Q.17, Q.18, Q.19 or Q.20 or other C5/C6 hydrolyzing organisms. In all methods, depending on the type of biomass and its physical manifestation, one of the processes can comprise a milling of the carbonaceous material, via wet or dry milling, to reduce the material in size and increase the surface to volume ratio (physical modification).

In a first method, one first hydrolyzes a biomass material that includes high molecular weight carbohydrates to delignify it or to separate the carbohydrate compounds from noncarbohydrate compounds. Using any combination of heat, chemical, and/or enzymatic treatment, the hydrolyzed material can be separated to form liquid and dewatered streams, which may or may not be separately treated and kept separate or recombined, and then ferments the lower molecular weight carbohydrates utilizing Q.17, Q.18, Q.19 or Q.20 cells or another C5/C6 hydrolyzing biocatalyst to produce one or more chemical products. In the second method, one ferments the biomass material itself without heat, chemical, and/or enzymatic pretreatment. In the first method, hydrolysis can be accomplished using acids (e.g. sulfuric or hydrochloric acids), bases (e.g. sodium hydroxide), hydrothermal processes, ammonia fiber explosion processes (“AFEX”), lime processes, enzymes, or combination of these. Hydrolysis and/or steam treatment of the biomass can, e.g., increase porosity and/or surface area of the biomass, often leaving the cellulosic materials more exposed to any C5/C6 hydrolyzing organism, such as Q.17, Q.18, Q.19 or Q.20 or C. phytofermentans, which can increase fermentation rate and yield. Hydrolysis and/or steam treatment of the biomass can, e.g., produce by-products or co-products which can be separated or treated to improve fermentation rate and yield, or used to produce power to run the process, or used as products with or without further processing. Removal of lignin can, e.g., provide a combustible fuel for driving a boiler. Gaseous, e.g., hydrogen and CO₂, liquid, e.g. ethanol and organic acids, and solid, e.g. lignin, products of the fermentation can be captured and purified if desired, or disposed of, e.g., by burning. For example, the hydrogen gas can be flared, or used as an energy source in the process, e.g., to drive a steam boiler, e.g., by burning. Products exiting the fermentor can be further processed, e.g. ethanol may be transferred to distillation and rectification, producing a concentrated ethanol mixture or solids may be separated for use to provide energy or as chemical products. It is understood that other methods of producing fermentation end products or biofuels can incorporate any and all of the processes described as well as additional or substitute processes that may be developed to economically or mechanically streamline these methods, all of which are meant to be incorporated in their entirety within the scope of this disclosure.

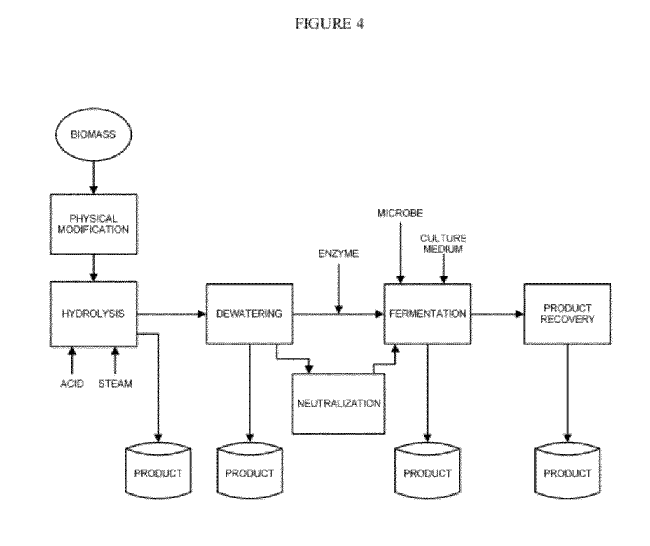

FIG. 4 is an example of a method for producing chemical products from biomass by first treating biomass with an acid at elevated temperature and pressure in a hydrolysis unit. The biomass may first be heated by addition of hot water or steam. The biomass may be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure may be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The hydrolysate is then discharged into the flash tank portion of the pretreatment unit, and is held in the tank for a period of time to further hydrolyze the biomass, e.g., into oligosaccharides and monomeric sugars, preferably oligomers. Steam explosion may also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 15% and 60%.

After pretreatment, the biomass may be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products may be derived from treatment of the acidified fluid, e.g., gypsum or ammonium phosphate. Enzymes or a mixture of enzymes can be added during pretreatment to assist, e.g. endoglucanases, exoglucanases, cellobiohydrolases (CBH), beta-glucosidases, glycoside hydrolases, glycosyltransferases, lyases, and esterases active against components of cellulose, hemicelluloses, pectin, and starch, in the hydrolysis of high molecular weight components.

The fermentor is fed with hydrolyzed biomass, any liquid fraction from biomass pretreatment, an active seed culture of Clostridium sp. cells, if desired a co-fermenting microorganism, e.g., yeast or E. coli, and, if required, nutrients to promote growth of Clostridium sp. or other microorganisms. Alternatively, the pretreated biomass or liquid fraction can be split into multiple fermentors, each containing a different strain of Clostridium sp. and/or other microorganisms, and each operating under specific physical conditions. Fermentation is allowed to proceed for a period of time, e.g., between about 15 and 150 hours, while maintaining a temperature of, e.g., between about 25° C. and 50° C. Gas produced during the fermentation is swept from fermentor and is discharged, collected, or flared with or without additional processing, e.g. hydrogen gas may be collected and used as a power source or purified as a co-product.

After fermentation, the contents of the fermentor are transferred to product recovery. Products are extracted, e.g., ethanol is recovered through distilled and rectification.

Fermentation End-Product Production From Biomass without Pretreatment

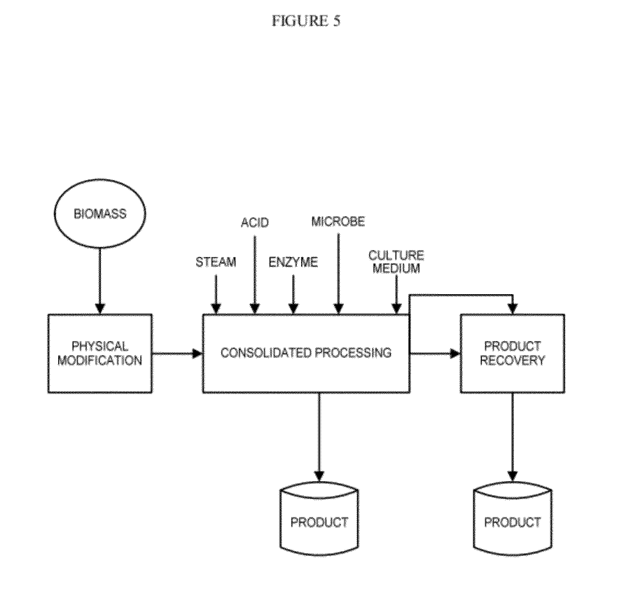

FIG. 5 depicts a method for producing chemicals from biomass by charging biomass to a fermentation vessel. The biomass may be allowed to soak for a period of time, with or without addition of heat, water, enzymes, or acid/alkali. The pressure in the processing vessel may be maintained at or above atmospheric pressure. Acid or alkali may be added at the end of the pretreatment period for neutralization. At the end of the pretreatment period, or at the same time as pretreatment begins, an active seed culture of Clostridium sp. cells or another C5/C6 hydrolyzing organism and, if desired, a co-fermenting microorganism, e.g., yeast or E. coli, and, if required, nutrients to promote growth of Clostridium sp. or other microorganisms are added. Fermentation is allowed to proceed as described above. After fermentation, the contents of the fermentor are transferred to product recovery as described above.

Any combination of the chemical production methods and/or features can be utilized to make a hybrid production method. In any of the methods described herein, products may be removed, added, or combined at any step. Clostridium sp. can be used alone, or synergistically in combination with one or more other microorganisms (e.g. yeasts, fungi, or other bacteria). Different methods can be used within a single plant to produce different products.

In another aspect, a fuel plant that includes a hydrolysis unit configured to hydrolyze a biomass material that includes a high molecular weight carbohydrate, and a fermentor configured to house a medium and contains Clostridium cells dispersed therein, is provided.

In another aspect, methods of making a fuel or fuels that include combining Clostridium cells and a lignocellulosic material (and/or other biomass material) in a medium, and fermenting the lignocellulosic material under conditions and for a time sufficient to produce a fuel or fuels, e.g., ethanol, propanol and/or hydrogen or another chemical compound is provided herein.

In one embodiment, a process for producing ethanol and hydrogen from biomass using acid hydrolysis pretreatment is provided. In one embodiment, a process for producing ethanol and hydrogen from biomass using enzymatic hydrolysis pretreatment is provided. In one embodiment, the process for producing ethanol and hydrogen from biomass is by using biomass that has not been enzymatically pretreated. Still in another embodiment, the process for producing ethanol and hydrogen from biomass is by using biomass that has not been chemically or enzymatically pretreated, but is optionally steam treated.

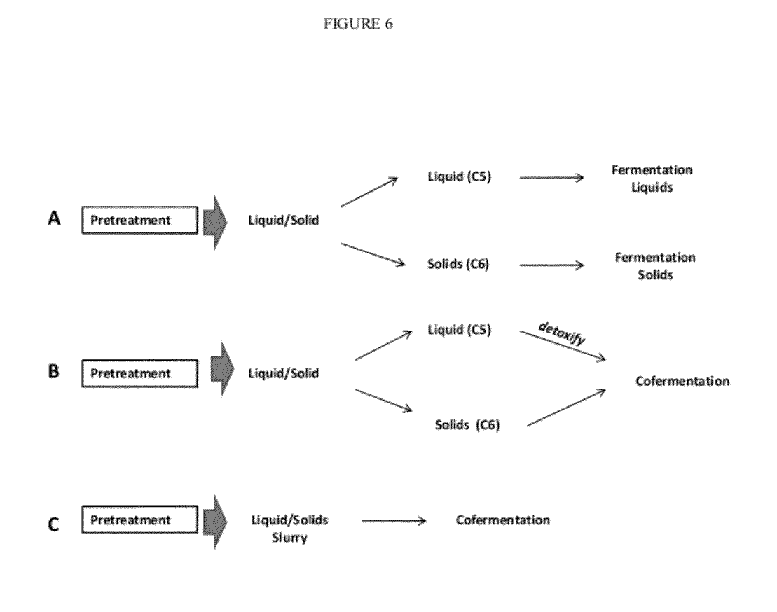

FIG. 6 discloses pretreatments that produce hexose or pentose saccharides or oligomers that are then unprocessed or processed further and either, fermented separately or together. FIG. 6A depicts a process (e.g., acid pretreatment) that produces a solids phase and a liquid phase which are then fermented separately. FIG. 6B depicts a similar pretreatment that produces a solids phase and liquids phase. The liquids phase is separated from the solids and elements that are toxic to the fermenting microorganism are removed prior to fermentation. At initiation of fermentation, the two phases are recombined and cofermented together. This is a more cost-effective process than fermenting the phases separately. The third process (FIG. 6C) is the least costly. The pretreatment results in a slurry of liquids or solids that are then cofermented together. There is little loss of saccharides component and minimal equipment required.

In another aspect, the products made by any of the processes described herein are provided.

### EXAMPLES

The following examples serve to illustrate certain embodiments and aspects and are not to be construed as limiting the scope thereof.

### Example 1

### Isolation of De-repressed Cellulase Mutant Strains

Cellulase deregulation mutants of Clostridium phytofermentans were screened by selecting mutants that could grow with phosphoric acid-swollen cellulose (PASC) as the sole carbon source in the presence of 2-deoxyglucose (DoG). DoG acts as a glucose mimic, whereby glucose is known to inhibit cellulase transcription (by microarray analysis), and therefore, mutants with deregulated cellulase expression were able to survive through cellulase hydrolysis of PASC. Isolation of mutant strains required the introduction of random mutations into the bacterial chromosome using a mutagen such as N-methyl-N′-nitro-N-nitroso-guanidine (NTG) to create a diverse mutant pool (>10⁴ CFU/mL), selection for deregulated cellulase expression mutants by plating on agar containing only PASC as a carbon source and DoG, followed by screening of growth colonies for cellulase expression as defined by PASC clearing zones. Hits were sub-cultured and subjected to fluorescence-based cellulase activity assays and clearing zone analysis of PASC overlays following growth on glucose agar plates containing PASC. Final hits were defined by fold-improvement over the parent strain in the fluorescence assay and having positive clearing zones in the PASC overlay assay. The positive hits were further tested for fitness by fermentation profiling with and without excess glucose present in growth medium.

### Example 2

### Bacterial Strains and Media

All bacterial strains used were genus Clostridium, species phytofermentans, strain Q.B. Growth conditions were anaerobic (Oxygen maintained at 0 to less than 1 ppm.), at 35° C. Growth medium QM contained (per liter): 10.6 g K₂HPO₄, 1.92 g KH₂PO₄, 4.6 g (NH₄)₂SO₄, 3g Na₃C₆H_{S}O₇, 6g Bacto yeast extract, 1g Cysteine.HCl, adjusted to pH 7.5 with NaOH. Cellobiose (20% w/v) or glucose (40% w/v) stock solution in ddH₂O, filter sterilized, was added post autoclaving to a required final concentration of 2%. Salts solution (Table 3) was prepared at 100× in ddH2O, and added to QM medium post autoclaving. Screening plates were prepared with PASC (0.5% w/v) in the place of cellobiose/glucose and added pre-autoclaving, 1× salts solution in ddH2O, 75 mM DoG, (added post-autoclaving of medium), and agar (1.5% w/v).

TABLE 3
100x Salts solution

| 100X salt components | Gram per Liter |
|---|---|
| Na₃C₆H₅O₇ | 10 |
| CaC1₂•2H₂O | 0.5 |
| MgSO₄•7H₂O | 6 |
| FeSO₄•7H₂O | 0.4 |
| CoSO₄•H₂O | 0.2 |
| ZnSO₄•7H₂O | 0.2 |
| NiCl₂ | 0.2 |
| MnSO₄•H₂O | 0.5 |
| CuSO₄•5H₂O | 0.04 |
| KAl(SO₄)₂•12H₂O | 0.04 |
| H₃BO₃ | 0.04 |
| (NH₄)₆Mo₇O₂₄•4H₂O | 0.04 |
| Na₂SeO₃ | 0.04 |

Each salt was added in order (allowing each to dissolve prior to next addition) to 1000g ddH₂O and mixed.

Fermentation medium (FM) contained (per liter): 20g Bacto yeast extract, 1.5 g Corn Steep Powder, 1.36 g KH₂PO₄, 2g Na₃C₆H₅O₇, 1.2 g C₆H₈O₇.H2O, 0.5 g (NH₄)₂SO₄, 1g NaCl, 1g Cysteine.HCl, 11.45 g TES (Tris EthaneSulfonic acid), adjusted to pH 8.0 with NaOH.

### Example 3

### Assays and Reagents

Fluorescent cellulase activity assays were performed with 4-methylumbilliferone (4Mu) substrates (glucopyranoside and/or cellobioside) prepared in 50 mM sodium citrate buffer with 5 mM calcium chloride, pH 6.4. Gluconolactone, (25 mM), was supplemented to half the samples as a beta-glucosidase inhibitor to differentiate between cellulase deregulation and beta-glucosidase over-expression mutants. Nine parts substrate, (180 μL 1.1 mM 4Mu-glycoside) were incubated with 1 part cell broth (20 μL overnight cultures, OD₆₆₀ nm measured at the time of assay) at 37° C. in air for 18 h. Fluorescence emission was measured (Ex.360 nm/Em.465 nm) for each timepoint. The raw ARFU (RFU₁₈ₕ minus RFU₀ₕ) was normalized to cell density (measured by OD at 660 nm) and then divided by the value of the parent strain control to obtain a fold improvement over the parent.

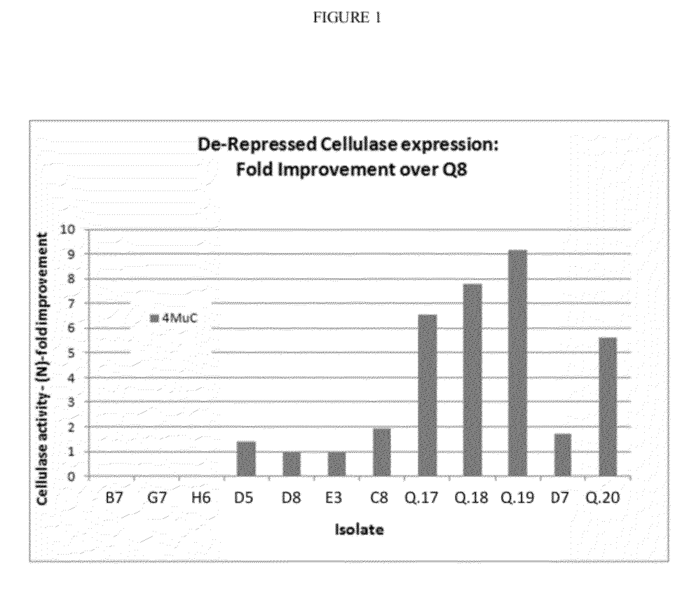

FIG. 1 shows cellulase activity (using 4Mu-Cellobioside as a substrate) produced by mutant strains under cellulase repressing conditions. Kinetic plots of activity per unit time were normalized (divided by) the cell density and plotted. Strains that exhibited two standard deviations higher than the parent are represented below as a “fold” (y axis) increase above the parent strain.

Clearing zones were identified by growing mutants on QM-glucose (0.2% w/v) agar plates, overlaying grown colonies with soft agar 0.5% PASC (0.5% w/v), allowing 2 days for cellulases to clear the PASC (35° C., anaerobic chamber) and staining with Congo red dye (washed with 1M NaCl) to observe clearing zones. Fitness fermentations were conducted in FM containing 5% lignocellulosic feedstock (washed, pretreated solids) and incubated at 35° C. (225 rpm for 7 days) using a 10% inoculum grown in QM. Levels of cellobiose, glucose, arabinose, xylose, acetate, lactate, formate, and ethanol were monitored by HPLC analysis of fermentation samples.

### Example 4

### Mutation Protocol

A single colony (not more than 48h old) of C. phytofermentans Q.8 was resuspended in 5 mL QM medium, and then used to inoculate QM—Cellobiose (2% w/v) at 1:100 (v/v) medium. The culture was grown to mid log phase (OD₆₆₀=0.4 to 0.5) and centrifuged (5000×g for 10 min) to pellet the cells. An NTG solution of 250 mg/mL in 100 mM phosphate buffer (pH 7.4) was prepared and allowed to equilibrate in an anaerobic chamber for at least 16h. The exponential phase cells were resuspended to OD 1.0 in the 100 mM phosphate buffer (pH 7.4). NTG was added to a concentration of 250 μg/ml and the cells incubated in the NTG solution for 1 hr at room temperature. The cells were centrifuged at 5000×g for 10 minutes to pellet. Cell pellets were resuspended to OD 1.0 in 100 mM phosphate buffer and then re-centrifuged. Cell pelleted were resuspended once more to final density OD₆₆₀ 5.0 in 100 mM phosphate buffer/20% glycerol and frozen at −80° C. for storage of the mutant pools. The time and dose of NTG exposure was determined by identifying the conditions which achieve a 99% kill of viable C. phytofermentans cells.

### Example 5

### Screening and Selection

Aliquots were drawn from the NTG treated pools and plated directly on to PASC (0.5% w/v) agar (1.5% w/v) plates containing 75 mM DoG, then incubated at 35° C. for 3-6 days. Resultant colonies were picked into QM-glucose (0.2% w/v) 1 mL broth in 96-deep well plates, foil sealed, then grown overnight at 35° C. in a static incubator. The agar plates were inspected for clearing zones using Congo-Red staining Freezer stock plates were made from the initial overnight cultures by aliquoting into new 96-deep well plates containing QM-glucose (0.2% w/v) 20% w/v glycerol (1 ml). The OD₆₆₀ₙₘ of the remaining culture was measured for each well, and the culture broth used to assay for cellulase activity (4Mu-Glucopyranoside, 4Mu-Cellobioside, 4Mu-cellobioside plus 1 mM gluconolactone). Hits from the fluorescent assay were streaked on QM-glucose (0.2% w/v) agar (1.5% w/v), grown to visible colonies, and assayed once again for clearing zones using PASC (0.5% w/v) overlay and Congo red dye. Hits were further sub-cultured (from freezer stock) for fitness fermentations to assay metabolite profiles and ethanol productivity.

### Example 6

### Fermentation of Biomass by De-Repressed Cellulase Biocatalysts

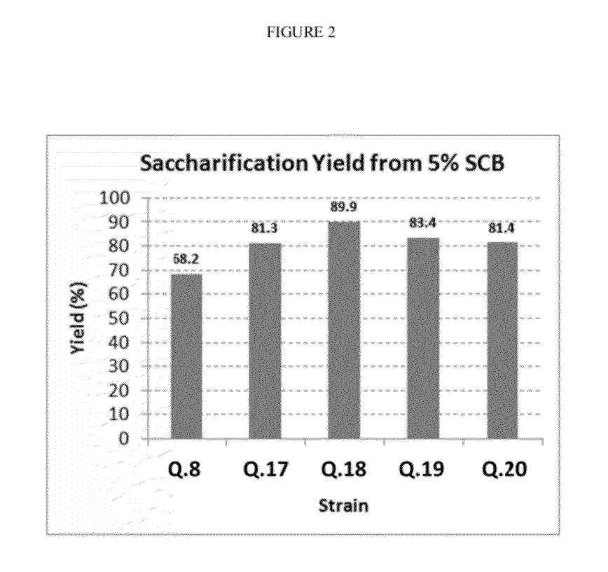

Low enzyme (8.4 FPU/g SCB) assisted fermentations of 5% (w/v) Sugar Cane Bagasse (SCB) were conducted to observe any increase in saccharification of biomass substrate beyond the parent strain Q.B. All enzyme-assisted fermentations were conducted in shake flasks (100 mL culture volume), using FM supplemented with biomass feedstock, incubated anaerobically for 7 days (shaken at 225 rpm) at 35° C. In FIG. 2&3, an increase in saccharification and ethanol titer is observed with all deregulated mutants compared to the parent strain Q.8 (11 g/L, open circles). Strain Q.18 (closed circles) demonstrates the highest titer reaching 14.5 g/L, with Q.17 (open triangle), 0.19 (closed diamond), and Q.20 (closed triangle) reaching about 13 g/L. Much higher saccharification yields are observed with the deregulated cellulase strains (all>80%) due to the constitutive expression of cellulase enzymes, when compared to the parent strain Q.8 (68%) (FIG. 2).

### Example 7

### Site Alterations of Q.17, Q.18, Q.19, and Q.20

Mutants of Clostridium phytofermentans were isolated that express cellulolytic activity under normally repressive conditions for the parent strain (e.g., parent will not express cellulase under identical fermentation conditions); these mutants are termed Q17, Q18, Q19, and Q20. To identify the genetic changes that allowed this phenotype, the genomes of all four strains were sequenced using a commercial “re-sequencing” method from Beckman Coulter. The results are detailed below.

In total, 101 different genes and 26 intergenic regions were found to be mutated in Q17, Q18, Q19, and/or Q20 when compared to the wild type Clostridium phytofermentans strain. Among these 101 genes, 26 were found to contain silent mutations in in Q17, Q18, Q19, and/or Q20, 75 contain missense mutations in Q17, Q18, Q19, and/or Q20, and 4 contain nonsense mutations in Q17, Q18, Q19, and/or Q20. Six genes were identified that contain multiple mutations in at least one of the isolated cellulase mutant strains in comparison to the wild-type strain: Q.17, Q.18, Q.19, and Q.20 were found to contain 2 silent mutations in Cphy_{—}0430, while Q.20 contains an additional missense mutation in Cphy_{—}0430 (Table 8); Q.17, Q.18, Q.19, and Q.20 share two missense mutations in Cphy_{—}1450; Q.17 contains both a silent mutation and a missense mutation in Cphy_{—}1543 (Table 5); Q.17, Q.18, Q.19, and Q.20 each contains two identical missense mutations in Cphy_{—}2868; Q.17, Q.18, Q.19, and Q.20 each contain identical silent and missense mutations in Cphy_{—}1688; and Q.17, Q.18, Q.19, and Q.20 each contain identical silent and missense mutations in Cphy_{—}2885. A missense mutation can be a mutation where the encoded amino acid of a codon is altered by a single nucleotide substitution in the codon. A nonsense mutation can be a single nucleotide substitution that introduces a stop codon in a coding region. Q.17, Q.18, Q.19, and Q.20 contain nonsense mutations in four genes in comparison to Clostridium phytofermentans; however, the same mutations were also found in the parent strain (Q.8). Similarly, of the seventy five genes that were identified with missense mutations in at least one of Q.17, Q.18, Q.19, or Q.20, fifty-five were found to be mutated in all the cellulase mutants (Q.17, Q.18, Q.19, &Q.20) and the parent strain (Q.8). Twenty genes were identified that are mutated in Q.17, Q.18, Q.19, or Q.20 but are wild-type in the parent strain (C. phytofermentans Q.8; Table 4).

Tables 5-8 detail the specific missense mutations that are found in strains Q.17-Q.20 respectively, but were not identified in the parent strain, C. phytofermentans Q.8. SEQ IDs 2-41 contain the wild-type nucleotide or encoded peptide sequences of the identified genes from the wild-type Clostridium phytofermentans strain (as identified in GenBank: CP000885.1). Table 9 details some properties of amino acids that can be used to estimate the severity of any indicated missense mutation.

TABLE 4
Genes that contain missense mutations in the Q.17, Q.18,
Q.19, and/or Q.20 strains but not the Q8 parent strain.

| Gene | Product | Q.17 | Q.18 | Q.19 | Q.20 |
|---|---|---|---|---|---|
| Cphy_2437 | propionyl-CoA carboxylase | ✓ | ✓ | ✓ | ✓ |
| Cphy_3282 | two component AraC family transcriptional regulator | ✓ | ✓ | ✓ | ✓ |
| Cphy_0329 | ROK family glucokinase | ✓ | ✓ | ✓ | ✓ |
| Cphy_3487 | hypothetical protein | ✓ | | | |
| Cphy_1543* | dihydrolipoamide dehydrogenase | ✓ | | | |
| Cphy_2570 | binding-protein-dependent transport systems inner membrane | | ✓ | | |
| | component | | | | |
| Cphy_1682 | ABC transporter related | | ✓ | | |
| Cphy_0056 | hypothetical protein | | ✓ | | |
| Cphy_1910 | TetR family transcriptional regulator | | | ✓ | |
| Cphy_1914 | hypothetical protein (AraC-like) | | | ✓ | |
| Cphy_0430** | glycosyl transferase 36 | | | | ✓ |
| Cphy_2337 | diaminopimelate epimerase | | | | ✓ |
| Cphy_1048 | oxidoreductase domain-containing protein | | | | ✓ |
| Cphy_2965 | hypothetical protein | | | | ✓ |
| Cphy_0987 | desulfoferrodoxin ferrous iron-binding region | | | | ✓ |
| Cphy_1063 | hypothetical protein | | | | ✓ |
| Cphy_0928 | AraC family transcriptional regulator | | | | ✓ |
| Cphy_0788 | phage tape measure protein | | | | ✓ |
| Cphy_2125 | D-isomer specific 2-hydroxyacid dehydrogenase NAD-binding | | | | ✓ |
| Cphy_0935 | HD superfamily phosphohydrolase-like protein | | | | ✓ |

*The Q.17 strain also contains a silent mutation in Cphy_1540.
**Q.17, Q.18, Q.19, and Q.20 also contain 2 silent mutations in Cphy_0430.

TABLE 5
Missense mutations in Clostridium phytofermentans Q.17

| SEQ ID | Cphy | | Gene | Gene | Ref | Genomic | Mut | Ref | Protein | Mut |
|---|---|---|---|---|---|---|---|---|---|---|
| No.² | No. | Gene Name | Start¹ | End¹ | nt | Position¹ | nt | aa | Position | aa |
| SEQ ID | 2437 | propionyl-CoA | 2990661 | 2992094 | G | 2990764 | A | A | 444 | V |
| NO: 2 | | carboxylase | | | | | | | | |
| (nt) | | | | | | | | | | |
| SEQ ID | | | | | | | | | | |
| NO: 3 | | | | | | | | | | |
| (aa) | | | | | | | | | | |
| SEQ ID | 3282 | Two component | 3993730 | 3995337 | A | 3993820 | T | H | 506 | Q |
| NO: 4 | | AraC family | | | | | | | | |
| (nt) | | transcriptional | | | | | | | | |
| SEQ ID | | regulator | | | | | | | | |
| NO: 5 | | | | | | | | | | |
| (aa) | | | | | | | | | | |
| SEQ ID | 0329 | ROK family | 412380 | 413318 | C | 413134 | T | A | 252 | V |
| NO: 6 | | glucokinase | | | | | | | | |
| (nt) | | | | | | | | | | |
| SEQ ID | | | | | | | | | | |
| NO: 7 | | | | | | | | | | |
| (aa) | | | | | | | | | | |
| SEQ ID | 3487 | Hypothetical | 4300804 | 4301628 | G | 4301324 | A | S | 102 | L |
| NO: 8 | | protein | | | | | | | | |
| (nt) | | | | | | | | | | |
| SEQ ID | | | | | | | | | | |
| NO: 9 | | | | | | | | | | |
| (aa) | | | | | | | | | | |
| SEQ ID | 1543* | Dihydrolipoamide | 1894237 | 1895649 | C | 1894430 | T | S | 65 | F |
| NO: 10 | | dehydrogenase | | | | | | | | |
| (nt) | | | | | | | | | | |
| SEQ ID | | | | | | | | | | |
| NO: 11 | | | | | | | | | | |
| (aa) | | | | | | | | | | |

¹Gene Start, Gene End, and Genomic Position refers to locations in Clostridium phytofermentans ISDg, complete genome (GenBank: CP000885.1) deposited Nov. 19, 2007.
²SEQ ID NOs correspond to the wild-type sequences from GenBank: CP000885.1 (nt - nucleotide sequence and aa - encoded peptide sequence).
*Strain Q.17 also contains a silent mutation in Cphy_1543 (G1894578A).

TABLE 6
Missense mutations in Clostridium phytofermentans Q.18

| SEQ ID | Cphy | | Gene | Gene | Ref | Genomic | Mut | Ref | Protein | Mut |
|---|---|---|---|---|---|---|---|---|---|---|
| No.² | No. | Gene Name | Start¹ | End¹ | nt | Position¹ | nt | aa | Position | aa |
| SEQ ID | 2437 | propionyl-CoA | 2990661 | 2992094 | G | 2990764 | A | A | 444 | V |
| NO: 2 | | carboxylase | | | | | | | | |
| (nt) | | | | | | | | | | |
| SEQ ID | | | | | | | | | | |
| NO: 3 | | | | | | | | | | |
| (aa) | | | | | | | | | | |
| SEQ ID | 3282 | Two component | 3993730 | 3995337 | A | 3993820 | T | H | 506 | Q |
| NO: 4 | | AraC family | | | | | | | | |
| (nt) | | transcriptional | | | | | | | | |
| SEQ ID | | regulator | | | | | | | | |
| NO: 5 | | | | | | | | | | |
| (aa) | | | | | | | | | | |
| SEQ ID | 0329 | ROK family | 412380 | 413318 | G | 412716 | A | A | 113 | T |
| NO: 6 | | glucokinase | | | | | | | | |
| (nt) | | | | | | | | | | |
| SEQ ID | | | | | | | | | | |
| NO: 7 | | | | | | | | | | |
| (aa) | | | | | | | | | | |
| SEQ ID | 2570 | binding-protein- | 3137758 | 3138693 | C | 3138530 | T | S | 55 | N |
| NO: 12 | | dependent | | | | | | | | |
| (nt) | | transport systems | | | | | | | | |
| SEQ ID | | inner membrane | | | | | | | | |
| NO: 13 | | component | | | | | | | | |
| (aa) | | | | | | | | | | |
| SEQ ID | 1682 | ABC transporter | 2063482 | 2065323 | G | 2064290 | T | S | 270 | I |
| NO: 14 | | related | | | | | | | | |
| (nt) | | | | | | | | | | |
| SEQ ID | | | | | | | | | | |
| NO: 15 | | | | | | | | | | |
| (aa) | | | | | | | | | | |
| SEQ ID | 0056 | Hypothetical | 81998 | 82324 | C | 82245 | T | S | 83 | F |
| NO: 16 | | protein | | | | | | | | |
| (nt) | | | | | | | | | | |
| SEQ ID | | | | | | | | | | |
| NO: 17 | | | | | | | | | | |
| (aa) | | | | | | | | | | |

¹Gene Start, Gene End, and Genomic Position refers to locations in Clostridium phytofermentans ISDg, complete genome (GenBank: CP000885.1) deposited Nov. 19, 2007.
²SEQ ID NOs correspond to the wild-type sequences from GenBank: CP000885.1 (nt - nucleotide sequence and aa - encoded peptide sequence).

TABLE 7
Missense mutations in Clostridium phytofermentans Q.19

| SEQ ID | Cphy | | Gene | Gene | Ref | Genomic | Mut | Ref | Protein | Mut |
|---|---|---|---|---|---|---|---|---|---|---|
| No.² | No. | Gene Name | Start¹ | End¹ | nt | Position¹ | nt | aa | Position | aa |
| SEQ ID | 2437 | propionyl-CoA | 2990661 | 2992094 | G | 2990764 | A | A | 444 | V |
| NO: 2 | | carboxylase | | | | | | | | |
| (nt) | | | | | | | | | | |
| SEQ ID | | | | | | | | | | |
| NO: 3 | | | | | | | | | | |
| (aa) | | | | | | | | | | |
| SEQ ID | 3282 | Two component | 3993730 | 3995337 | A | 3993820 | T | H | 506 | Q |
| NO: 4 | | AraC family | | | | | | | | |
| (nt) | | transcriptional | | | | | | | | |
| SEQ ID | | regulator | | | | | | | | |
| NO: 5 | | | | | | | | | | |
| (aa) | | | | | | | | | | |
| SEQ ID | 0329 | ROK family | 412380 | 413318 | G | 412716 | A | A | 113 | T |
| NO: 6 | | glucokinase | | | | | | | | |
| (nt) | | | | | | | | | | |
| SEQ ID | | | | | | | | | | |
| NO: 7 | | | | | | | | | | |
| (aa) | | | | | | | | | | |
| SEQ ID | 1910 | TetR family | 2356046 | 2356669 | G | 2356304 | A | E | 87 | K |
| NO: 18 | | transcriptional | | | | | | | | |
| (nt) | | regulator | | | | | | | | |
| SEQ ID | | | | | | | | | | |
| NO: 19 | | | | | | | | | | |
| (aa) | | | | | | | | | | |
| SEQ ID | 1914 | Hypothetical | 2361132 | 2362028 | G | 2361612 | A | E | 161 | K |
| NO: 20 | | protein (AraC- | | | | | | | | |
| (nt) | | like) | | | | | | | | |
| SEQ ID | | | | | | | | | | |
| NO: 21 | | | | | | | | | | |
| (aa) | | | | | | | | | | |

¹Gene Start, Gene End, and Genomic Position refers to locations in Clostridium phytofermentans ISDg, complete genome (GenBank: CP000885.1) deposited Nov. 19, 2007.
²SEQ ID NOs correspond to the wild-type sequences from GenBank: CP000885.1 (nt - nucleotide sequence and aa - encoded peptide sequence).

TABLE 8
Missense mutations in Clostridium phytofermentans Q.20

| SEQ ID | Cphy | | Gene | Gene | Ref | Genomic | Mut | Ref | Protein | Mut |
|---|---|---|---|---|---|---|---|---|---|---|
| No.² | No. | Gene Name | Start¹ | End¹ | nt | Position¹ | nt | aa | Position | aa |
| SEQ ID | 2437 | propionyl-CoA | 2990661 | 2992094 | G | 2990764 | A | A | 444 | V |
| NO: 2 | | carboxylase | | | | | | | | |
| (nt) | | | | | | | | | | |
| SEQ ID | | | | | | | | | | |
| NO: 3 | | | | | | | | | | |
| (aa) | | | | | | | | | | |
| SEQ ID | 3282 | Two component | 3993730 | 3995337 | A | 3993820 | T | H | 506 | Q |
| NO: 4 | | AraC family | | | | | | | | |
| (nt) | | transcriptional | | | | | | | | |
| SEQ ID | | regulator | | | | | | | | |
| NO: 5 | | | | | | | | | | |
| (aa) | | | | | | | | | | |
| SEQ ID | 0329 | ROK family | 412380 | 413318 | G | 412716 | A | A | 113 | T |
| NO: 6 | | glucokinase | | | | | | | | |
| (nt) | | | | | | | | | | |
| SEQ ID | | | | | | | | | | |
| NO: 7 | | | | | | | | | | |
| (aa) | | | | | | | | | | |
| SEQ ID | 0430* | glycosyl | 547250 | 549745 | G | 548216 | A | E | 323 | K |
| NO: 22 | | transferase 36 | | | | | | | | |
| (nt) | | | | | | | | | | |
| SEQ ID | | | | | | | | | | |
| NO: 23 | | | | | | | | | | |
| (aa) | | | | | | | | | | |
| SEQ ID | 2337 | diaminopimelate | 2881225 | 2882073 | G | 2881295 | A | T | 260 | I |
| NO: 24 | | epimerase | | | | | | | | |
| (nt) | | | | | | | | | | |
| SEQ ID | | | | | | | | | | |
| NO: 25 | | | | | | | | | | |
| (aa) | | | | | | | | | | |
| SEQ ID | 1048 | oxidoreductase | 1319045 | 1320175 | C | 1319700 | C | S | 219 | F |
| NO: 26 | | domain- | | | | | | | | |
| (nt) | | containing protein | | | | | | | | |
| SEQ ID | | | | | | | | | | |
| NO: 27 | | | | | | | | | | |
| (aa) | | | | | | | | | | |
| SEQ ID | 2965 | Hypothetical | 3630197 | 3630799 | C | 3630673 | T | V | 43 | I |
| NO: 28 | | protein | | | | | | | | |
| (nt) | | | | | | | | | | |
| SEQ ID | | | | | | | | | | |
| NO: 29 | | | | | | | | | | |
| (aa) | | | | | | | | | | |
| SEQ ID | 0987 | desulfoferrodoxin | 1252420 | 1253794 | C | 1252556 | T | P | 46 | L |
| NO: 30 | | ferrous iron- | | | | | | | | |
| (nt) | | binding region | | | | | | | | |
| SEQ ID | | | | | | | | | | |
| NO: 31 | | | | | | | | | | |
| (aa) | | | | | | | | | | |
| SEQ ID | 1063 | Hypothetical | 1339609 | 1343400 | C | 1339805 | T | S | 66 | F |
| NO: 32 | | protein | | | | | | | | |
| (nt) | | | | | | | | | | |
| SEQ ID | | | | | | | | | | |
| NO: 33 | | | | | | | | | | |
| (aa) | | | | | | | | | | |
| SEQ ID | 0928 | AraC family | 1182272 | 1184572 | C | 1182351 | T | G | 741 | E |
| NO: 34 | | transcriptional | | | | | | | | |
| (nt) | | regulator | | | | | | | | |
| SEQ ID | | | | | | | | | | |
| NO: 35 | | | | | | | | | | |
| (aa) | | | | | | | | | | |
| SEQ ID | 0788 | Phage tape | 1013019 | 1015619 | G | 1014703 | A | G | 562 | E |
| NO: 36 | | measure protein | | | | | | | | |
| (nt) | | | | | | | | | | |
| SEQ ID | | | | | | | | | | |
| NO: 37 | | | | | | | | | | |
| (aa) | | | | | | | | | | |
| SEQ ID | 2125 | D-isomer specific | 2627980 | 2628885 | G | 2628307 | A | V | 110 | I |
| NO: 38 | | 2-hydroxyacid | | | | | | | | |
| (nt) | | dehydrogenase | | | | | | | | |
| SEQ ID | | NAD-binding | | | | | | | | |
| NO: 39 | | | | | | | | | | |
| (aa) | | | | | | | | | | |
| SEQ ID | 0935 | HD superfamily | 1193390 | 1195306 | C | 1193508 | T | S | 40 | F |
| NO: 40 | | phosphohydrolase- | | | | | | | | |
| (nt) | | like protein | | | | | | | | |
| SEQ ID | | | | | | | | | | |
| NO: 41 | | | | | | | | | | |
| (aa) | | | | | | | | | | |

¹Gene Start, Gene End, and Genomic Position refers to locations in Clostridium phytofermentans ISDg, complete genome (GenBank: CP000885.1) deposited Nov. 19, 2007.
²SEQ ID NOs correspond to the wild-type sequences from GenBank: CP000885.1 (nt - nucleotide sequence and aa - encoded peptide sequence).
*Strains Q.17, Q.18, Q.19 & Q.20 also contain 2 silent mutations (G547933A & G548125A) in Cphy_0430.

Three genes were identified that contained missense mutations in all 4 cellulase mutant strains but not the parent Q.8 strain: Cphy_{—}2437 (propionyl-CoA carboxylase), Cphy 0329 (ROK family glucokinase) and Cphy 3282 (2 component AraC family transcriptional regulator).

Cphy_{—}0329 is a putative ROK family glucokinase. ROK proteins were initially defined as a group of proteins such as repressors, as yet uncharacterized open reading frames, and kinases whose primary structures are highly conserved. Members of this family include the xylose operon repressor, xylR, from Bacillus subtilis, Lactobaccilllus pentosus, and Staphylococcus xylosus; the N-acetylglucosamine repressor, nagC, from Escherichia coli; glucokinase from Streptomyces coelicolor; fructokinase from Pediococcus pentosaeceus, Streptococcus mutans, and Zymomonas mobilis; allokinase and mlc from Escherichia coli; hypothetical proteins yajF and yhcI, from Escherichia coli, and the corresponding Haemophilus influenzae proteins. The repressor proteins (e.g., xylR and nagC) from this family possess an N-terminal region not present in the sugar kinases that contains a helix-turn-helix DNA-binding motif.

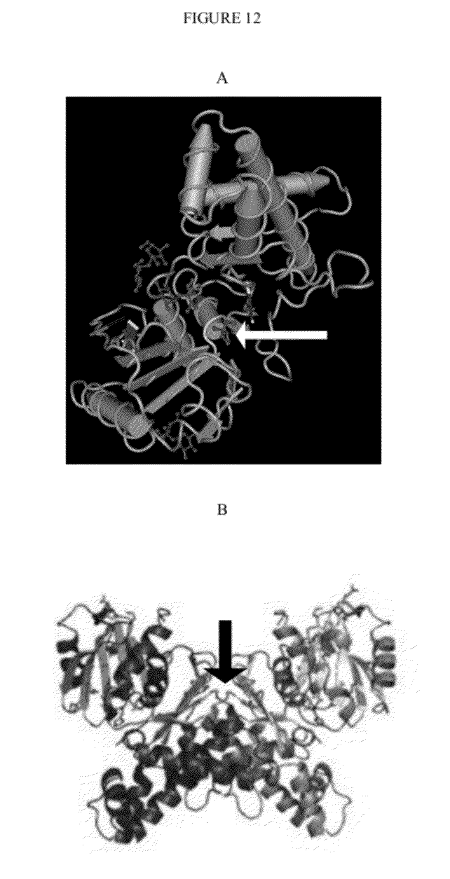

Glucose kinases phosphorylate glucose to produce glucose-6-phosphate (G6P), which is the first step in glycolysis. The levels of G6P are an initial control mechanism for metabolism, known to induce carbon catabolite repression (e.g., shut down cellular biosynthesis, e.g., amino acids and cellulases) when G6P levels rise. Three out of four deregulated cellulase mutants (Q.18, Q.19, and Q.20) have identical missense mutations that change an alanine residue to a threonine residue near the substrate binding site (see Tables 6-8). The mutant residue is located adjacent to a catalytic residue at the sugar binding core (the catalytic residue being aspartic acid) and is predicted to alter the substrate binding pocket. This is based on a protein structure from Streptococcus pneumoniae TIGR4 (PDB:2GUP, in complex with sucrose) which functions as a dimer and adds a phosphate to sucrose (FIG. 12). In FIG. 12A, the location of the mutated residue is pointed to by a white arrow; in FIG. 12B, the mutated residue is indicated by the black arrow. The fourth mutant (found in strain Q.17) has a missense mutation that changes an alanine residue to a valine residue (see Table 5). This mutation is located outside of the conserved region of the gene. Glucose fermentations show that Q.19 and Q.20 can utilize 35% less glucose (26g verses 40g) and 25% less glucose (30g verses 40g) respectively, which is consistent with a decrease in glucose phosphorylation.

Cphy_{—}3282 is a two component AraC family transcriptional regulator. AraC family transcriptional regulators can be activators, repressors, and in some cases, both. Many AraC family transcriptional regulators are self-regulating (e.g., Escherichia coli Ada, Bacillus subtilis AdaA, etc.) and some regulate transcriptions of operons. Cphy_{—}3282 is the first gene of a six gene operon. The next gene in the operon is a histidine kinase; whereas, three of the remaining four genes encode ABC type transport proteins. This is a classical arrangement of a two component regulator controlling the transport of a specific metabolic inducer.

TABLE 9
Amino acid properties

| | 3 - | 1- | Side- | Side- | |
|---|---|---|---|---|---|
| | Letter | Letter | Chain | Chain | Hydropathy |
| Amino Acid | Code | Code | Polarity | Charge | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | Positive (10%) | −3.2 |
| | | | | neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

Table 9 indicates the one and three letter codes for each of the 20 naturally occurring amino acids. Table 9 also indicates some of the physical properties of the amino acids; specifically, polarity, charge at physiological pH, and hydropathy index. The hydropathy index is an indication of the degree to which an amino acid is hydrophobic (positive values) or hydrophilic (negative values). Missense mutations that result in substitution of an amino acid with different properties from the reference amino acid can be more deleterious to the encoded protein functionality; particularly if the mutation is within a conserved region.

### Example 8

### Characterization of Gene Mutations Isolated from Microorganisms with Deregulated Cellulases

Gain of Function Verses Loss of Function

To further characterize the role of individually mutated genes in deregulating cellulase expression, expression and integration constructs are constructed containing the mutated genes according to the methods disclosed throughout. A first experiment is performed in order to test whether an identified mutation is a gain or loss of function mutation. Clostridium phytofermentans Q.8 is transformed with an expression construct containing, for example, a gene mutated according to tables 5-8 and plated onto PASC (phosphoric acid-swollen cellulose) only plates. If the transformed strain grows faster than the parent strain, the gene mutation is a gain of function mutation. If the transformed strain grows at the same rate as, or slower than, the parent strain, the gene mutation is either a loss of function mutation or the mutation alone is insufficient to produce the desired phenotype. A second experiment can be performed to parse this result: replacement of the endogenous gene with the mutant gene through homologous recombination using an integration construct. In this scenario, if the mutated strain grows faster than the parent strain on PASC only plates, the mutation is a loss of function mutation that is sufficient to induce the deregulated cellulases phenotype. Alternatively, if the mutated strain does not grow faster than the parent strain, the observed deregulated cellulases phenotype is most likely caused by either another mutated gene, or a combination of mutated genes. In any of the described experiments, an intermediate result may be observed wherein the mutated or transformed strain grows faster than the parent strain but slower than the isolated mutant strains Q.17, Q.18, Q.19, and/or Q.20. A cohort of mutated genes was identified in each of these strains (see Table 4) and an intermediate result may indicate that two or more gene mutations additively or cooperatively combine to produce the observed phenotype.

Combinatorial Effects of Gene Mutations

The experiments outlined above, performed with each of the mutated genes, generates a list of gene mutations that alone can reduce or eliminate repression of cellulase expression in a microorganism. Combinatorial experiments can also be performed to identify groups of mutated genes that have additive and/or cooperative effects on cellulase expression in the presence of inhibitory compounds such as glucose or DoG. Three genes were identified that were mutated in each of the isolated strains Q.17, Q.18, Q.19, and Q.20. It is possible that a combination of two or three of these mutations is required in order to observe the desired phenotype. If all three mutations are shown to be gain of function mutations, a single expression plasmid encoding all three mutants can be introduced into Clostridium phytofermentans Q.B. Growth rates of the transformed strain on PASC only plates could then be compared to the parent strain and strains expressing only one or two of the genes.

### Example 9

### Expression of Heterologous Genes in C. phytofermentans and Clostridium sp Q.D

Propagation media (QM1) and culture

| | g/L: |
|---|---|
| QM Base Media: | |
| KH₂PO₄ | 1.92 |
| K₂HPO₄ | 10.60 |
| Ammonium sulfate | 4.60 |
| Sodium citrate tribasic * 2H₂O | 3.00 |
| Bacto yeast extract | 6.00 |
| Cysteine | 2.00 |
| 20x Substrate Stock | |
| Maltose | 400.00 |
| 100X QM Salts solution: | |
| MgCl₂•6H₂O | 100 |
| CaCl₂•2H₂O | 15 |
| FeSO₄•7H₂O | 0.125 |

The seed propagation media was prepared according to the protocol above. Base media, salts and substrates were degassed with nitrogen prior to autoclave sterilization. Following sterilization, 94 ml of base media was combined with 1 ml of 100× salts and 5 mls of 20× substrate to achieve final concentrations of 1× for each. All additions were prepared anaerobically and aseptically.

Clostridium phytofermentans or Clostridium sp. Q.D. was propagated in QM media 24 hrs to an active cell density of 2×10⁹ cellsper ml. The cells were concentrated by centrifugation and then transferred into the QM media bottles to achieve an initial cell density of 2×10⁹ cellsper ml for the start of fermentation.

Cultures were then incubated at pH 6.5 and at 35° C. for 120 hr or until fermentations were complete. Product formation was determined by HPLC analysis using refractive index detection. Compositional analysis for the NaOH-treated corn stover was obtained via NREL standard methods using two-stage acid hydrolysis procedures.

Microorganism Modification

Constitutive Expression of pIMPCphy

Plasmids suitable for use in Clostridium phytofermentans were constructed using portions of plasmids obtained from bacterial culture collections (Deutsche Sammlung von Mikroorganismen and Zellkulturen GmbH, Inhoffenstraβe 7 B, 38124 Braunschweig, Germany, hereinafter “DSMZ”). Plasmid pIMP1 is a non-conjugal shuttle vector that can replicate in Escherichia coli and C. phytofermentans; additionally, pIMP1 encodes for resistance to erythromycin (Em^{R}). The origin of transfer for the RK2 conjugal system was obtained from plasmid pRK29O (DSMZ) as DSM 3928, and the other conjugation functions of RK2 were obtained from pRK2013 (DSMZ) as DSM 5599. The polymerase chain reaction (PCR) was used to amplify the 112 base pair origin of transfer region (oriT) from pRK290 using primers that added ClaI restriction sites flanking the oriT region. This DNA fragment was inserted into the ClaI site on pIMP1 to yield plasmid pIMPT. pIMPT was shown to able to be transferred from one strain of E. coli to another when pRK2013 was also present to supply other conjugation functions. PCR was used to amplify the promoter of the alcohol dehydrogenase (Adh) gene Cphy_{—}1029 from the C. phytofermentans chromosome and it was used to replace the promoter of the erythromycin gene in pIMPT to create pIMPTCphy (FIG. 9).

The successful transfer of pIMPTCphy into C. phytofermentans via electroporation was demonstrated by the ability to grow in the presence of 10 μg/mL erythromycin. In addition to phenotypic proof of electroporation provided by the growth on erythromycin, successive plasmid isolations from C. phytofermentans confirmed that the same plasmid was isolated from Clostridium phytofermentans and transferred into E. coli and recovered.

The method of conjugal transfer of pIMPTCphy from E. coli to C. phytofermentans involved constructing an E. coli strain (DHSalpha) that contains both pIMPTCphy and pRK2013. Fresh cells E. coli culture and fresh cells of the C. phytofermentans recipient culture were obtained by growth to mid-log phase using appropriate growth media (L broth and QM1 media respectively). The two bacterial cultures were then centrifuged to yield cell pellets and the pellets resuspended in the same media to obtain cell suspensions that were concentrated about ten-fold having cell densities of about 10¹⁰ cells per ml. These concentrated cell suspensions were then mixed to achieve a donor-to-recipient ratio of five-to-one, after which the cell suspension was spotted onto QM1 agar plates and incubated anaerobically at 30° C. for 24 hours. The cell mixture was removed from the QM1 plate and placed on solid or in liquid QM1 media containing antibiotics that allow the survival of C. phytofermentans recipient cells expressing erythromycin resistance. This was accomplished by using a combination of antibiotics consisting of trimethoprim (20 μg/ml), cycloserine (250 μg/ml), and erythromycin (10 μg/ml). The E. coli donor was unable to survive exposure to these concentrations of trimethoprim and cycloserine, while the C. phytofermentans recipient was unable to survive exposure to this concentration of erythromycin (but could tolerate trimethoprim and cycloserine at these concentrations). Accordingly, after anaerobic incubation on antibiotic-containing plates or liquid media for 5 to 7 days at 30° C., derivatives of C. phytofermentans were obtained that were erythromycin resistant and these C. phytofermentans derivatives were subsequently shown to contain pIMPCphy as demonstrated by PCR analyses.

The vector pIMPCphy was constructed as a shuttle vector for C. phytofermentans and Clostridium. sp. Q.D. It has an Ampicillin-resistance cassette and an Origin of Replication (ori) for selection and replication in E. coli. It contains a Gram-positive origin of replication that can enable the replication of the plasmid in C. phytofermentans. In order to select for the presence of the plasmid, the pIMPCphy carries an erythromycin resistance gene under the control of the C. phytofermentans promoter of the gene Cphyl029. This plasmid can be transferred to C. phytofermentans by electroporation or by transconjugation with an E. coli strain that has a mobilizing plasmid, for example pRK2030. The DNA sequence of pIMPCphy was identified supra as SEQ ID NO: 1. pIMPCphy is an effective replicative vector system for all microbes, including all gram⁺ and gram⁻bacteria, and fungi (including yeasts).

Constitutive Promoter

In a first step, several promoters from C. phytofermentans were chosen that show high expression of their corresponding genes in all growth stages as well as on different substrates. These promoters also work well in Clostridium sp Q.D. A promoter element can be selected by selecting key genes that would necessarily be involved in constitutive pathways (e.g., ribosomal genes, or for ethanol production, alcohol dehydrogenase genes). Examples of promoters from such genes include but are not limited to:

Cphy_{—}1029: iron-containing alcohol dehydrogenase

Cphy_{—}3510: Ig domain-containing protein

Cphy_{—}3925: bifunctional acetaldehyde-CoA/alcohol dehydrogenase

Cloning of Promoter

The different promoters in the upstream regions of the genes were amplified by PCR. The primers for this PCR reaction were chosen in a way that they include the promoter region but do not include the ribosome binding sites of the downstream gene. The primers were engineered to introduce restriction sites at the end of the promoter fragments that are present in the multiple cloning site of pIMPCphy but are otherwise not present in the promoter region itself, for example SalI, BamHI, XmaI, SmaI, EcoRI.

The PCR reaction was performed with a commercially available PCR Kit, e.g. GoTaq® Green Master Mix (Promega Corporation, 2800 Woods Hollow Road, Madison, Wis. 53711 USA), according to the manufacturer's conditions. The reaction is run in a thermal cycler, e.g. Gene Amp System 2400 (PerkinElmer, 940 Winter St., Waltham Mass. 02451 USA). The PCR products were purified with the GenElute™ PCR Clean-Up Kit (Sigma-Aldrich Corp., St. Louis, Mo., USA). Both the purified PCR products as well as the plasmid pIMPCphy were then digested with the corresponding enzymes with the appropriate amounts according to the manufacturer's conditions (restriction enzymes from New England Biolabs, 240 County Road, Ipswich, Mass. 01938 USA and Promega). The PCR products and the plasmid were then analyzed and gel-purified on a Recovery FlashGel (Lonza Biologics, Inc., 101 International Drive, Portsmouth, N.H.03801 USA). The PCR products were subsequently ligated to the plasmid with the Quick Ligation Kit (New England Biolabs) and competent cells of E. coli (DH5a) are transformed with the ligation mixtures and plated on LB plates with 100 μg/ml ampicillin. The plates are incubated overnight at 37° C.

Ampicillin resistant E. coli colonies were picked from the plates and restreaked on new selective plates. After growth at 37° C., liquid LB medium with 100 μg/ml ampicillin was inoculated with a single colony and grown overnight at 37° C. Plasmids were isolated from the liquid culture with the Gene Elute™ Plasmid isolation kit.

Miniprep Kit (Sigma-Aldrich).

Plasmids were checked for the right insert by PCR reaction and restriction digest with the appropriate primers and by restriction enzymes respectively. To ensure the sequence integrity, the insert is sequenced at this step.

Cloning of Genes

One or more genes, which can include each gene's own ribosome binding sites, were amplified via PCR and subsequently digested with the appropriate enzymes as described previously under Cloning of Promoter. Resulting plasmids were also treated with the corresponding restriction enzymes and the amplified genes are mobilized into plasmids through standard ligation. E. coli were transformed with the plasmids and correct inserts were verified from transformants selected on selection plates.

Transconjugation

E. coli DH5α along with the helper plasmid pRK2030, were transformed with the different plasmids discussed above. E. coli colonies with both of the foregoing plasmids were selected on LB plates with 100 μg/ml ampicillin and 50 μg/ml kanamycin after growing overnight at 37° C. Single colonies were obtained after re-streaking on selective plates at 37° C. Growth media for E. coli (e.g. LB or LB supplemented with 1% glucose and 1% cellobiose) was inoculated with a single colony and either grown aerobically at 37° C. or anaerobically at 35° C. overnight. Fresh growth media was inoculated 1:100 with the overnight culture and grown until mid log phase. A C. phytofermentans strain was also grown in the same media until mid log.

The two different cultures, C. phytofermentans and E. coli with pRK2030 and one of the plasmids, were then mixed in different ratios, e.g. 1:1000, 1:100, 1:10, 1:1, 10:1, 100:1, 1000:1. The mating was performed in either liquid media, on plates or on 25 mm Nucleopore Track-Etch Membrane (Whatman, Inc., 800 Centennial Avenue, Piscataway, N.J. 08854 USA) at 35° C. The time was varied between 2h and 24h, and the mating media was the same growth media in which the culture was grown prior to the mating. After the mating procedure, the bacteria mixture was either spread directly onto plates or first grown on liquid media for 6 h to 18h and then plated. The plates contain 10 μg/ml erythromycin as selective agent for C. phytofermentans and 10 μg/ml Trimethoprim, 150 μg/ml Cyclosporin and 100 μg/ml Nalidixic acid as counter selectable media for E. coli.

After 3 to 5 days incubation at 35° C., erythromycin-resistant colonies were picked from the plates and restreaked on fresh selective plates. Single colonies were picked and the presence of the plasmid is confirmed by PCR reaction.

Gene Expression

The expression of the genes on the different plasmids is then tested under conditions where there is little to no expression of the corresponding genes from the chromosomal locus. Positive candidates show constitutive expression of the cloned genes.

Constitutive Expression of a cellulase

pCphyP3510-1163

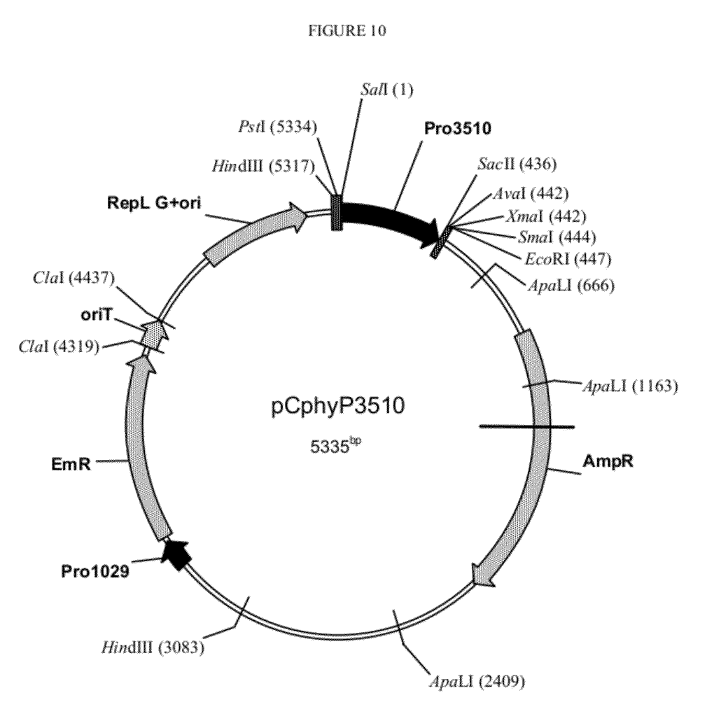

Two primers were chosen to amplify Cphy_{—}1163 using C. phytofermentans genomic DNA as template. The two primers were: cphy_{—}1163F: 5′-CCG CGG AGG AGG GTT TTG TAT GAG TAA AAT CAG AAG AAT AGT TTC-3, (SEQ ID NO. 42) which contained a SacII restriction enzyme site and ribosomal site; and cphy_{—}1163R: CCC GGG TTA GTG GTG GTG GTG GTG GTG TTT TCC ATA ATA TTG CCC TAA TGA (SEQ ID NO. 43), which containing a XmaI site and His-tag. The amplified gene was cloned into Topo-TA first, then digested with SacII and XmaI, the cphy_{—}1163 fragment was gel purified and ligated with pCPHY3510 digested with SacII and XmaI, respectively. The plasmid was transformed into E. coli, purified and then transformed into C. phytofermentans by electroporation. FIGS. 10&11.

Using the methods above genes encoding Cphy_{—}3367, Cphy_{—}3368, Cphy_{—}3202 and Cphy_{—}2058 were cloned into pCphy3510 to produce pCphy3510_{—}3367, pCphy3510_{—}3368, pCphy3510_{—}3202, and pCphy3510_{—}2058 respectively. These vectors were transformed into C. phytofermentans via electroporation as described infra. In addition, genes encoding the heat shock chaperonin proteins, Cphy_{—}3289 and Cphy_{—}3290 were incorporated into pCphy3510. In another embodiment, an endogenous or exogenous gene can be cloned into this vector and used to transform C. phytofermentans, C. sp. Q.D, or another bacteria or fungal cell.

Electroporation Conditions for Clostridium sp. Q.D

No electroporation protocol existed for Clostridium Q.D; therefore a new protocol was established to transfer plasmids into this organism. Based on kill curve experiments, it was noted that cell suspensions containing Clostridium sp. Q.D. will arch at the following condition: 3000V, 600 ohms, and 25 uF. However, the ideal electroporation condition was noted at 2000-2250 V, 600 ohms, and 25 uF; the experimental values for time constants range from 3.2-5.1 ms (average) over the course of 23 independent electroporation procedures. Additionally, the experimental voltage for 2500 V fluctuates from 2400-2500 V based on the freshness of the electroporation buffer.

### Example 10

### Microorganism Modification and Vector Construction

Plasmid Construction

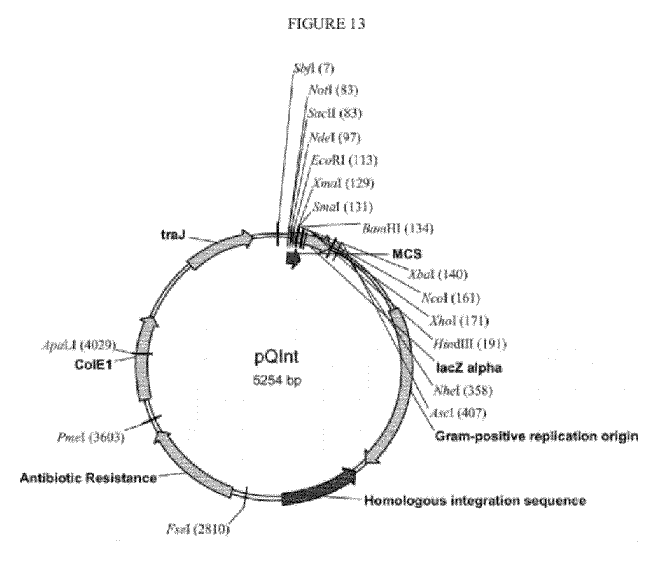

A general illustration of an integrating replicative plasmid, pQInt, is shown in FIG. 13. Identified elements include a Multi-cloning site (MCS) with a LacZ-α reporter for use in E. coli; a gram-positive replication origin; the homologous integration sequence; an antibiotic-resistance cassette; the ColE1 gram-negative replication origin and the traJ origin for conjugal transfer. Several restriction sites are indicated but are not meant to be limiting on any embodiment. The arrangement of the elements can be modified.

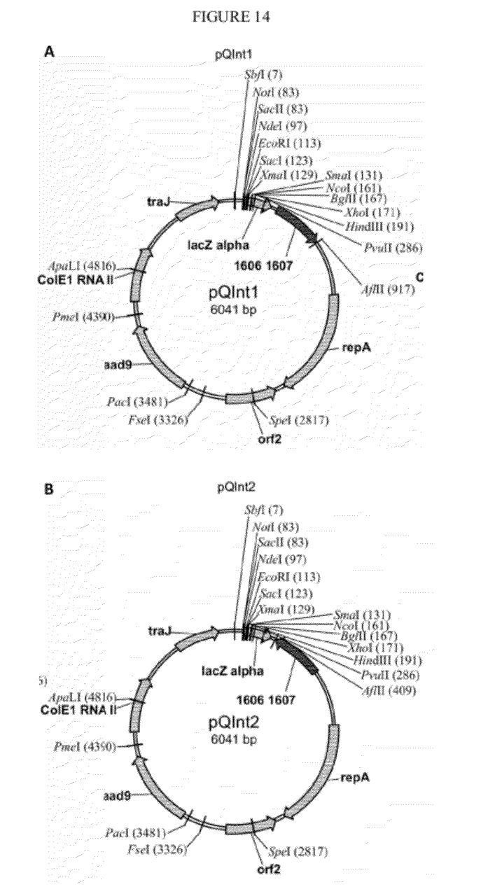

Another embodiment, depicted in FIG. 14A and FIG. 14B, is a map of the plasmids pQInt1 and pQInt2. These plasmids contain gram-negative (Co1E1) and gram-positive (repA/Orf2) replication origins; the bi-functional aad9 spectinomycin-resistance gene; traJ origin for conjugal transfer; LacZ-α/MCS and the 1606-1607 region of chromosomal homology. Since the 1606-1607 region of homology is cloned into a single AscI site, it can be obtained in two different orientations in a single cloning step. Plasmid pQInt2 is identical to pQInt1 except the orientation of the homology region is reversed.

These plasmids consist of five key elements. 1) A gram-negative origin of replication for propagation of the plasmid in E. coli or other gram-negative host(s). 2) A gram-positive replication origin for propagation of the plasmid in gram-positive organisms. In C. phytofermentans, this origin can allow for suitable levels of replication prior to integration. 3) A selectable marker; typically a gene encoding antibiotic resistance. 4) An integration sequence; a sequence of DNA at least 400 base pairs in length and identical to a locus in the host chromosome. This represents a site of integration. 5) A multi-cloning site (“MCS”) with or without a heterologous gene expression cassette cloned. An additional element for conjugal transfer of plasmid DNA is an optional element described in certain embodiments.

Plasmid Utilization

The plasmid is digested with suitable restriction enzyme(s) to allow a heterologous gene expression cassette (“insert”) to be ligated in the MCS. Ligation products are transformed into a suitable cloning host, typically E. coli. Antibiotic resistant transformants are screened to verify the presence of the desired insert. The plasmid is then transformed into C. phytofermentans or other suitable expression host strain. Transformants are selected based on resistance to the appropriate antibiotic. Resistant colonies are propagated in the presence of antibiotic to allow for homologous recombination integration of the plasmid. Integration is verified by a “junction PCR” protocol. This protocol uses either a preparation of host chromosomal DNA or a sample of transformed cells. The junction PCR utilizes one primer that hybridizes to the plasmid backbone flanking the MCS and a second primer that hybridizes to the chromosome flanking the site of integration. The primers can be designed so they are specific; that is, the plasmid primer cannot hybridize to chromosomal sequences and the chromosomal primer cannot hybridize to the plasmid. The ability to amplify a PCR product demonstrates integration at the correct site.

Standard gene expression systems use autonomously replicating plasmids (“episomes” or “episomal plasmids”). Such plasmids are not suitable for use in C. phytofermentans, Clostridium sp. Q.D. and most other Clostridia due to segregational instability. The use of homologous sequences to allow for integration of a replicative gene expression in C. phytofermentans is not usual for transformation.

Use of a series of plasmids each containing a different antibiotic resistance gene, can allow for versatility in cases where certain antibiotics are not suitable for specific organisms. The embodiments use an “integration sequence” which is easily cloned from the chromosome by PCR using primers with tails that encode the appropriate restriction enzyme recognition sequences. This can allow for the targeted integration of the entire plasmid at a chosen locus. The inclusion of a gram-negative replication origin can allow for cloning and the easy propagation of the plasmid in a host such as E. coli. The gram-positive replication origin can allow for a level of replication of the plasmid in C. phytofermentans after transformation and prior to integration. This contrasts with true suicide integration which utilizes non-replicating plasmids. In true suicide integration, the only way to obtain an antibiotic resistant transformant is to have the plasmid integrate immediately after transformation. This is a low probability event. Replication from the gram-positive origin after transformation results in a greater number of transformed cells which makes the integration event statistically more likely.

The integrated plasmid is stable. The transformed strain can be propagated without loss of plasmid DNA. The transformant is evaluated for heterologous gene expression under any suitable conditions. Stability of the integrated DNA is ensured by continuous culture in the presence of the appropriate antibiotic. It is also possible to remove the antibiotic if so desired.

The isolated strains disclosed herein have been deposited in the Agricultural Research Service culture Collection (NRRL), an International Depositary Authority, National Center for Agricultural Utilization Research, Agricultural Research Service, U.S. Department of Agriculture, 1815 North University Street, Peoria, Ill. 61604 U.S.A. on Mar. 9, 2010 (Clostridium phytofermentans Q.8) and Nov. 20, 2010 (Clostridium phytofermentans Q.17, Clostridium phytofermentans Q.18, Clostridium phytofermentans Q.19, and Clostridium phytofermentans Q.20) in accordance with and under the provisions of the Budapest Treaty for the Deposit of Microorganisms; e.g., they will be stored with all the care necessary to keep them viable and uncontaminated for a period of at least five years after the most recent request for the furnishing of a sample of the deposits, and in any case, for a period of at least 30 (thirty) years after the date of deposit or for the enforceable life of any patent which may issue disclosing the cultures plus five years after the last request for a sample from the deposit. The strains were tested by the NRRL and determined to be viable. The NRRL has assigned the following NRRL deposit accession numbers to strains: Clostridium phytofermentans Q.8 (Number NRRL B-50351), Clostridium phytofermentans Q.17 (Designation: Q.8_{—}12_C10; Number NRRL B-50447), Clostridium phytofermentans Q.18 (Designation: Q.8_{—}12_C11; Number NRRL B-50448), Clostridium phytofermentans Q.19 (Designation: Q.8_{—}12_C12; Number NRRL B-50449), and Clostridium phytofermentans Q.20 (Designation: Q.8_{—}12_H9; Number NRRL B-50450). The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them. The deposits are available as required by foreign patent laws in countries wherein counterparts of the subject application, or its progeny, are filed. However, it should be understood that the availability of a deposit does not constitute a license to practice the subject matter disclosed herein in derogation of patent rights granted by governmental action.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure herein. It should be understood that various alternatives to the embodiments of the disclosure described herein can be employed in practicing the described subject matter. It is intended that the following claims define the scope of the disclosure and that methods and structures within the scope of these claims and their equivalents be covered thereby.

What is claimed is:

1. An isolated microorganism that produces a fermentation end-product from a biomass, said microorganism comprising a genetic modification that enables said microorganism to synthesize more cellulases in the presence of an inhibitor molecule than a microorganism of the same species without said genetic modification.

2. The microorganism of claim 1, wherein said inhibitor molecule is glucose or a glucose analog.

3. The microorganism of claim 1, wherein said genetic modification comprises a mutation in one or more genes, wherein at least one of said genes encodes a propionyl-CoA carboxylase, a two component AraC family transcriptional regulator, or a ROK family glucokinase, a homolog of Cphy_{—}3487, a dihydrolipoamide dehydrogenase, a binding-protein-dependent transport systems inner membrane component, an ABC transporter related protein, a homolog of Cphy_{—}0056, a TetR family transcriptional regulator, an AraC-like protein, a glycosyl transferase 36, a diaminopimelate epimerase, an oxidoreductase domain-containing protein, a homolog of Cphy_{—}2965, a desulfoferrodoxin ferrous iron-binding region, a homolog of Cphy_{—}1063, an AraC family transcriptional regulator, a phage tape measure protein, a D-isomer specific 2-hydroxyacid dehydrogenase NAD-binding protein, or an HD superfamily phosphohydrolase-like protein.

4. The microorganism of claim 1, wherein said fermentation end-product is an alcohol.

5. The microorganism of claim 4, wherein said alcohol is ethanol.

6. The microorganism of claim 1, wherein said biomass comprises hemicellulosic or lignocellulosic material.

7. The microorganism of claim 1, wherein said microorganism can hydrolyze and ferment hemicellulosic or lignocellulosic material.

8. The microorganism of claim 1, wherein said microorganism is a Clostridium species.

9. A method of producing a fermentation-end product comprising:
a. providing a biomass in a media;
b. contacting said biomass with an isolated microorganism comprising a genetic modification that enables said microorganism to synthesize more cellulases in the presence of an inhibitor molecule than a microorganism of the same species without said genetic modification; and,
c. allowing sufficient time for said microorganism to produce said fermentation end-product from said biomass.

10. The method of claim 9, wherein said inhibitor molecule is glucose or a glucose analog.

11. The method of claim 9, wherein said fermentation end-product is an alcohol.

12. The method of claim 11, wherein said alcohol is ethanol.

13. The method of claim 9, wherein said biomass comprises hemicellulosic or lignocellulosic material.

14. The method of claim 9, wherein said microorganism can hydrolyze and ferment hemicellulosic or lignocellulosic material.

15. The method of claim 9, wherein said microorganism is a Clostridium species.

16. A plant for producing a fermentation end product comprising:
a. a fermenter, wherein said fermenter is configured to house a biomass in a medium; and,
b. an isolated microorganism comprising a genetic modification that enables said microorganism to synthesize more cellulases in the presence of an inhibitor molecule than a microorganism of the same species without said genetic modification.

17. The plant of claim 16, wherein said inhibitor molecule is glucose or a glucose analog.

18. The plant of claim 16, wherein said fermentation end-product is an alcohol.

19. The plant of claim 18, wherein said alcohol is ethanol.

20. The plant of claim 16, wherein said biomass comprises hemicellulosic or lignocellulosic material and wherein said microorganism can hydrolyze and ferment said hemicellulosic or lignocellulosic material.


SEQUENCE LISTING (43 sequences)

SEQ ID NO: 1 - DNA, 4904 bp, Artificial Sequence
  Description of Artificial Sequence Synthetic plasmid pIMPCphy polynucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaagctttggctaacacacacgccattccaaccaatagttttctcggcataaagccatgctctgacgcttaaatgcactaatgccttaaaaaaacattaaagtctaacacactagacttatttacttcgtaattaagtcgttaaaccgtgtgctctacgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctccttatattttatttcgatttatttgttatttatttaacatttttctattgacctcatcttttctatgtgttattcttttgttaattgtttacaaataatctacgatacatagaaggaggaaaaactagtatactagtatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgatccggccagcctcgcagagcaggattcccgttgagcaccgccaggtgcgaataagggacagtgaagaaggaacacccgctcgcgggtgggcctacttcacctatcctgcccggatcgattatgtcttttgcgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagcttagcttggctgcaggtcgacggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgccgagcgcagcgagtcagtgagcgaggaagcggaaga

SEQ ID NO: 2 - DNA, 1434 bp, Clostridium phytofermentans
  Cphy_2437; propionyl-CoA carboxylase
atgactagtacatcacaactgtcagcaagggacaggattgtcactttgcttgacgagaacagttttgttgaggttggtgctttggttactagaagaagtactgatttcaacctgcaacaaaaagaagtaccttctgatggtgttatcacaggttatggaattattgacggtaatcctgtctatgtttatagccaggatgtagctgtaatgaatggttccataggtgaaatgcacgcaaagaaaatatcaaatttatatgatcttgcaatgaaggttggtgcacctgttatcggtttgattgattgtgccggcttgagattacaggaagcaacggacgcattagctggatttggagatctttatctaaaacaatccttagcatccggggtaatcccacagatcacagcaatttttggtaattgcggaggaggcagtgccatatccgcatctctatgcgactttgtcttcatggaagagaagaacgcaaaattattcgtaaatgctccaaatgcaattctaggaaacaattctactaaatgtgataccgcaacagcttcttttatggctgaagcaggtgttgttgattttgtcgaagcagatgaagaatctgtactaaacagcattcgtaacttagttgcaatgttacctgcaaacaatgaagacgatgcttcttatgaggaatgcaccgatgacttaaatcgtgctttatcctcctttacctctgaacttagtgatacaactcttgcacttaaagatttatctgaccatggtattttcatcgaattgaagaaggcatatgcaaaggacatggtgacaggttttatcaaattaaatggtttaactgttggtgctgtagcaaatcgtactgcgttactcgatgaagatggtaaagtaatcgaaaaatatgatgattctttatctacagcaggttgttataaagcagagaagtttgtaaagttctgcaatgcttttcaaattccggtacttactctaacgaatgttgctggatacagtgcaacaatgaaggatgcaggatccattgctatagcgacagcgaaattaacctatgccttcgcgaatgcaacagtgccaaaagtaaatgtcattttagaaaaggcatacggtagtgcatacataacaatgaattcgaaacatattggtgctgatatggtattcgctttggatggttccatgattggtactatggatgctgaacttgcagcgcagattatgtatgctgataacaaagaagaacaagcgttaaaggctacagagtataaagtattacagcaaagtgcagagtcagcggcaaaacgtggttatgtggatgctattatatcaccagagagtgtaagacaacatgtaatctatgcatttgaaatgttattcacaaagagagagagccgaccaagcaaaaagcatggaacggtttag

SEQ ID NO: 3 - protein, 477 aa, Clostridium phytofermentans
  Cphy_2437; propionyl-CoA carboxylase
MTSTSQLSARDRIVTLLDENSFVEVGALVTRRSTDFNLQQKEVPSDGVITGYGIIDGNPVYVYSQDVAVMNGSIGEMHAKKISNLYDLAMKVGAPVIGLIDCAGLRLQEATDALAGFGDLYLKQSLASGVIPQITAIFGNCGGGSAISASLCDFVFMEEKNAKLFVNAPNAILGNNSTKCDTATASFMAEAGVVDFVEADEESVLNSIRNLVAMLPANNEDDASYEECTDDLNRALSSFTSELSDTTLALKDLSDHGIFIELKKAYAKDMVTGFIKLNGLTVGAVANRTALLDEDGKVIEKYDDSLSTAGCYKAEKFVKFCNAFQIPVLTLTNVAGYSATMKDAGSIAIATAKLTYAFANATVPKVNVILEKAYGSAYITMNSKHIGADMVFALDGSMIGTMDAELAAQIMYADNKEEQALKATEYKVLQQSAESAAKRGYVDAIISPESVRQHVIYAFEMLFTKRESRPSKKHGTV

SEQ ID NO: 4 - DNA, 1608 bp, Clostridium phytofermentans
  Cphy_3282; Two component AraC family transcriptional regulator
atgctaaaagtaatcatagcagatgatgaagataaaatatgccaactaatattcaaattgattgattgggattcccttgatatgaaggttgaggctattgcacataacggtattgatgctttggagttagcaaagttaaataatccggatataataattacagatatcaggatgcccggttatgacggtttggatttcatatcaagaaccagggagataaatccggacatccaatttgttatcatcagcggctatcaacaatttgaatatgcccacaaagcaattaagtatggagtgatcgactatctacttaagccaattaaaaaaaatgaacttttagctactttaaccaagataaagaatcaatatctggaacgggtagatctgttgacgaaagaagaacagtcagagttaagtataagaaataatatttataaattgagaaccgggctgtttaactcggtgctatttaataataggacaaaaaacataaccattgattttataaatagtaattatggatacagatttaaggaaggtttattccaaatcataattgtaaaattagatggcgtgggacggtcttattccactacaattacctttcttgaagagaagctactaaaaattattcataacaatctaaaggaatattgttatgatatggagaattattttgaaaataatataatctattgtttcatgaactacaattttgataacaagaagaatatccggcaccaatgtaagagattaatggatgaattacttttacaaagagaaatatttgaaaatttagaggtaacaattggacttggcacagtggaaactgagattcctatgattggtaattcatacaaggcagcagtctgggcatatcagcaaagattagtacttggtaccaataagataatagagggtaaaataatcacctcaaataactttgttgacagcgatctgtttcatgattttaacaaagatatgaaagcagctctagaaaggctggataaagaatcagttacctcggcaatccattacataagagaaggtttaaggaatcgaccggaaaccagtggacatgaacttcttcaaatgaccaaagagatttgcaatctatttttatttaccatgcgttacaataaatttcccgtagatgggggagataatttccttgagaattttagcatgaatgctaatgacattggttctgcttataaattatatcaatatcttacagacgttattgttaatagcatggataagataatagaagataagagacagagtgatacaagacccatcagggatgcaaagcaatttattcaaacgaactatacgagacagataacacttgacgaagttagcggaaaggttggttttaatgctacatattttagttccttatttaagaaagagactggttacacatttttggaatatctttctgaagtgcgtataaataaggcaaaggaacttttaaaggataccaattatagtgttatggcaatctgtgagcatgtggggtacagtgatattaaacattttacgaaaacgtttgtaaaacacacgaatttgaaaccaaatgagtatcgaaagttatattcatga

SEQ ID NO: 5 - protein, 535 aa, Clostridium phytofermentans
  Cphy_3282; Two component AraC family transcriptional regulator
MLKVIIADDEDKICQLIFKLIDWDSLDMKVEAIAHNGIDALELAKLNNPDIIITDIRMPGYDGLDFISRTREINPDIQFVIISGYQQFEYAHKAIKYGVIDYLLKPIKKNELLATLTKIKNQYLERVDLLTKEEQSELSIRNNIYKLRTGLFNSVLFNNRTKNITIDFINSNYGYRFKEGLFQIIIVKLDGVGRSYSTTITFLEEKLLKIIHNNLKEYCYDMENYFENNIIYCFMNYNFDNKKNIRHQCKRLMDELLLQREIFENLEVTIGLGTVETEIPMIGNSYKAAVWAYQQRLVLGTNKIIEGKIITSNNFVDSDLFHDFNKDMKAALERLDKESVTSAIHYIREGLRNRPETSGHELLQMTKEICNLFLFTMRYNKFPVDGGDNFLENFSMNANDIGSAYKLYQYLTDVIVNSMDKIIEDKRQSDTRPIRDAKQFIQTNYTRQITLDEVSGKVGFNATYFSSLFKKETGYTFLEYLSEVRINKAKELLKDTNYSVMAICEHVGYSDIKHFTKTFVKHTNLKPNEYRKLYS

SEQ ID NO: 6 - DNA, 939 bp, Clostridium phytofermentans
  Cphy_0329; ROK family glucokinase
atggacaagttttgctttggaattgacattggtggcacaacaattaaatgcggattatttacagcgaatggtgaattaaaagaaaaatgggagattccttctagaacagaaaatggcggaattcaagtaccacaggatgtagcggatacgattgatgccaaattaaaagaattatccattgagaaaaaggatgttcttggagttggtattggtgtacccggtccaattaccgaagatggaactgtcttaaaatgtgctaatctaggttgggatatttttaatgtaaatgaaaaaatgagtgcacttaccggactaaaggtagcaactgcaaatgatgccaatgttgctgctctaggagaaatgtggatgggcggtggcaaaggttataagaacatcgttatggttactcttggaaccggcgtaggtggcggagttattttaaatggtaagattgttgcaggaagtaatggcggtggtggtgaaatcggccatatgacaatgaatcttgatgagaaagagacatgcggttgcggtaaacatggccatttagagcaatatgcttctgctacaggcattgtacgtcttgctaaaaaacgtttattagatacaagtgttactacttcacttcgtgagcttgccgaggtaacagcgaaagatatttttgatcacgcgaaagcgggagatacggtcgcactagagcttgttgaagaactaggtagatatcttgggttggcattatctcatgttgctgctgcggttgatccacaggtatttgtaattggtggtggtgtatcaagagctggctctatgttacttgatgtgatttcaaaatattataatcagaatatcatattcgcactatcaaataaagagttccgtcttgctgaacttggaaatgatgcagggatctatggttgtgcaaaattagtgattggctaa

SEQ ID NO: 7 - protein, 312 aa, Clostridium phytofermentans
  Cphy_0329; ROK family glucokinase
MDKFCFGIDIGGTTIKCGLFTANGELKEKWEIPSRTENGGIQVPQDVADTIDAKLKELSIEKKDVLGVGIGVPGPITEDGTVLKCANLGWDIFNVNEKMSALTGLKVATANDANVAALGEMWMGGGKGYKNIVMVTLGTGVGGGVILNGKIVAGSNGGGGEIGHMTMNLDEKETCGCGKHGHLEQYASATGIVRLAKKRLLDTSVTTSLRELAEVTAKDIFDHAKAGDTVALELVEELGRYLGLALSHVAAAVDPQVFVIGGGVSRAGSMLLDVISKYYNQNIIFALSNKEFRLAELGNDAGIYGCAKLVIG

SEQ ID NO: 8 - DNA, 825 bp, Clostridium phytofermentans
  Cphy_3487; Hypothetical polynucleotide
atgagcgtggatgtaatggaaaagtcattaaacgaaaaaatcatcagcgtaaaccaaattcgagagattttggagatgtacaacatagggaaaaaaccattggcgaagttattggggtggggagagaccacagtaattcgctatctggaaggagatattccaacttccgaatatacagaaaagttacaagagattgcagaaagcccaatgtactattatcgtattcttatggagaaccaaagtaaaattaccaatgttgctttcaagaaaagcaaaagagcagtattggaggctatgatgcgttcgaagcttcatgtagcaacacaatatattattaatcaggcaaatgctgaaataaatgcaaggcagattcaatatatattgttctactctcaaatactttctctagtttttttgggagaagagatatttgaagaagatgcacagctgacttacaatcagatgccttatttaaacctctatgaggctctaaagaagcaagggattcgtacaattgaaatgaaccctgataagttaagtagaaatgataaagacattatagattgtgtccacaatgcctgtggttggtatgggaataaagcttttttggccatcgcttcagtggagagaaaggctacggaggaattattagaagggcttaaaagcaaaatcttaacgaaagactatttaagaaatgtatacggtaggatgtttggtcaattccaaataaaacgctatcaagattttccaaagtatctacagcagaggcttattcaggcaattggaaaagacgtattttctgtgaatatatataaagagatataa

SEQ ID NO: 9 - protein, 274 aa, Clostridium phytofermentans
  Cphy_3487; Hypothetical protein
MSVDVMEKSLNEKIISVNQIREILEMYNIGKKPLAKLLGWGETTVIRYLEGDIPTSEYTEKLQEIAESPMYYYRILMENQSKITNVAFKKSKRAVLEAMMRSKLHVATQYIINQANAEINARQIQYILFYSQILSLVFLGEEIFEEDAQLTYNQMPYLNLYEALKKQGIRTIEMNPDKLSRNDKDIIDCVHNACGWYGNKAFLAIASVERKATEELLEGLKSKILTKDYLRNVYGRMFGQFQIKRYQDFPKYLQQRLIQAIGKDVFSVNIYKEI

SEQ ID NO: 10 - DNA, 1413 bp, Clostridium phytofermentans
  Cphy_1543; Dihydrolipoamide dehydrogenase
atggagaaaatatatgatttgcttgttattggtgcaggccctggaggatatgtagcagcgattaaggctgcaaagctaggaatgaagacagcagtgatagaaaacagggaagtcggcggtacctgcttaaaccgcggctgcgttcctgcgaaagccatgctgcatgctgcaaaattatatcaggaggttctgtccggagaacagttcggaatcctcgtggaagaggtaagctttgattacggcaaagtgatgtcctataaaaatgagacttctgaaagtctcagacttggagtagaacagctcctaaagggaaataaagtggaacgcttacaagggatcgggacgcttttgaaggatggaagagttagaattaagacaaaggaaggtgaagaaattctccaggcgaaaaatatattgctggcaacaggatcgaagcctgttcttccgcccattgaaggaatccatcttcccggaatcatgaccagcgatgagatgtttcaacttgatcatgtaccggaaagtctacttattataggcggcggggtcataggggtagaatttgccacagtatactcatcatttggttccaaagttacattgttggaggcggaagaaagacttctacctggtctagataaggaaatctcacagaatataaagctgcttttgaaaaagagaggcgttgatattcataccagagcatttgttcagaaaatagaaaaggtggactgtgagtttatctgtacctttttagagaagggaaaggatcaagaaaaagccgaggtccgaaagattccatacttgctttcagctaccggacgtatcccaaatactcatggtctgctggaggagacaacattactggaaatggacagaggtaggattttggtaaatgaaaattttgaaacaagcatgcctaacgtgtttgctatcggtgatgttattggaggaagccaactagcacatgttgcaagttctcagggtatctgtgcagtggaacgaatgaatgggaaagaaccgtcaattgatttatccgtagttccatcctgtgtttatacagatcctgaaattgcatgtgttggaataacagaacaggaagcgaaagagaaaggaatcgagaccgttactggaaagtttttaacacatgccaacagtaaatcattgataacaaaggaagagagaggctttgttaaagttgttatagataaggaaacgaacgtattgctgggagcgcagatgatgtgcgccagagcgacagatatgatcggtgagatgggaactgccatttccaataaacttactgcgatgcagcttttaaaggctatgcgggcacatcctacctataatgagtccatagcggaagcattggaggattgtaaccatggtgcgattcatgcgttaccgcgcaggtaa

SEQ ID NO: 11 - protein, 470 aa, Clostridium phytofermentans
  Cphy_1543; Dihydrolipoamide dehydrogenase
MEKIYDLLVIGAGPGGYVAAIKAAKLGMKTAVIENREVGGTCLNRGCVPAKAMLHAAKLYQEVLSGEQFGILVEEVSFDYGKVMSYKNETSESLRLGVEQLLKGNKVERLQGIGTLLKDGRVRIKTKEGEEILQAKNILLATGSKPVLPPIEGIHLPGIMTSDEMFQLDHVPESLLIIGGGVIGVEFATVYSSFGSKVTLLEAEERLLPGLDKEISQNIKLLLKKRGVDIHTRAFVQKIEKVDCEFICTFLEKGKDQEKAEVRKIPYLLSATGRIPNTHGLLEETTLLEMDRGRILVNENFETSMPNVFAIGDVIGGSQLAHVASSQGICAVERMNGKEPSIDLSVVPSCVYTDPEIACVGITEQEAKEKGIETVTGKFLTHANSKSLITKEERGFVKVVIDKETNVLLGAQMMCARATDMIGEMGTAISNKLTAMQLLKAMRAHPTYNESIAEALEDCNHGAIHALPRR

SEQ ID NO: 12 - DNA, 936 bp, Clostridium phytofermentans
  Cphy_2570; binding-protein-dependent transport systems inner membrane component
atgaaatctgatacggtaaaaattgtttcaaagcgtaagtctagaattaattcaatcaacctgcctactgagattggcattaatattatacttggtctcttttgtttgatttgcgttgttccgtttatatttgttgtcattatttcttttacttcagaagagagtatacgagagattggatactcctttaccccgaccaaatggtcactggaagcatataaattcgcattcaattccagcaaagcaatttggcgtgcctactttaacagcttttttattactattctgggtacgatattaagtgttttgatttgtgtactatattcttatccattattccgaaaggattttaagtatcgaaaattctttaccttcttctgtttctttactatgttgtttgggggaggcttagtaccaacctattatgttagtaagaatatcttgggcttaagcgataattatgcggcgttaattgtaccggcgttatttaatccgtttaatattattgttatgcgtactttctttcaaagttcggtgccgacggatatcattgaagctgcagcaattgatggcagcggagaatataacaccttgttcaaaataatcgttccgattgcaaagcccggaattgctaccattgcattactgaatgcactggcatactggaatgaatggtatctagcaatgctgtatatccgaactgaaagtctttatccactgcagtatttgctgatgagaacgcagaatcagattgactttttaaccaggaatgccgccatgctgggttcggagatttcaaagcttgtacatgatttaccgcagcaaaatttaagaatggcgcttgctgtacttattgtagtaccgattgcctttgcctatccattcttccagcggtttatcatatcgggtcttacaataggatcagtaaaagggtaa

SEQ ID NO: 13 - protein, 311 aa, Clostridium phytofermentans
  Cphy_2570; binding-protein-dependent transport systems inner membrane component
MKSDTVKIVSKRKSRINSINLPTEIGINIILGLFCLICVVPFIFVVIISFTSEESIREIGYSFTPTKWSLEAYKFAFNSSKAIWRAYFNSFFITILGTILSVLICVLYSYPLFRKDFKYRKFFTFFCFFTMLFGGGLVPTYYVSKNILGLSDNYAALIVPALFNPFNIIVMRTFFQSSVPTDIIEAAAIDGSGEYNTLFKIIVPIAKPGIATIALLNALAYWNEWYLAMLYIRTESLYPLQYLLMRTQNQIDFLTRNAAMLGSEISKLVHDLPQQNLRMALAVLIVVPIAFAYPFFQRFIISGLTIGSVKG

SEQ ID NO: 14 - DNA, 1842 bp, Clostridium phytofermentans
  Cphy_1682; ABC transporter related
atgagccataaagaagaatataaattagggcaatctaatcgtagaaaacaaatgggaccaccaggaccaggtatgggcggcgctggtgagaaagccaaagactttaaaacaacatgggtaaagttacttaagtattgtaaaaaatattgggcagttatggtaattgcgttaagttgcgtagtgatgggtactgtattaacgttaattggtcctgacaaattatcagaacttactgatttgattacaaagggaattgttactggaatcgatttagatggtgttgctagaattggttggacattggtaatattttatggacttagttttctgctgtcattaatacaaggtgtggtgatggctacgattacacaaaatgtatcgaaacagttacgtggagatatttctgctaagattaatcgtttgccgatgtggttttataataagacaacaacaggagatgtactttctcgtgtgacgaatgacgtagataccattgggcaatctctaaatcaaagtgtggggacattagtttctgctattactttattagttggatctttgattatgatgttaaagacaaatgtatttatgacaattacagcgattgttgcaacttgtattggatttgtattgatgatgcttatcatgggaaaatcacaaaaatactttgtacgtcagcaacagcatctaggtgaaatcaatggacatattgaagaaatttatgatgggcataccattgtaaaagcttataatggtgaagccaaagcacgagagaaatttgtttatttaaataatgaacttcgagaaagtaattttatgtctcaatgtttatcagggttaatgatgcctctgatgtcgtttattggtaacttcggttatgtcgcagtttgtgtagttggtgccgttcttgtaatgaataacacgatttcctttggagtaatcgtggcttttatattatatgtaagatattttacacaaccattaagtcagcttgcacaagctgcccagtctttgcaatcggctgcagcagcaggagaacgtgtgtttgaattccttgaagcggaagaaatggaagatgagttggcaaagacaagaaagcttgaaaatgtgcaaggtaaggttgattttgaacatgtaaattttggctatgaaggttctgataaagcaatcattaatgatttctcggtttccaccaagccaggtcaaaaagttgcaattgttggcccaactggagcagggaaaactaccattgtaaatcttttaatgcgtttccatgaaatacaaagtggaactattaagattgataatattcctgttcatcagttacgacgtgaagatgttcatgaacaattttgcatggtacttcaagatacgtggatttttgaaggaacggtccgtgaaaatctagtatatagtacagaaaatgtttctgagcaaacgttagaagaggcatgtaaagcagttggcttgcatcattttatacgtacgcttccacatggatatgatacgatgttaaatgatcaattgagtctatcggcaggacaaaaacagcagcttacaatagcgagagccatgattgcagaccgaccaatgttaattcttgatgaagctacaagttctgtagatactcgtactgagttaatcattcaaaatgctatggatgagttaatgaaaggacgtacttcatttattattgctcataggctatcaactattaaaaatgcggatttgattcttgttatgaaagatggtgatatcatagagagcggaaaccataacgaattaatgaaacaaggcggattctatgcagacctatataatagtcaattcgatgcggcgtaa

SEQ ID NO: 15 - protein, 613 aa, Clostridium phytofermentans
  Cphy_1682; ABC transporter related
MSHKEEYKLGQSNRRKQMGPPGPGMGGAGEKAKDFKTTWVKLLKYCKKYWAVMVIALSCVVMGTVLTLIGPDKLSELTDLITKGIVTGIDLDGVARIGWTLVIFYGLSFLLSLIQGVVMATITQNVSKQLRGDISAKINRLPMWFYNKTTTGDVLSRVTNDVDTIGQSLNQSVGTLVSAITLLVGSLIMMLKTNVFMTITAIVATCIGFVLMMLIMGKSQKYFVRQQQHLGEINGHIEEIYDGHTIVKAYNGEAKAREKFVYLNNELRESNFMSQCLSGLMMPLMSFIGNFGYVAVCVVGAVLVMNNTISFGVIVAFILYVRYFTQPLSQLAQAAQSLQSAAAAGERVFEFLEAEEMEDELAKTRKLENVQGKVDFEHVNFGYEGSDKAIINDFSVSTKPGQKVAIVGPTGAGKTTIVNLLMRFHEIQSGTIKIDNIPVHQLRREDVHEQFCMVLQDTWIFEGTVRENLVYSTENVSEQTLEEACKAVGLHHFIRTLPHGYDTMLNDQLSLSAGQKQQLTIARAMIADRPMLILDEATSSVDTRTELIIQNAMDELMKGRTSFIIAHRLSTIKNADLILVMKDGDIIESGNHNELMKQGGFYADLYNSQFDAA

SEQ ID NO: 16 - DNA, 327 bp, Clostridium phytofermentans
  Cphy_0056; Hypothetical polynucleotide
atgataaaaaagcaaattgtttatactataatttttttggtagtactatttacttcaggatgtgaaaaaaaacaagattatttaaatggaactgtttcagaagttttcgatacgtatattatagtaactccaaaagatgatgaattattaaaagagaaagcggaaaaaattgtagtaactaaaactatagctctagcaacgggatttccagatttagatgttggggacgaaattagagtagtatattctggaataaaaaaagaagatgatttaatgattttagatagtgtatttgctgtatataaatctaatgaattaaaatactaa

SEQ ID NO: 17 - protein, 108 aa, Clostridium phytofermentans
  Cphy_0056; Hypothetical protein
MIKKQIVYTIIFLVVLFTSGCEKKQDYLNGTVSEVFDTYIIVTPKDDELLKEKAEKIVVTKTIALATGFPDLDVGDEIRVVYSGIKKEDDLMILDSVFAVYKSNELKY

SEQ ID NO: 18 - DNA, 624 bp, Clostridium phytofermentans
  Cphy_1910; TetR family transcriptional regulator
atgggaagagtaacggaaaataaacaagcaaaacgaaattcaatctttcattcttcgtttgatttgtttcggaccaaaggtttttttcagacatcgatctcggatatcgtgacaaaggctggagttgccaaaggtaccttttatctatattttaaggacaagtatgatttacgagacaagcttattgcatttaaagcaggaactttgtttcatgcagctgaacttaacttaagtgaaacaaatattaaggattttccggaacgactgatatttattgcagatcagattattgatcggttggctatggaacctgattttctaaaatttatatcaaaaaatttaagctggggcgtatttaagactgcattgttaaagggacatgaaaccggagaaacagatttttataatgcgtatttgcatatggtagaggaaagtccttatgaatttaagaatcccgaaattatgttatttatgattgttgaattagtaggttcaagctgttatagttgtattcttgatgatgaaccattaacgatggaagactataagccatatctttacgaggtaattcgaggaatcattcagatgcagatagttaaaaaaaaatctagcaatgaggtttaa

SEQ ID NO: 19 - protein, 207 aa, Clostridium phytofermentans
  Cphy_1910; TetR family transcriptional regulator
MGRVTENKQAKRNSIFHSSFDLFRTKGFFQTSISDIVTKAGVAKGTFYLYFKDKYDLRDKLIAFKAGTLFHAAELNLSETNIKDFPERLIFIADQIIDRLAMEPDFLKFISKNLSWGVFKTALLKGHETGETDFYNAYLHMVEESPYEFKNPEIMLFMIVELVGSSCYSCILDDEPLTMEDYKPYLYEVIRGIIQMQIVKKKSSNEV

SEQ ID NO: 20 - DNA, 897 bp, Clostridium phytofermentans
  Cphy_1914; Hypothetical polynucleotide (AraC-like)
atgcttaatgtcgatagtataataaagaatgattccgtatttgaactctcgagtacgatagattcaatttaccgaacttatcaaagtgagaaagagcaacttttggccgattatgaagtgaaaaagaaagaatgccaacagttattaatagcgattgataatattagaactgcttatttaaccccttacataaatcagctatatcaggaattagaagagtttggaagtcctagtaaaaagcccaattatactgtacttttaacagatcaatcattaaccaaggactggaaagatagtgatattagacgcacctatctgatagctaaaaagcgtttatcaaaatacaaaaatgattcaaaagatatagttacaaaatttctttatgattggacaagcaataagaaaaagttagaggtaggaaatcaagatctaaagaaattacgagaattggtggcattacaaaaacaagaatacttagaagaaataggaaaagtatcggagataataccaaagctcaagctttatcttgatgtattaatcgatttgaaaattaatataaaagagattattttaccagaaacagaagggattcatgcatttttggtagcaaaacacataacagaacagattacgattaggcaatacccagggaccgttaacttagattctattttaaagctagcaaatacatcatatgaaaaacattatttgtttataaaacaagtatttcgtttttattctaatttggtggaactgttggaacactcaatcagtattgacttttgtacaaagaatagcatcacaccgaaggagttttcttggatactacaaaagaaagaaatcatggaagattctgtcaatgaaatgaaaaggaatttattttatatacaaaattag

SEQ ID NO: 21 - protein, 298 aa, Clostridium phytofermentans
  Cphy_1914; Hypothetical protein (AraC-like)
MLNVDSIIKNDSVFELSSTIDSIYRTYQSEKEQLLADYEVKKKECQQLLIAIDNIRTAYLTPYINQLYQELEEFGSPSKKPNYTVLLTDQSLTKDWKDSDIRRTYLIAKKRLSKYKNDSKDIVTKFLYDWTSNKKKLEVGNQDLKKLRELVALQKQEYLEEIGKVSEIIPKLKLYLDVLIDLKINIKEIILPETEGIHAFLVAKHITEQITIRQYPGTVNLDSILKLANTSYEKHYLFIKQVFRFYSNLVELLEHSISIDFCTKNSITPKEFSWILQKKEIMEDSVNEMKRNLFYIQN

SEQ ID NO: 22 - DNA, 2496 bp, Clostridium phytofermentans
  Cphy_0430; glycosyl transferase 36
atgaaatttggattttttgatgatttaaacaaagaatacgttatcacaactcctgaaacgccgtatccttggattaactatcttggatcgcaagcgttcttttccttgatatcaaatactgctggaggttacagtttctataaagatgcaaaactacgtaggattacaagatttcgttataataatgtaccattagatctcggtggtggtcgttattactacttgtacgataatggtgatttttggtctccgggttttgcccctgcaaaaaaggaactagaatattatgagtgtcgtcatggcatgggttatactaaaataacaggaagaagaaatggaattgaaacaaagataaccttctttgtaccattagactataatggtgaagtacataaagtgtcaattgttaatacaagtaatcaagtgaagaatgtgaaattgttttctttcctcgagtggtgtctgtggaatgcacaagaagattctacaaattttcaacgaaatttctcgacaggtgaagtagaagttaaagggtctgtaatctatcataagacagagtataaagaaagacgggatcattatgcatttttctctgtaaatgcaccgattagcggattcgattctgatcgcgagagttttcttggtacctatcatggttttgataacccgcaggtagttgcaaggggagaatcaaataattctgtagcagatggttggtctccaatcgcctctcattgtgtagaaatttccttacagcctaaggaaagcaaggaactagtatttattcttggatatgctgaaaataaattagaagaaaaatgggagtatcaagttggtgaggtagataccgatgggttattactatcttccgtagcgccaaatggtacgaatgttattaataagaagaaagcagaagagatgatagcaaaatatgctacagtaaaggctgttgatcaagcgttactggagcttaaagattattgggataaactgctttcgaaatataatgtaaagtctcacgatgataaactaaatcgtatggtgaatatctggaaccaatatcaatgtatggtaacctttaatttatcaagaagtgcttcctactttgaatctggtattggaaggggaatgggatttcgagattccaatcaggatttacttggatttgttcatcagattccagatcgtgcaagagagagaatcattgatcttgcatccacgcaattaccagacggtggagcatatcatcagtatcaaccattaacgaaaaaaggaaatgatgagattggtggtaatttcaacgatgatccattgtggcttattctagcagttacagcttatataaaggagacaggagactatgatatcttagaagtgaatactccttatgataataagttagaacttgcgaaaccattgtcagatcatttaaaaagagcatttaaccacgtaatagagaatcttggtccacacggactacctttgattggtcgtgcagattggaacgattgtttgaatctaaactgtttttccatggatccaggagaatcttttcaaacagttacaagtaaggatggtaaagtagcggaatctgttatgattgccggaatgtttacttatattggggaagaatatgctattcttatggataaggtaggaaataacgaagagtctattcgcgccatgaaagaagtagagaatatgcgagatgtaattatgaagcatggctatgatggatcttggtttttacgtgcctacgatgattttggaagaaaaattggtagtgatgagtgtgaagaaggaaaaatatttatcgaatcgcaaggcttctgcgttatgggaaaatgcggtcttaacgatgggaaagcacaaaaagctttagattctgtagaaaaacgtttgggtacaaaattcggattagttctaaataatccagcatttacacgatattatgtggaatatggcgagatttctacctatccagctggatataaagaaaatgcaggtatcttctgtcataacaatgcctggattatgtgtgcggaagcagtgataggccgtggggataaagcctttgattactatacaaaaattgcaccagcgtatacagaagaatactctgaaattcatcgtttggagccatatgtctacgctcagatggttgcaggtaaggatgccagacgttttggtgaagcaaaaaattcttggttaaccggaactgcatcttggaattttgtagcgatatcacaatatatacttggaattaaaccagaatatgatggtttaatgattgaccctgcaatcccaactgattgggaggtatatcaaataacacgtgaattccgtggtgataggtatgatattacaatagtaaacccatatcatgtttcgaaaggtgttaagaaactagttgttgatgcgaaagaggtggagggtaatatcattccggtatttggtgacggattagaacataaagtaaaagtcatcttaggttaa

SEQ ID NO: 23 - protein, 831 aa, Clostridium phytofermentans
  Cphy_0430; glycosyl transferase 36
MKFGFFDDLNKEYVITTPETPYPWINYLGSQAFFSLISNTAGGYSFYKDAKLRRITRFRYNNVPLDLGGGRYYYLYDNGDFWSPGFAPAKKELEYYECRHGMGYTKITGRRNGIETKITFFVPLDYNGEVHKVSIVNTSNQVKNVKLFSFLEWCLWNAQEDSTNFQRNFSTGEVEVKGSVIYHKTEYKERRDHYAFFSVNAPISGFDSDRESFLGTYHGFDNPQVVARGESNNSVADGWSPIASHCVEISLQPKESKELVFILGYAENKLEEKWEYQVGEVDTDGLLLSSVAPNGTNVINKKKAEEMIAKYATVKAVDQALLELKDYWDKLLSKYNVKSHDDKLNRMVNIWNQYQCMVTFNLSRSASYFESGIGRGMGFRDSNQDLLGFVHQIPDRARERIIDLASTQLPDGGAYHQYQPLTKKGNDEIGGNFNDDPLWLILAVTAYIKETGDYDILEVNTPYDNKLELAKPLSDHLKRAFNHVIENLGPHGLPLIGRADWNDCLNLNCFSMDPGESFQTVTSKDGKVAESVMIAGMFTYIGEEYAILMDKVGNNEESIRAMKEVENMRDVIMKHGYDGSWFLRAYDDFGRKIGSDECEEGKIFIESQGFCVMGKCGLNDGKAQKALDSVEKRLGTKFGLVLNNPAFTRYYVEYGEISTYPAGYKENAGIFCHNNAWIMCAEAVIGRGDKAFDYYTKIAPAYTEEYSEIHRLEPYVYAQMVAGKDARRFGEAKNSWLTGTASWNFVAISQYILGIKPEYDGLMIDPAIPTDWEVYQITREFRGDRYDITIVNPYHVSKGVKKLVVDAKEVEGNIIPVFGDGLEHKVKVILG

SEQ ID NO: 24 - DNA, 849 bp, Clostridium phytofermentans
  Cphy_2337; diaminopimelate epimerase
atggaatttgtctttgagaaatatcacggaaatggaaacgattatttagtatttgataccaaaaaatttgagtatgagctgcaaggttcacaaattcgtgaaatatgtgatcgtcactttggggtaggttctgacggtattttaatggggccttatgaaaaggaagatggtatttatgtacgtatctttaatccagatggtagtgaagcagagaagagtggaaatggaatcagtatatttgcacagttcttaaaagaccataagtatgtaacagaggatgtaattacgatcaataccttaggagggccaatcgaaattcattacatcaatgataaaaatggtaccttaaatgttgcgatgggtaaagtttcttatatgagtgatgaaattcctgtaacaggtgaatctagagaagtgattgaagaagcaatggaatttcaaggacagaaggtaatcactacctgtttaaccattggaaatccacattgtgtaattataagagacgaattagttaagaaagaagttaagagcttaggcaaaattattgaatctgatgaacacttccctaataaaatcaatgtgcagtttatgcaagttttaaatcgggaagaaatcgtgattgaaatctatgagcgtggagccggatatactttatcttctggtagtagcagttgtgccgcagctaatgttgcctataaattagggttaacagaccgtaaagtgaaagttcatatgcctggtggaactgtagaggtcgaaatcgaggaagatggtatgacttttatgactagtcatgtaaaacgtatcggaaagattattaccgcagatgtttttatagaagaattataa

SEQ ID NO: 25 - protein, 282 aa, Clostridium phytofermentans
  Cphy_2337; diaminopimelate epimerase
MEFVFEKYHGNGNDYLVFDTKKFEYELQGSQIREICDRHFGVGSDGILMGPYEKEDGIYVRIFNPDGSEAEKSGNGISIFAQFLKDHKYVTEDVITINTLGGPIEIHYINDKNGTLNVAMGKVSYMSDEIPVTGESREVIEEAMEFQGQKVITTCLTIGNPHCVIIRDELVKKEVKSLGKIIESDEHFPNKINVQFMQVLNREEIVIEIYERGAGYTLSSGSSSCAAANVAYKLGLTDRKVKVHMPGGTVEVEIEEDGMTFMTSHVKRIGKIITADVFIEEL

SEQ ID NO: 26 - DNA, 1131 bp, Clostridium phytofermentans
  Cphy_1048; oxidoreductase domain-containing polynucleotide
atgaatcagaacactaaagtaagaatagcactgattggcgttggtagtatggggaaaaagtatgctactatgttagacatggataagatatcaaatcttactttaacagccgtttgttgtagaagtaaagaaaatcaaagttgggtaaaggaaaacctaagtgagagcgttaagttatatccaagtagtgaagaattatttgcacatcatgaagaatacgatgcagtactaattgtaactcctcacaaacaacatccaggactagcaataaaagcatttgaattaaagaaacatgtattttgtgataaacccgctggagtttctttactagacgcgcagagaatggaacaggcacaaaaggaatctggatgtaagtatgctatgatgtttcataatcgaacctatcctgtcttaaaaaaggtaaagcagttattggacgatgggtttgttggagaattaaaacgtatacaacttgtgaacaccatttactatcgtacggaatattatcaccaatccggtgattggagaagcagttggcatggggaaggtggaggagcactcatcaaccaaggacaacatattttagattattggcaatggctattcggaatgccatattcaatctatgcttctattccgtttggaaaatacaattcctttgccgtagatgatgaggcgactttactaatggaatacccaaacaaagtgacagccacctttcttctttctacgggtgaaattccaaaagaagaggtattaaccgtagtaggtacgaaaggttgtattaaagtaactggaaatgaaattgagctcacgtgttatagtatggactctatgcaatatggaaaaactgcaaaaactaattcaagagaagagatactgcaaacaaaggaattatttatatgcaatgaaccaaaggagtcttatgagcaaatgcttgtaaactttggggaagcaatccttcgtgggaaagctcttattgcgcccggagaagaaggtacaaaggcattagagttaacaaatgctgcttacctttctgcatgtttgggagagcgtgtaattttaccaatcgatagtactcagtatgaagaactattaaagaaaatgattgaaaatgagaaagttcaatag

SEQ ID NO: 27 - protein, 376 aa, Clostridium phytofermentans
  Cphy_1048; oxidoreductase domain-containing protein
MNQNTKVRIALIGVGSMGKKYATMLDMDKISNLTLTAVCCRSKENQSWVKENLSESVKLYPSSEELFAHHEEYDAVLIVTPHKQHPGLAIKAFELKKHVFCDKPAGVSLLDAQRMEQAQKESGCKYAMMFHNRTYPVLKKVKQLLDDGFVGELKRIQLVNTIYYRTEYYHQSGDWRSSWHGEGGGALINQGQHILDYWQWLFGMPYSIYASIPFGKYNSFAVDDEATLLMEYPNKVTATFLLSTGEIPKEEVLTVVGTKGCIKVTGNEIELTCYSMDSMQYGKTAKTNSREEILQTKELFICNEPKESYEQMLVNFGEAILRGKALIAPGEEGTKALELTNAAYLSACLGERVILPIDSTQYEELLKKMIENEKVQ

SEQ ID NO: 28 - DNA, 603 bp, Clostridium phytofermentans
  Cphy_2965; Hypothetical polynucleotide
atgcaatggttattagacttaatagcaaaacacacaaaagagggagtactagatacagaagctcttacaaaagaactcaatacagagttcccaaagcatgcagtaccaaagacagagtttaatactgtcaatgaacaacttaagactgcaaataatacaattactgatttaaagaagagcaatggtgacaacgagacattgcagacaaccattaagactcacgaaactactatagcaacacttaaggctgaatctgaaaaggttaagaaggagtatgccttaaaggataagctgaaggatttaggggttactgatgctgactatctgatttacaagcatggtggaattgataagtttaactatgataaggacggtaatctgataggccttgaagattccataaagccatacaaagaatctcttccacatatttttaagaatgccaaatcaggaactgattacaatcctgcaggtggaggaagttataccggaaaaaatccatttgcaaaagattctttcaacttgacggagcaagggaaactattaaaagaaaacccggcacaagctaaggagttagcaagtgctgccggaataacaatttaa

SEQ ID NO: 29 - protein, 200 aa, Clostridium phytofermentans
  Cphy_2965; Hypothetical protein
MQWLLDLIAKHTKEGVLDTEALTKELNTEFPKHAVPKTEFNTVNEQLKTANNTITDLKKSNGDNETLQTTIKTHETTIATLKAESEKVKKEYALKDKLKDLGVTDADYLIYKHGGIDKFNYDKDGNLIGLEDSIKPYKESLPHIFKNAKSGTDYNPAGGGSYTGKNPFAKDSFNLTEQGKLLKENPAQAKELASAAGITI

SEQ ID NO: 30 - DNA, 375 bp, Clostridium phytofermentans
  Cphy_0987; desulfoferrodoxin ferrous iron- binding region
atgatgaaattttataaatgcgatgacgacagtctaattattccagttacgaatacttatgcttctgttgaataccttaagaacgcggaagaaataagccctaatactatggaggcaagtacggagaaacacatccctattgtcacctgtagcggtaataccgtaaaagtaaacgttggtagtgttgctcatccaatgacagaagaacattctatcacgaccgtcattttagaaactcggagtggtggacagtataaattcttacaccatggagatgaaccaatcgttcattttgatgtatcctctggtgataaggcaatggctgcttatgcttactgcaatttgcatggtctatggaaagcagatattgattaa

SEQ ID NO: 31 - protein, 124 aa, Clostridium phytofermentans
  Cphy_0987; desulfoferrodoxin ferrous iron- binding region
MMKFYKCDDDSLIIPVTNTYASVEYLKNAEEISPNTMEASTEKHIPIVTCSGNTVKVNVGSVAHPMTEEHSITTVILETRSGGQYKFLHHGDEPIVHFDVSSGDKAMAAYAYCNLHGLWKADID

SEQ ID NO: 32 - DNA, 3792 bp, Clostridium phytofermentans
  Cphy_1063; Hypothetical polynucleotide
atgaaacatcgagttactgcattgcttctttgtctaactatgttattaggaataataggtatccatcctgtaacagtaaaggcaagtgatccatctttccaattaatgataggaacaaaagatggagagaagaaaacaattgattttgtaaatcctgtatttacagcaagtggaggggccatacaaattaagaattctatatcgcttttttctacagggaacaaagcatatgttagtgagggtacgattaagactaatgtcgctgtagttgtaggtagcgacatgaatgttattcaagtcattaatcaatccagtaatggtgcaaaaccatcatttacagaatctactgatgttatgccaccggagggaggatttgttttactagcttacgatgactcctatgcgaatgcagggtttaagtcctttttagcaacaaagttccaagctggtgatagtgtaaagctatacaatggagatgttgaaatatcactagaggaagcactcaacagctaccctgtggaaattccagacggtggaaataataataatcaaacagattttacatataccttagaaaacttagcagttgagactgcattcgaccctaacaaaggaagtactggtgaaacaaaagcaattgattttgtaaatcctgacttttcaggaccagtagcagttaaaaactcaatatccatatttacctatggtaatcgtccatatgtcagcgctggatctatactaaccaatgttgcagttgtagttgataaaaatatgactgtaatccaggtgatcaatcaatcgatcaatggaggaaaaccatcattttcagaatctacagatgttgctgtaccagcgggtggttttattctactagcatgcgatgacagttatgcaaacgcaggatataagagttttcttgctactaagtttaaagctggcgatgctataaaattgaaagtaaatgaaaagacagtaactgtggcggacattttacagttaaccggccaaaatgggaatattaagaagcatgcttccttatcgattgcacaggaaggtatgcaaaccattacagaaagttctttcacaatctcaggtaaagttaataacttagaagatagtgtaacttattccgtaagagtagatcagataaataatgggaaggtatatccaggtgtagttggggcagatggatcttacactgttccagtatcagtagatccaggagcgaattactttgatataacattaattgaaaatggagtagattattctgaaagtacaaagagtgtaatcctattccagagagttagaacatcagagaataaacctattattttatggattgaccagtttgcgagcgttaaaaatttaaatactgtagaaaagattcaaaagatgatggcaaatgcgaaaagagctggaatcactgcattagcattcgacgtaaagggagtcgaaggttatgtctcctataaaaaggcaaccgtaagcaatacaccatatatgacagaaaccaaaaatccaaacaaagcagttgctatggatattgattttcttgaggagatgctagcagaagcacatgcaaatggaattaagttatatgctagttctaatttctttacggaaggtaacattgcaacaaatgattatgcttttgatattagaaatacacatcctgattgggcagaagtatttcagactccagaagataaaggagagttaaaaagcattctaaattcctccagaaactccaccttactttttgtaaatccagcgaatgaagaagttagggcacatgagttggctatagtaaaagatgtacttgaaaactatgctgttgatggtatcatcttagaccgtgctagatatgataatcagtatgcagattttagtaatttgagcaaagagcaatttatggcataccttcagggaaaaggtaaaacattgcagaactggccagacgatgcgttcaagattaaagcagatggctctatggtaaccggacagcattatcttgaatggttatcctatcgtagtactgtgattgaaagctttgtttctgaagtaagaaccctaatagatcaatataaaacatctcaaaatcgaaacatagatttagcagcatatgttggatcatggtatgagtcatattatcaaaatggcgttaactgggcagattcttcttttgaatacaatgaacgccttggtttccctatggaagaactatatgcgaaagagtttgaatattctaagactagttatgttaaacatattgactttattatgacaggatgttattacacaaccgaagcattgatgcaaaaatatacaacattaaataacatcttaataaataaccaagttccattatatgcttctattgatttaacgaatttatcagaagcaccagaccagagaatgatattccaagcagcgtatcagcatagcgaaggatcaatgatctttgatttgtgttttgttgattgggataaaattcgatgcgcgatagcagatattgaatataagaattcagcagtgattggggtttacgatcctaagactaagaatgttttaacagttgataacattgatactgccagagcagaagataaattaacaatttataccgatgcctatggtacaagtacaggtaccaatcaatggggtgtagaagttgtagtagatgcgaaaggtaatgtaatagaattaaaaaaccagaaacaagcggcagactggaactgggcaacaccagaaattaatgatagtacaatacctgtaggtggatttgtattatcaacagttgatcgttctggatctcgtacttatagacagcttctagcaaattcattccatgtcggtgataaagttgcagcagccatcttaactggattcgttgattatgaagaaaaagtatatacttctgcaacagcggatattgaagtgaaggtacaaacctttggccaagatcaaaatactgttgtaaagattggtgataaagaagcaatcgcgaaagaagataataattatttagcgaagcttaacttaagcaatggtgtcaatttgataccaattaccgtttatgtggatggccttaatgttttagagaaaactatatcacttacagctacattaacacctgtaacaccaacgataactcctccagtaaccgggccatctgaaaatggaaatggaaatggtaaagataagactgatgataagaatgttgttatagatgacaaaatgcttgataatatttctataaatgcttctaactttaagctttatgtgggtggtactaaagactatgagcgtcagcttaaggttaatttaccaaactctatcatgaagcttgagaaagaaaaaaaggcaattgttgaaattacttatcaatcttcaaatcctaaggtagctaaagtaggtaacgatggaaatattacagcagtagctgttggtaaagcagtaattactaccaccgtcacagtgaatgataagactactacgttcgaaacagttgtaaacgttttgaaagcttccattaagatagtatacgatgaaagtacaataaaagttggtacaaaagtgactgcacattgtatggcaagtggctatgatatttcaaaaatacagtggggaactacgaagaaaaagattgctgttgttggtaagaataccggcaatcaaaaggtaaatgtctccacacagtccgctggaaaagatgttgttattgtgtatgtcatgaatggaaaagaaaaagtagtattagcagagaaggatattacaatagtgaaataa

SEQ ID NO: 33 - protein, 1263 aa, Clostridium phytofermentans
  Cphy_1063; Hypothetical protein
MKHRVTALLLCLTMLLGIIGIHPVTVKASDPSFQLMIGTKDGEKKTIDFVNPVFTASGGAIQIKNSISLFSTGNKAYVSEGTIKTNVAVVVGSDMNVIQVINQSSNGAKPSFTESTDVMPPEGGFVLLAYDDSYANAGFKSFLATKFQAGDSVKLYNGDVEISLEEALNSYPVEIPDGGNNNNQTDFTYTLENLAVETAFDPNKGSTGETKAIDFVNPDFSGPVAVKNSISIFTYGNRPYVSAGSILTNVAVVVDKNMTVIQVINQSINGGKPSFSESTDVAVPAGGFILLACDDSYANAGYKSFLATKFKAGDAIKLKVNEKTVTVADILQLTGQNGNIKKHASLSIAQEGMQTITESSFTISGKVNNLEDSVTYSVRVDQINNGKVYPGVVGADGSYTVPVSVDPGANYFDITLIENGVDYSESTKSVILFQRVRTSENKPIILWIDQFASVKNLNTVEKIQKMMANAKRAGITALAFDVKGVEGYVSYKKATVSNTPYMTETKNPNKAVAMDIDFLEEMLAEAHANGIKLYASSNFFTEGNIATNDYAFDIRNTHPDWAEVFQTPEDKGELKSILNSSRNSTLLFVNPANEEVRAHELAIVKDVLENYAVDGIILDRARYDNQYADFSNLSKEQFMAYLQGKGKTLQNWPDDAFKIKADGSMVTGQHYLEWLSYRSTVIESFVSEVRTLIDQYKTSQNRNIDLAAYVGSWYESYYQNGVNWADSSFEYNERLGFPMEELYAKEFEYSKTSYVKHIDFIMTGCYYTTEALMQKYTTLNNILINNQVPLYASIDLTNLSEAPDQRMIFQAAYQHSEGSMIFDLCFVDWDKIRCAIADIEYKNSAVIGVYDPKTKNVLTVDNIDTARAEDKLTIYTDAYGTSTGTNQWGVEVVVDAKGNVIELKNQKQAADWNWATPEINDSTIPVGGFVLSTVDRSGSRTYRQLLANSFHVGDKVAAAILTGFVDYEEKVYTSATADIEVKVQTFGQDQNTVVKIGDKEAIAKEDNNYLAKLNLSNGVNLIPITVYVDGLNVLEKTISLTATLTPVTPTITPPVTGPSENGNGNGKDKTDDKNVVIDDKMLDNISINASNFKLYVGGTKDYERQLKVNLPNSIMKLEKEKKAIVEITYQSSNPKVAKVGNDGNITAVAVGKAVITTTVTVNDKTTTFETVVNVLKASIKIVYDESTIKVGTKVTAHCMASGYDISKIQWGTTKKKIAVVGKNTGNQKVNVSTQSAGKDVVIVYVMNGKEKVVLAEKDITIVK

SEQ ID NO: 34 - DNA, 2301 bp, Clostridium phytofermentans
  Cphy_0928; AraC family transcriptional regulator
atgatatctatatccatgaaagaaaaaattcttttatcattcaagaaacataatttttactatcgcattcttcttccattttctatcttcagcattactatagtctgcctaatgtcaggaataaattggtaccgaatagaaaacgaatataataaaaagattattgaatccaaccaacagtttctaaagagggcttctcagctaagtgatcaatatctttatggtaatttcacttccattttaaatagttcttttttggacggttttaagacttcaaaaatggaccgttttattacatatggagatagactaaaaccttctgaatttctaaatttacaccagagtataactaatatctgtgctcaaaactcctcagtaattcaggtttctttatatcagtatgcatcagacctttatctagatagttttgaaggtcttgtttataatgcttccaaaagaccattgaatagtgatcaatcacttaagaattacctatccaccatttccgggcatgagaaagggattctttactacacttctggtcccaattcttttcctgtaaagaaaatagttatgctacgttccgttcccttatataccaattttacaaacggaagtggatttattgcaattactttggatactaatagcctttgggaacaattaaatatgtccactacctccaaagatgattctttttttattttagactctgaaaaaaagcttctcttggaaaaaagcccatcctctatttcctatgaatatctaaaaagtgtgtcggactccaccgaaatttctgcatttttaacttataacggtattcgttaccgtttggatgaaattgtttctgaggattccggatggcattatatctcctgtgtcccaataaatatactcaatgcagaggttagagcacagcatcagcttactcttatgattacactaatttgcatccttctttcactagttatcgttcaaagaatttcaagtaaagcctatcaaccgattgtaaatcttcgaaatcgtctggcaaaaaattattcatctattgaaagcaaagatgatctttctataattgaaggaaccttttcttttttggagaatcaggtagatgatatacagaaaatgctccataaaaatagccaagtcatactctacaaactttttatggatattctcaataaaaaagaactttccgatagccaacttttacacaagttagagcttagtggaattcatattacccaaagtaattattgtctgcttttaatcgaatttgataaacatgtattctacaaactttctcttgaacaacgggaatatttgattacaaagtccgattccctactgaaagatttccttagcgaacctatcattcagaccgcagaggcccagcctgataatcgaattgctattttgctaaatctgaatcctgataattatttatcacttacagaacaactagagcttcttccagaccatttatttgacatttttcatataaaaataaatctggcgttctccgctcctgttctctatctttcagaaatatccaaggtgtattctcgaatttccgaatacatgaaatatttttttcttttcggctatgggaatatatttacagatgaactgatccacaaacttgataatacttcctattctttttcactgcaagattaccagcaaatcgaacgaatggtgagaaatggtactccggatgaattttcatttcttatggaaagttatcaacaaatcatagaatcaggggagtgttcctatcaggaagcgaacaattttttaattcagacttacagaattgcatttaatatagggaaagagttggggttatttgatgatccacataaaaaagatcaaatattaaatgattttaatcatgcaataaactttgcacactctattgagtgtatctgtctggtcgttcagatgtgccatgaatttcttaatgaagaagttcttaatgcggattcttattttatccaacagatacttgagtacattaaaactcatcagaaagaagaaatttccctctctttagttgctcaagtttttcatgtcagtaccggacatttaagccgcttgttcaaaagtgtgaccaaccagaatttttcagcctatgttataaacattaaattagaaactgcagctgaacttctgaataatgaacctgaaaaaagtatttcaaacatagctgctgaacttggatattataccccggcttattttactagattatttaaagaaaagtttggcgttacaccttctcagtttcgtaaaaagtaa

SEQ ID NO: 35 - protein, 766 aa, Clostridium phytofermentans
  Cphy_0928; AraC family transcriptional regulator
MISISMKEKILLSFKKHNFYYRILLPFSIFSITIVCLMSGINWYRIENEYNKKIIESNQQFLKRASQLSDQYLYGNFTSILNSSFLDGFKTSKMDRFITYGDRLKPSEFLNLHQSITNICAQNSSVIQVSLYQYASDLYLDSFEGLVYNASKRPLNSDQSLKNYLSTISGHEKGILYYTSGPNSFPVKKIVMLRSVPLYTNFTNGSGFIAITLDTNSLWEQLNMSTTSKDDSFFILDSEKKLLLEKSPSSISYEYLKSVSDSTEISAFLTYNGIRYRLDEIVSEDSGWHYISCVPINILNAEVRAQHQLTLMITLICILLSLVIVQRISSKAYQPIVNLRNRLAKNYSSIESKDDLSIIEGTFSFLENQVDDIQKMLHKNSQVILYKLFMDILNKKELSDSQLLHKLELSGIHITQSNYCLLLIEFDKHVFYKLSLEQREYLITKSDSLLKDFLSEPIIQTAEAQPDNRIAILLNLNPDNYLSLTEQLELLPDHLFDIFHIKINLAFSAPVLYLSEISKVYSRISEYMKYFFLFGYGNIFTDELIHKLDNTSYSFSLQDYQQIERMVRNGTPDEFSFLMESYQQIIESGECSYQEANNFLIQTYRIAFNIGKELGLFDDPHKKDQILNDFNHAINFAHSIECICLVVQMCHEFLNEEVLNADSYFIQQILEYIKTHQKEEISLSLVAQVFHVSTGHLSRLFKSVTNQNFSAYVINIKLETAAELLNNEPEKSISNIAAELGYYTPAYFTRLFKEKFGVTPSQFRKK

SEQ ID NO: 36 - DNA, 2601 bp, Clostridium phytofermentans
  Cphy_0788; Phage tape measure polynucleotide
atggcagatatatctgcattttttaatttaagtcaaaaaatttccgatacggtcataaatcaaattacaaacgtgtattccaaatcaataagtgcttccgttactcataccgtaaataaggttatagagaactctgtcgtcaatgtcgatagaacaattaataatatagaaaaaatggatcgctctattaatataaatatcggtagattcagaaagtttaaagaagagacgcaagagccaataaaaacggaaagctatggtgaagctattgaaaagataggtatggttgtagaagcaattaataaaatgggtgaagtccttcaaagaattgagacgcaagagtcaataaaaacggaaaaatttgatgaagctattgaaaagatagataaggtcgatgaatcaattaataaaatgggtgaatcccttcaaagaattgagacgcaagagtcaataaaaacggaaaaatttgatgaagctattgaaaagatagataaggtcgatgaatcaattaataaaatggatgaatcccttcaaagaattgagacgcaagagtcaataaaaacggaaaaatttgatgaagctattgaaaagatagataaggtcgatgaatcaattaataaaatgggtgaatcccttcaaaaagctgagatgcaagagccaataaaaacggaaagctttgatgaagctactgaaaagataagtaagaccgaagaagcaattaataaaatagaggaagcccttcaaaaaaccgaggtgaagagcgaggggacaggggcaaaattaaaaaagagttttagttccatatttagttcagtaagagataatttaggaaataattttgctgcggtgggaaaagggataggctctgttggaaatataattaatagtgtgacatcctttggtaccaaatatttagataaggttgaaaatagtaagatattaaagacagcagatgcactagctcaaaccagaacaaaattaacagcaatgacaggtagtcaagcagaggctgatcaatttcaacaaagaatttttgattccgcacaaaattccagaacttcttatgagtcaacagccaatatggttcttgggctaagtgcaaagggctccttttcaaataaggagcagattgttacctttactgaacttgttaataaaaacagtgtattaggaggggcaagcgctgaaggtacgaaaggcgtacaaacagcagttacagaagctatggtttctggaacacttagcggagaaggatttaataatgtattagaaaatgcttatccaattatagaaaacatagcagcataccttaacaaaccaatagaagcagttcaaaaaatgggtgcacaaggtgaaatcagtggtgaattcttagcaaatgctatgtttgcttctgcacaaaaaacgaatgaagagtttagcaaaactcctatgacctttgaacaattgattagttcaataaaagataaagctctgatggtatttcaaccagtattacaaaagataagtgaattgacacaaaatcaagagtttatgaacatgatacaaaatattatgagtgggttaacttttgtgggcgatttggcattaaggattgttggcgtattaataaatgctgcaagtgcaattgttgataattggtcctggattgctcctatgattcttctaattgcagttgcttttggaatatggaagttatctgttctactaagtagttttagtattaaagaattaactgcttccttgctggcatgcccattggtatggattattggtattattatggctattatagcagtcatcaagatcgtaatagatcacataaataaggttggagataagacatacactgtagcaggcgttatttgcggaattttaggtggagtgggagcctttgtttggaacttatttttgggattaggagattttattcttagttttgtaaatctcattgcaaatgcatttataggagttgcgaacttttttgctaatgtatttaagaacccgatatcttccattatctatttatttcaaggaatggctgacggagtattaggtattctggaaggtattgcaaatgcgattgattttgtctttggtagtaattttggtggaacagttgctggttggagaagtggactaaaagacatggctgatgcagcggttcaaaaattagcaccagatgaaaaatatgaacaaaaaattgattatcttaatttatccatggaaagctttggccttacgagagcagaatattcagattggtgggataaggggaatgaatttggtaataaaatcaatgatctctttaaaggaagcacgggagatgacaagagtttcgatgatacttgggatggaattctaaaaaatacagataaaatcgctcataatacggaacttcaaccagatgatttgtcctatttactcgaacttgcagagcgtgatgcaatcaaccgtttcacaacagcggaagttaaaattgatatgggtggtgtttataatacggtatcaagcaaacagaatctggatggaatcgtagagtatctgacggataagttacgagacgaacttaataatactgcaagagcttgtaacgcttaa

SEQ ID NO: 37 - protein, 866 aa, Clostridium phytofermentans
  Cphy_0788; Phage tape measure protein
MADISAFFNLSQKISDTVINQITNVYSKSISASVTHTVNKVIENSVVNVDRTINNIEKMDRSININIGRFRKFKEETQEPIKTESYGEAIEKIGMVVEAINKMGEVLQRIETQESIKTEKFDEAIEKIDKVDESINKMGESLQRIETQESIKTEKFDEAIEKIDKVDESINKMDESLQRIETQESIKTEKFDEAIEKIDKVDESINKMGESLQKAEMQEPIKTESFDEATEKISKTEEAINKIEEALQKTEVKSEGTGAKLKKSFSSIFSSVRDNLGNNFAAVGKGIGSVGNIINSVTSFGTKYLDKVENSKILKTADALAQTRTKLTAMTGSQAEADQFQQRIFDSAQNSRTSYESTANMVLGLSAKGSFSNKEQIVTFTELVNKNSVLGGASAEGTKGVQTAVTEAMVSGTLSGEGFNNVLENAYPIIENIAAYLNKPIEAVQKMGAQGEISGEFLANAMFASAQKTNEEFSKTPMTFEQLISSIKDKALMVFQPVLQKISELTQNQEFMNMIQNIMSGLTFVGDLALRIVGVLINAASAIVDNWSWIAPMILLIAVAFGIWKLSVLLSSFSIKELTASLLACPLVWIIGIIMAIIAVIKIVIDHINKVGDKTYTVAGVICGILGGVGAFVWNLFLGLGDFILSFVNLIANAFIGVANFFANVFKNPISSIIYLFQGMADGVLGILEGIANAIDFVFGSNFGGTVAGWRSGLKDMADAAVQKLAPDEKYEQKIDYLNLSMESFGLTRAEYSDWWDKGNEFGNKINDLFKGSTGDDKSFDDTWDGILKNTDKIAHNTELQPDDLSYLLELAERDAINRFTTAEVKIDMGGVYNTVSSKQNLDGIVEYLTDKLRDELNNTARACNA

SEQ ID NO: 38 - DNA, 906 bp, Clostridium phytofermentans
  Cphy_2125; D-isomer specific 2-hydroxyacid dehydrogenase NAD-binding
atgtttaataaattagttgcaatcgaaccagttagcttaattccatctgcggaacaaaaactttatgattatgcgaaagaagtaatcttatttgatgatatcccaaaggataataatgaaatcattcaccgaatcggtgatgccgatgctgttttacttagctataccagcagtattgataaagaagttctaaatgcctgcccaaatatccgatatatcggtatgtgctgtagcttatattcaccggaaagcgcgaatgtagacattcttaccgccaattctaaaaatatcactgtctatggaatacgtgattatggcgaccagggagttgtggaatatgtaatcagcgaacttgttcggtacttacatgggttcggtgagaaacaatggaaggaacttccgatagagattacagatttaaaagtaggaatcgtaggccttggtacttccgggcaaatgatagccgtagcacttcaggctcttggcgcagatttatattattatagccgcacccgtaaaccggaagaggaggcaagagatataaaatatctaccattgaatgaattacttcagaccgtagatgtagtttgcacctgtctgaataaaaatgtaatcttattccatgaagaacaatttgaatgtcttggtaatcataagatcatgtttaatacttcaataggtccatcccatgacattcctgcacttaccaaatggctttcccatggtgataatgaatttttctgtgataccgctggcgcattaggagatactactggtgagctattatcacatcctcatgtaaattgtatgaaagtatctaccggaaggacaaagcaagcctttgatcgtctgagtgaaaaggtgttgaataatattgagacattcttaaaagaaaataatatgtaa

SEQ ID NO: 39 - protein, 301 aa, Clostridium phytofermentans
  Cphy_2125; D-isomer specific 2-hydroxyacid dehydrogenase NAD-binding
MFNKLVAIEPVSLIPSAEQKLYDYAKEVILFDDIPKDNNEIIHRIGDADAVLLSYTSSIDKEVLNACPNIRYIGMCCSLYSPESANVDILTANSKNITVYGIRDYGDQGVVEYVISELVRYLHGFGEKQWKELPIEITDLKVGIVGLGTSGQMIAVALQALGADLYYYSRTRKPEEEARDIKYLPLNELLQTVDVVCTCLNKNVILFHEEQFECLGNHKIMFNTSIGPSHDIPALTKWLSHGDNEFFCDTAGALGDTTGELLSHPHVNCMKVSTGRTKQAFDRLSEKVLNNIETFLKENNM

SEQ ID NO: 40 - DNA, 1917 bp, Clostridium phytofermentans
  Cphy_0935; HD superfamily phosphohydrolase-like polynucleotide
ttgaaaaacggtatttatttgaacgattctattcatggattaattccattaacagaatatgaaagaagaatcgtttcaacaattggttttaatcgtctccatgacgtatatcaaaattctacggtatatctaacatttccaaccaatcgaaccaaacgatttgaacactccgttggtacgatgaaactttgttctgatatgttttttcaatcgttactgaatacaacagattctatgttgagtgaattctttgaaatctttgatagagaatatgcaacgattttagatagattaagacagcagatggatgtttgtgaagaaaaattaggtagcagactcccgaaggcgatgccgcagattgaactagataagttgagacattcccttataccgaacaatatccctgatcagtataaagtcattcatcttatcctcatacaatctatccgtgcagcagcattgttacatgatattggacatcctccctttagtcatatagtcgaatttgcgttgaaagatgtttacctggaatataaagacaaagcagttagtgaacaggaaaatgcaaaagaatttgtctcgattatgtcgaaatactttgagggtaataagaaattacacgaacaaatgggtgacgagatcagcgagggtattttaagcaaaattatcatgccaatttcagaggatgatgagaaatacgatgaaaatttatttgaacttctgatattagaaagtgtaaaaagaatttttgcagaggatggggcattcaagtatcttcataggattattgattctagtttggacggagaccgcttggattatgtgacgagagatgtaatcaattcgggaaaggattctggaaagatagagtatagcagaattattaatgatatgcagttgtttgtcgagaatggagagatttttttctgtgttccaataaaagcagtaagctcggtagaggattttgttaagcgtagatataatagctataaagatattatttatcatcatagagtcatccaatctgattatattctggaaggaatcgtaaaagatttggtgaagaaatatttgaatgaaacagtatcggaaacacagcgtgacagcgaagtactgataccatttgatatctcaggactttggtttccattgggggataaaaagtcagcaatccaagcaaatgcactttctcaatggaatgactcctggttaacgaccgtactccggcaaatttattatacagaatattatcataatgaagaaatcgaagaaggctctggggattttgtgttggaacaacgtctggcggaattgctgcggaatagcaagcggtatcattccctgattaaacggagtgagaattttaagattattgacgacgctgtgaaactagagataataaaacaaaaaggcaagattgaagagttcctagggaaagaaaatgagccatcttgtggggataaatccacggagctgattcatcaaatgttagagaattctatgaaaaattcttctggctttattttgtcctttatttggagatatagtaaagaaatgaaaatcgaagcctttgaacagatggttagagaaatcgtggaagtcgaaacaaatcatatcgttactaatcttaagacttatgatacggttactttatttaaacggatttcgattggattagattctccaatttatttctataaccataaggaaaagatcagtaccttaaaggatatcagcggaattgctgacattttacagcttgattccgattatctgccagtcttctatatttatattttagcaaaagacaaggatggggttctaaaggagaaaagagaagaactgttaggttgcatcggtaaacgaatcggtgcccagattatgagaagattgggaatttgggaggagatatcatga

SEQ ID NO: 41 - protein, 638 aa, Clostridium phytofermentans
  Cphy_0935; HD superfamily phosphohydrolase- like protein
LKNGIYLNDSIHGLIPLTEYERRIVSTIGFNRLHDVYQNSTVYLTFPTNRTKRFEHSVGTMKLCSDMFFQSLLNTTDSMLSEFFEIFDREYATILDRLRQQMDVCEEKLGSRLPKAMPQIELDKLRHSLIPNNIPDQYKVIHLILIQSIRAAALLHDIGHPPFSHIVEFALKDVYLEYKDKAVSEQENAKEFVSIMSKYFEGNKKLHEQMGDEISEGILSKIIMPISEDDEKYDENLFELLILESVKRIFAEDGAFKYLHRIIDSSLDGDRLDYVTRDVINSGKDSGKIEYSRIINDMQLFVENGEIFFCVPIKAVSSVEDFVKRRYNSYKDIIYHHRVIQSDYILEGIVKDLVKKYLNETVSETQRDSEVLIPFDISGLWFPLGDKKSAIQANALSQWNDSWLTTVLRQIYYTEYYHNEEIEEGSGDFVLEQRLAELLRNSKRYHSLIKRSENFKIIDDAVKLEIIKQKGKIEEFLGKENEPSCGDKSTELIHQMLENSMKNSSGFILSFIWRYSKEMKIEAFEQMVREIVEVETNHIVTNLKTYDTVTLFKRISIGLDSPIYFYNHKEKISTLKDISGIADILQLDSDYLPVFYIYILAKDKDGVLKEKREELLGCIGKRIGAQIMRRLGIWEEIS

SEQ ID NO: 42 - DNA, 45 bp, Artificial Sequence
  Description of Artificial Sequence Synthetic primer
ccgcggaggagggttttgtatgagtaaaatcagaagaatagtttc

SEQ ID NO: 43 - DNA, 51 bp, Artificial Sequence
  Description of Artificial Sequence Synthetic primer
cccgggttagtggtggtggtggtggtgttttccataatattgccctaatga